(19) United States
(12) Patent Application Publication
(10) Pub. No.: US 2026/0176232 A1
(43) Pub. Date: Jun. 25, 2026

(54) PROBENECID COMPOUNDS FOR THE TREATMENT OF INFLAMMASOME-MEDIATED DERMATOLOGICAL CONDITIONS

(71) Applicant: Bacainn Biotherapeutics, Ltd., George Town, Grand Cayman (KY)

(72) Inventors: Chris Murphy, Upton, MA (US); Roland E. Dolle, Eureka, MO (US); Manuel NAVIA, Lexington, MA (US)

(73) Assignee: Bacainn Biotherapeutics, Ltd., George Town, Grand Cayman (KY)

(21) Appl. No.: 19/123,165

(22) PCT Filed: Oct. 31, 2023
(86) PCT No.: PCT/US2023/036494

§ 371 Date: Apr. 22, 2025

Related U.S. Application Data
(60) Provisional application No. 63/422,772, filed on Nov. 4, 2022.

(51) Int. Cl. C07C 311/16; A61K 31/18; A61K 31/426; A61K 31/506; A61K 31/635; A61K 31/64; A61P 17/06; A61P 29/00; A61P 31/20; C07C 311/59; C07D 211/34; C07D 239/42; C07D 277/30

(52) U.S. Cl. CPC C07C 311/16; A61K 31/18; A61K 31/426; A61K 31/506; A61K 31/635; A61K 31/64; A61P 17/06; A61P 29/00; A61P 31/20; C07C 311/59; C07D 211/34; C07D 239/42; C07D 277/30; C07C 2601/14

(57) ABSTRACT

Disclosed herein are methods, compounds, and compositions for treating or preventing inflammasome-mediated dermatological diseases or conditions.

### CROSS-REFERENCE TO RELATED APPLICATIONS

The present claims the benefit of and priority to U.S. Provisional Patent Application No. 63/422,772, filed Nov. 4, 2022.

### TECHNICAL FIELD

The technology of the present disclosure relates generally to methods, compounds, and compositions for treating or preventing inflammasome-mediated dermatological diseases or conditions.

### BACKGROUND

Inflammation is an adaptive response to noxious stimuli. Innate immunity comprises a system of germline-encoded receptors that inspect the intracellular and extracellular compartments for signs of infection and recognize highly conserved microbial motifs or pathogen-associated molecular patterns (PAMPs). These pattern-recognition receptors (PRRs) are expressed by host infection defense cells, such as macrophages, monocytes, dendritic cells, and epithelial cells. Membrane-bound Toll-like receptors (TLRs) and C-type lectins are the PRRs that probe the extracellular milieu and the endosomal compartments for PAMPs, while the cytosol is scanned by intracellular nucleic acid sensors, such as interferon-inducible protein (also known as AIM2) and retinoic acid-inducible gene-like helicases. Activation of these receptors causes proinflammatory cytokine production and type I interferon-dependent antiviral responses via the transcription factor NF-κB.

Nucleotide oligomerization domain (NOD)-like receptors (NLRs) are a type of intracellular PRR that recognize PAMPs and the host-derived signals, DAMPs (danger-associated molecular patterns). NLRs are composed of a conserved central domain, which mediates nucleotide binding and oligomerization, a COOH-terminal leucine-rich domain (LRR), which senses NLR agonists and has an autoinhibitory effect in their absence, and an NH₂-terminal region, which is required for protein-protein interaction. The human NLR gene family is composed of 22 members, which, depending on their NH₂-terminal domains, are classified into four subfamilies: NLRA, NLRB, NLRC, and NLRP. Activation of certain NLRs (NLRP1 (NACHT, LRR and PYD domains-containing protein 1), NLRP3 (NACHT, LRR and PYD domains-containing protein 3), and NLRC4 (NLR family CARD domain-containing protein 4)) leads to assembly of the inflammasome. The inflammasome is an intracellular multimeric protein complex that regulates the maturation and release of proinflammatory cytokines of the IL-1 family (e.g., IL-1β and IL-18) in response to pathogens and endogenous danger signals. Growing evidence indicates that the inflammasome plays a key role in the pathogenesis of dermatological diseases or conditions. Hence, there is a need to develop improved techniques for treating such disorders related to inflammasome activity.

### SUMMARY

In one aspect, the present disclosure provides a compound having the structure of Formula I,

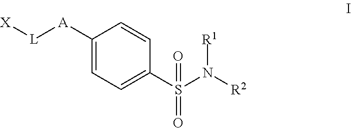

tautomers thereof and/or pharmaceutically acceptable salts thereof;
wherein
A is absent, or is selected from the group consisting of C(O)N(R³), phenylene, oxazolylene, thiazolylene, piperidinylene, and

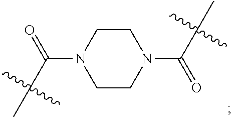 [SMILES: *C(=O)N1CCN(C(*)=O)CC1]

L is absent or C₁₋₁₀ alkylene;
X is H, CHO, COOH, C(O)NR⁴R⁵, COOR⁶, NH₂ or NHR;
R is 2-chloropyrimidin-4-yl;
R¹ and R² are independently a substituted or unsubstituted C₁₋₆ alkyl group, or one of R¹ and R² is H, and the other is cyclohexyl-NH—C(O), or R¹ and R² together are a C₄₋₆ alkylene group and form a 5-, 6-, or 7-member ring with the nitrogen to which they are attached, said ring optionally substituted with a phenyl group;
R³ and R⁴ are independently selected from H or a C₁₋₆ alkyl group;
R⁵ is selected from H, PEG, or a C₁₋₆ alkyl group; and
R⁶ is selected from a substituted or unsubstituted C₁₋₁₀ alkyl, C₂₋₁₀ alkenyl, or C₇₋₁₄ aralkyl group.

In some embodiments, R⁶ is a substituted or unsubstituted C₁₋₁₂ alkyl group. In some embodiments, R⁶ is a C₁₋₆ alkyl group optionally substituted with one or more F, OH, NH₂, NH(C₁₋₄ alkyl), N(C₁₋₄ alkyl)₂ groups. In some embodiments, R⁶ is ethyl, 2-dimethylaminoethyl, or 2,3-dihydoxypropyl.

In some embodiments, A is absent. In some embodiments, A is C(O)N(R³). In some such embodiments, N is attached to L or X. In other embodiments, C is attached to L or X. In some embodiments, R³ is C₁₋₆ alkyl, and in some embodiments, R³ is H or methyl. In some embodiments, A is phenylene, oxazolylene, thiazolylene, piperidinylene or

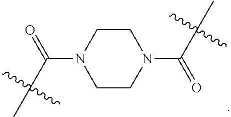 [SMILES: *C(=O)N1CCN(C(*)=O)CC1]

In some embodiments, A is phenylene, oxazolylene, thiazolylene, or piperidinylene.

In some embodiments, L is absent. In some embodiments, L is a C₁₋₁₀ alkylene.

In some embodiments, X is H. In some embodiments, X is CHO. In some embodiments, X is COOH. In some embodiments, X is C(O)NR⁴R⁵. In some embodiments, X is COOR⁶. In some embodiments, X is NH₂ or NHR.

In some embodiments, each of R¹ and R² is independently a C₁₋₆ alkyl, optionally substituted with one or more F, OH, CF₃, C₃₋₇ cycloalkyl group or SO₂-alkyl. In some embodiments, R¹ and R² together are a C₄₋₆ alkylene group and form a 5-, 6-, or 7-member ring with the nitrogen to which they are attached. In some embodiments, R¹ and R² is H, and the other is cyclohexyl-NH—C(O).

In some embodiments, A is absent, or is selected from the group consisting of C(O)N(R³), phenylene, oxazolylene, thiazolylene, and piperidinylene; L is absent or C₁₋₁₀ alkylene; X is H, COOH, or NH₂; R¹ and R² are independently a substituted or unsubstituted C₁₋₆ alkyl group; and R³ is selected from H or a C₁₋₆ alkyl group.

In some embodiments, A is absent and L is C₃₋₁₀ alkylene. In some embodiments, A is phenylene, oxazolylene, thiazolylene, or piperidinylene, and L is absent or a C₁₋₅ alkylene.

In some embodiments, the compound, tautomer thereof, and/or the pharmaceutically acceptable salt thereof is selected from the group consisting of:

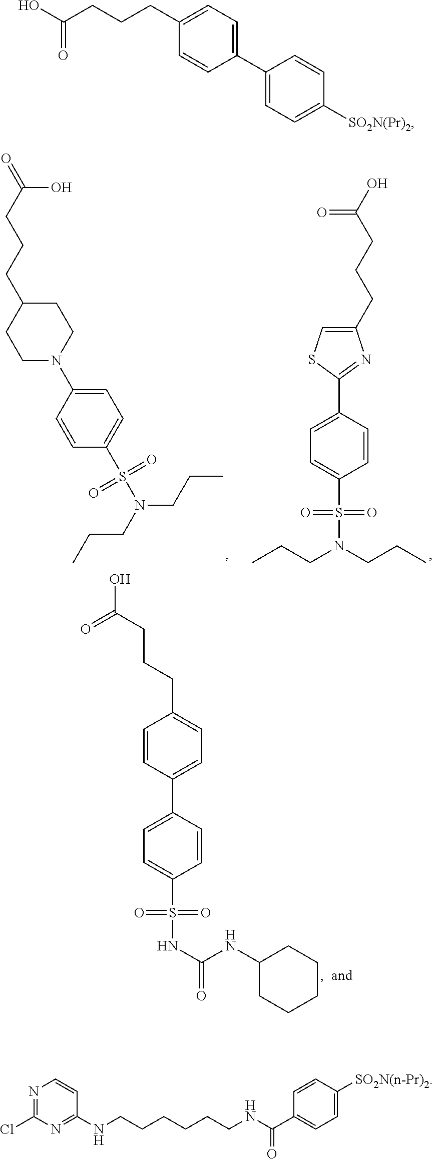

In some embodiments, the present disclosure relates to a pharmaceutical composition comprising the compound, tautomer thereof, and/or pharmaceutically acceptable salt thereof and a pharmaceutically acceptable carrier.

In one aspect, the present disclosure relates to a method for treating or preventing an inflammasome-mediated dermatological disease or condition in a mammalian subject in need thereof, comprising administering to the subject a therapeutically effective amount of a compound, tautomer thereof, and/or pharmaceutically acceptable salt thereof, or a pharmaceutical composition comprising the compound, tautomer thereof, and/or pharmaceutically acceptable salt thereof and a pharmaceutically acceptable carrier.

In some embodiments, the inflammasome-mediated dermatological disease or condition is associated with NLRP1 inflammasome activation and/or NLRP3 inflammasome activation.

In some embodiments, the compound, tauotomer thereof, and/or pharmaceutically acceptable salt or the pharmaceutical composition comprises:

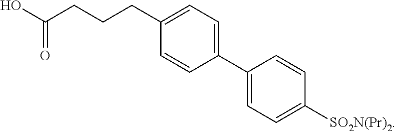

In some embodiments, the compound, tauotomer thereof, and/or pharmaceutically acceptable salt or the pharmaceutical composition comprises:

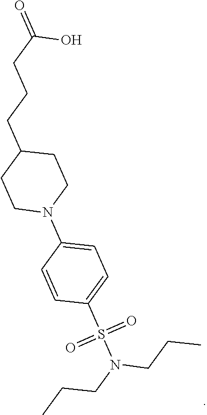 [SMILES: CCCN(CCC)S(=O)(=O)c1ccc(N2CCC(CCCC(=O)O)CC2)cc1]

In some embodiments, the compound, tauotomer thereof, and/or pharmaceutically acceptable salt or the pharmaceutical composition comprises:

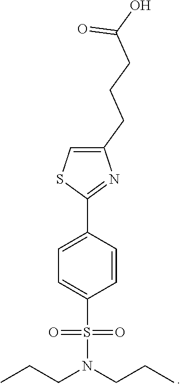 [SMILES: CCCN(CCC)S(=O)(=O)c1ccc(-c2nc(CCCC(=O)O)cs2)cc1]

In some embodiments, the compound, tauotomer thereof, and/or pharmaceutically acceptable salt or the pharmaceutical composition comprises:

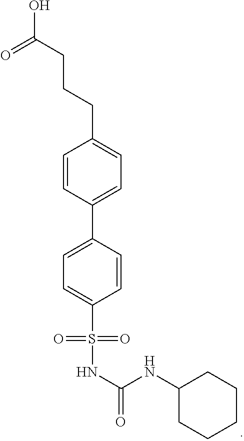 [SMILES: O=C(O)CCCc1ccc(-c2ccc(S(=O)(=O)NC(=O)NC3CCCCC3)cc2)cc1]

In some embodiments, the compound, tauotomer thereof, and/or pharmaceutically acceptable salt or the pharmaceutical composition comprises:

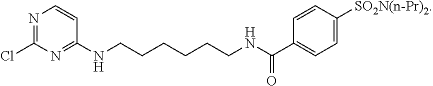

In some embodiments, the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

In some embodiments, the administering step is selected from the group consisting of topical administration, intradermal administration, intranasal administration, intramuscular administration, subcutaneous administration, administration by inhalation, and oral administration.

In some embodiments, the treating or preventing inflammasome-mediated dermatological diseases or conditions comprises reducing the level of one or more inflammatory cytokines in the subject as compared to an untreated control subject.

In some embodiments, the one or more inflammatory cytokines is selected from the group consisting of MCP-1, IL-18, IL-1β, IL-6, TNF-α, and any combination thereof. In some embodiments, the one or more inflammatory cytokines is IL-1β.

In some embodiments, the treating or preventing inflammasome-mediated dermatological disease or condition comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject.

In some embodiments, the treating or preventing inflammasome-mediated dermatological disease or condition comprises reducing apoptosis-associated speck-like protein containing a caspase activating and recruitment domain (ASC) speck formation in the subject as compared to an untreated control subject.

In one aspect, the present disclosure relates to a use of a composition in the preparation of a medicament for treating or preventing an inflammasome-mediated dermatological disease or condition in a subject in need thereof, wherein the composition comprises a therapeutically effective amount of a compound of the present technology, tautomer thereof, and/or pharmaceutically acceptable salt thereof, or a pharmaceutical composition comprising the compound, tautomer thereof, and/or pharmaceutically acceptable salt thereof, and a pharmaceutically acceptable carrier.

In some embodiments, the inflammasome-mediated dermatological disease or condition is associated with NLRP1 inflammasome activation and/or NLRP3 inflammasome activation.

In some embodiments, the compound, tauotomer thereof, and/or pharmaceutically acceptable salt or the pharmaceutical composition comprises:

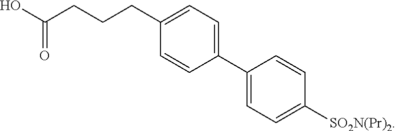

In some embodiments, the compound, tauotomer thereof, and/or pharmaceutically acceptable salt or the pharmaceutical composition comprises:

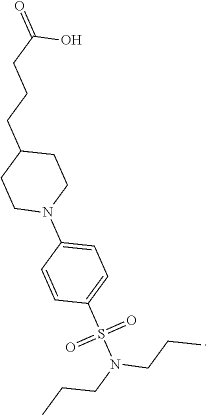 [SMILES: CCCN(CCC)S(=O)(=O)c1ccc(N2CCC(CCCC(=O)O)CC2)cc1]

In some embodiments, the compound, tauotomer thereof, and/or pharmaceutically acceptable salt or the pharmaceutical composition comprises:

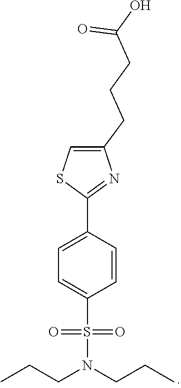 [SMILES: CCCN(CCC)S(=O)(=O)c1ccc(-c2nc(CCCC(=O)O)cs2)cc1]

In some embodiments, the compound, tauotomer thereof, and/or pharmaceutically acceptable salt or the pharmaceutical composition comprises:

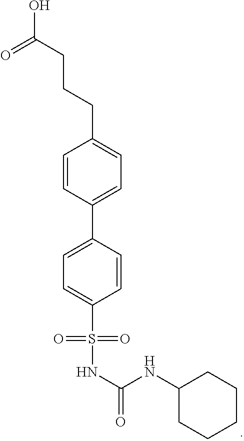 [SMILES: O=C(O)CCCc1ccc(-c2ccc(S(=O)(=O)NC(=O)NC3CCCCC3)cc2)cc1]

In some embodiments, the compound, tauotomer thereof, and/or pharmaceutically acceptable salt or the pharmaceutical composition comprises.

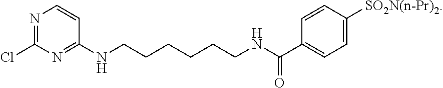

In some embodiments, the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

In some embodiments, the administering step is selected from the group consisting of topical administration, intradermal administration, intranasal administration, intramuscular administration, subcutaneous administration, administration by inhalation, and oral administration.

In some embodiments, the treating or preventing inflammasome-mediated dermatological disease or condition comprises reducing the level of one or more inflammatory cytokines in the subject as compared to an untreated control subject.

In some embodiments, the one or more inflammatory cytokines is selected from the group consisting of MCP-1, IL-18, IL-1β, IL-6, TNF-α, and any combination thereof. In some embodiments, the one or more inflammatory cytokines is IL-1β.

In some embodiments, the treating or preventing dermatological disease or condition comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject.

In some embodiments, the treating or preventing inflammasome-mediated dermatological disease or condition comprises reducing apoptosis-associated speck-like protein containing a caspase activating and recruitment domain (ASC) speck formation in the subject as compared to an untreated control subject.

In one aspect, the present disclosure relates to a compound of the present technology, tautomer thereof, and/or pharmaceutically acceptable salt thereof, or a pharmaceutical composition comprising the compound, tautomer thereof, and/or pharmaceutically acceptable salt thereof, for use in treating or preventing an inflammasome-mediated dermatological disease or condition in a subject in need thereof.

In some embodiments, the inflammasome-mediated dermatological disease or condition is associated with NLRP1 inflammasome activation and/or NLRP3 inflammasome activation.

In some embodiments, the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

In some embodiments, the compound, tauotomer thereof, and/or pharmaceutically acceptable salt or the pharmaceutical composition comprises:

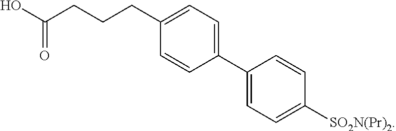

In some embodiments, the compound, tauotomer thereof, and/or pharmaceutically acceptable salt or the pharmaceutical composition comprises:

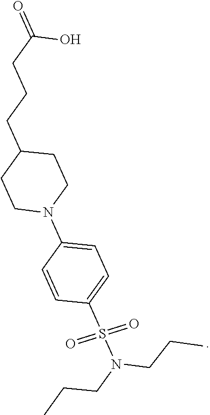 [SMILES: CCCN(CCC)S(=O)(=O)c1ccc(N2CCC(CCCC(=O)O)CC2)cc1]

In some embodiments, the compound, tauotomer thereof, and/or pharmaceutically acceptable salt or the pharmaceutical composition comprises:

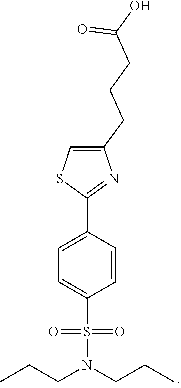 [SMILES: CCCN(CCC)S(=O)(=O)c1ccc(-c2nc(CCCC(=O)O)cs2)cc1]

In some embodiments, the compound, tauotomer thereof, and/or pharmaceutically acceptable salt or the pharmaceutical composition comprises:

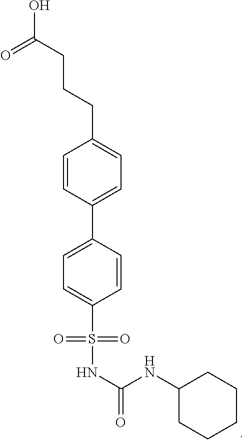 [SMILES: O=C(O)CCCc1ccc(-c2ccc(S(=O)(=O)NC(=O)NC3CCCCC3)cc2)cc1]

In some embodiments, the compound, tauotomer thereof, and/or pharmaceutically acceptable salt or the pharmaceutical composition comprises:

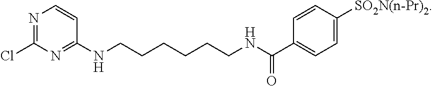

In some embodiments, the inflammasome-mediated dermatological disease or condition is selected from the group consisting of skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

In some embodiments, the administering step is selected from the group consisting of topical administration, intradermal administration, intranasal administration, intramuscular administration, subcutaneous administration, administration by inhalation, and oral administration.

In some embodiments, the treating or preventing inflammasome-mediated dermatological disease or condition comprises reducing the level of one or more inflammatory cytokines in the subject as compared to an untreated control subject.

In some embodiments, the one or more inflammatory cytokines is selected from the group consisting of MCP-1, IL-18, IL-1β, IL-6, TNF-α, and any combination thereof. In some embodiments, the one or more inflammatory cytokines is IL-1β.

In some embodiments, the treating or preventing inflammasome-mediated dermatological disease or condition comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject.

In some embodiments, the treating or preventing of inflammasome-mediated dermatological disease or condition comprises reducing apoptosis-associated speck-like protein containing a caspase activating and recruitment domain (ASC) speck formation in the subject as compared to an untreated control subject.

### BRIEF DESCRIPTION OF THE DRAWINGS

FIG. 1 is a chart showing the dose-response of probenecid analogs on the inhibition of inflammasome activity as assessed by secreted IL-1β ELISA in macrophages pre-activated with lipopolysaccharide (LPS; 100 ng/mL). The macrophages were stimulated with the NLRP3 activator, silica (250 μg/mL). From left to right: the first bar for each compound is non-silica activated; the second bar is silica activated but no compound; the following bars are with the compound at 300 μM, 150 μM, 30 μM, and 3 μM. Prob=probenecid; MCC=MCC950, a specific small molecule inhibitor of NLRP3 inflammasome. From left to right, the compounds listed on the x-axis are as follows: Prob, BT004, BT005, BT006, BT007, BT008, BT009, BT010, BT011, BT026, BT027, BT028, BT029, BT030, BT031, BT032, BT033, BT034, BT035, BT041, BT043, BT052, BT053, BT054, BT055, BT056, BT057, BT058, MCC.

FIG. 2 is a chart showing the dose-response of probenecid analogs BT135, BT136, BT137, BT138, BT139, and BT140 on the inhibition of inflammasome activity as assessed by secreted IL-1β ELISA in macrophages pre-activated with lipopolysaccharide (LPS; 100 ng/mL). The macrophages were stimulated with the NLRP3 activator, nigericin (6 μM). From left to right, the first bar for each compound (cluster) is non-silica activated, the second bar is activated but no compound, the following bars are the compound at 300 μM, 150 μM, 30 μM, and 3 μM. Prob/D=probenecid dissolved in DMSO; Prob/P=probenecid dissolved in PBS. The experiments were performed in triplicate three times.

FIGS. 3A and 3B are charts showing the dose-response of probenecid analogs BT032, BT132, BT133, and BT134, on the inhibition of inflammasome activity as assessed by secreted IL-1β ELISA in macrophages pre-activated with lipopolysaccharide (LPS; 100 ng/mL). The macrophages were stimulated with either the NLRP3 activator, nigericin (6 μM) (FIG. 3A) or silica (250 μg/mL) (FIG. 3B). From left to right, the first bar for each compound (cluster) is non-nigericin or non-silica activated, the second bar is activated but no compound, the following bars are the compound at 300 μM, 150 μM, 30 μM, and 3 μM. The experiments were performed in triplicate.

FIGS. 4A and 4B are charts showing inflammasome activity as determined by measuring secreted IL-1β concentrations in murine immortalized BMDMs pre-activated with lipopolysaccharide (LPS; 100 ng/mL) for 3 h. The macrophages were stimulated with either the NLRP3 activator, nigericin (3 μM) (FIG. 4A) or the NLRP1 agonist L18-MDP (100 μg/mL) (FIG. 4B) and treated or not with BT032 (3.9-350 μM). Secreted IL-1β concentrations were measured by ELISA and are represented as the mean±SEM of the pooled results of 3 independent experiments conducted in triplicate where activity was normalized as percentage of activity as related to the DMSO-treated control cells and non-stimulated (FIG. 4A) or as IL-1β concentration (FIG. 4B) and shown as the curve of the Log [M] BT032 versus normalized responses (variable slope).

FIG. 4C is a chart showing the effects of treatment of macrophages with BT032 on NLRP3 (from left to right: Nigericin, Monosodium Urate (MSU), and Silica)- and NLRP1 (L18-MDP)-induced inflammasome activation, on non-canonical inflammasome activity (LPS (B4)), and on AIM2 (poly dA:dT) and NLRC4 (Flagellin)-mediated inflammasome activation. MCC950 is an NLRP3-specific inhibitor; MSU=monosodium urate; LPS=lipopolysaccharide; BT32=BT032. Secreted IL-1β concentrations were measured by ELISA and are represented as the mean±SEM of the pooled results of 3 independent experiments conducted in triplicate where activity was normalized as percentage of activity as related to the DMSO-treated control cells (“no-drug”) and non-stimulated (“NS”) cells.

FIG. 5A is a schematic showing an inflammasome multiprotein complex (adapted from Review InvivoGen, Inflammasomes, available at www.invivogen.com/review-inflammasome (2021)). In this illustrative inflammasome, the inflammasome complex contains a Nod-like receptor (NLR), the adapter apoptosis-associated speck-like (ASC) protein, and Caspase-1. As shown in this illustrative example of an inflammasome multiprotein complex, the NLR portion contains a pyrin domain (PYD) and nucleotide-binding and oligomerization domain (NACHT); the Caspase-1 portion contains a caspase recruitment domain (CARD) and p20 and p10 subunits; the ASC portion contains a PYD and CARD.

FIGS. 5B-5D are pictures showing the ability of probenecid analogs of the present technology (e.g., BT032) to inhibit the formation of the inflammasome complex following NLRP3 activation. NLRP3-deficient immortalized BMDMs reconstituted with ASC-cerulean (pseudocolor RED) and NLRP3-Flag were either not stimulated (FIG. 5B) or stimulated with NLRP3 agonist nigericin (3 μM) for 90 mins (FIG. 5C). ASC-cerulean macrophages were also pretreated with BT032 (350 μM) for 60 mins prior to nigericin challenge (FIG. 5D). Macrophages were fixed with 4% paraformaldehyde and imaged for the formation of inflammasome specks as identified by intense, punctate staining in the cytosol of cells. Cell nuclei were stained with DAP (4′,6′-diamindino-2-phenylindole; BLUE). Representative images shown are maximum intensity projections of 3D deconvoluted z stacks using ImageJ.

FIG. 5E is a chart showing the number of ASC specks detected per field (6-7 fields per sample) compared to the total number of cells/field as determined by staining nuclei. Data is represented as a percentage of ASC-specks per field of view for each treatment group as outlined in FIGS. 5B-5D.

FIG. 6A is a schematic of the mouse IP LPS challenge model for NLRP3 inflammation experiment.

FIG. 6B is a chart showing IL-1β levels (pg/mL) in mouse serum after administration of PBS or LPS (10 mg/kg) intraperitoneally (IP) with or without IP administration of BT032 (100 mg/kg) or BT132 (160 mg/kg) 1 hour prior to LPS administration.

FIG. 6C is a chart showing TNFα levels (pg/mL) in mouse serum after administration of PBS or LPS (10 mg/kg) IP with or without IP administration of BT032 (100 mg/kg) or BT132 (160 mg/kg) 1 hour prior to LPS administration.

FIG. 6D is a chart showing IL-1β levels (pg/mL) in mouse IP fluid after administration of PBS or LPS (10 mg/kg) intraperitoneally (IP) with or without IP administration of BT032 (100 mg/kg) or BT132 (160 mg/kg) 1 hour prior to LPS administration.

FIG. 6E is a chart showing TNFα levels (pg/mL) in mouse IP fluid after administration of PBS or LPS (10 mg/kg) intraperitoneally (IP) with or without IP administration of BT032 (100 mg/kg) or BT132 (160 mg/kg) 1 hour prior to LPS administration.

FIG. 7 is a chart showing the effects of treatment of macrophages with BT032, BT135, BT136, BT137, and BT159 (also known as BT052) on NLRP1 (L18-MDP)-induced inflammasome activation. Secreted IL-1β concentrations were measured by ELISA and are represented as the % maximal activation of untreated macrophages (“no drug”). Pooled results of 3 independent experiments were conducted in triplicate where activity was normalized as percentage of activity as related to the DMSO-treated control cells (“no drug”) and non-stimulated (“NS”) cells.

FIG. 8 is a chart showing the effects of treatment of either WT or NLRP3^{-/-} macrophages with BT032 on NLRP1 (L18-MDP)-induced inflammasome activation as measured by IL-1β secretion.

FIGS. 9A-9F are a series of panels showing ADS032 (also referred to herein as “BT032”) directly binds to NLRP1 and NLRP3. FIGS. 9A and 9B are chemical structures of ADS165 and ADS167. Recombinant NLRP3 (FIG. 9C) or NLRP1 (FIG. 9E) (2 μg) were co-incubated where indicated with ADS167 or MCC950 for 20 mins and then irradiated with UV 365 nm for a further 20 mins. ADS165 was then co-incubated for 20 mins and UV treated for 20 mins to cross-link associated compound and protein. Protein was separated by SDS-PAGE and immunoblotted (IB) with anti-PEG to visualize ADS165-linked protein. FIG. 9D: iBMDM cells stably expressing NLRP3-Flag were treated for 30 mins with ADS165, ADS167 (1 mM) or MCC955 (50 μM) for 30 mins, and irradiated with UV 365 nm for 30 mins. Cells were lysed with buffer and immunoprecipitation (IP) of NLRP3 performed with α-NLRP3 (D4D8T) antibody. Levels of precipitated (IP) and total cellular lysate (Input) NLRP3 were determined by immunoblotting with α-NLRP3 (Cryo-2) antibody. FIG. 9F: NLRP1-Flag was expressed in HEK293T cells (1×10⁶) for 20 h and treated with ADS165 (1 mM) for 30 mins where indicated, followed by UV 365 nm for a further 30 mins. Cells were lysed with lysis buffer and NLRP3 immunoprecipitated with α-Flag (M2) antibody. Proteins were separated by SDS-PAGE and ADS165-linked NLRP1 identified by immunoblot with a-PEG, while total precipitated NLRP1 visualized by immunoblot with α-NLRP1 and total cell lysate by α-Flag. Data shown is representative of three independent experiments.

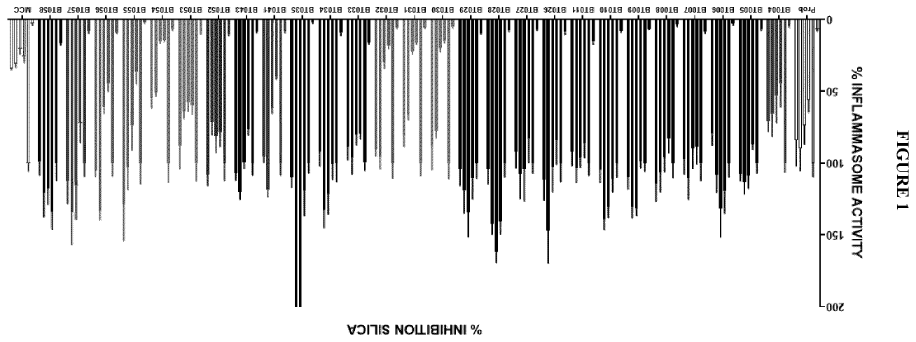

### DETAILED DESCRIPTION

### I. Definitions

The following terms are used herein, the definitions of which are provided for guidance.

In general, “substituted” refers to an organic group as defined below (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group is substituted with one or more substituents, unless otherwise specified. In some embodiments, a substituted group is substituted with 1, 2, 3, 4, 5, or 6 substituents. It will be understood by those of skill in the art that substituted groups of the present technology are chemically stable groups that allow isolation of the compounds in which they appear. Examples of substituent groups include: halogens (i.e., F, Cl, Br, and I); hydroxyls; alkoxy, alkenoxy, aryloxy, aralkyloxy, heterocyclyl, heterocyclylalkyl, heterocyclyloxy, and heterocyclylalkoxy groups; carbonyls (oxo); carboxylates; esters; urethanes; oximes; hydroxylamines; alkoxyamines; aralkoxyamines; thiols; sulfides; sulfoxides; sulfones; sulfonyls; sulfonamides; amines; N-oxides; azides; amides; ureas; amidines; guanidines; nitro groups; nitriles (i.e., CN); and the like.

Alkyl groups include straight chain and branched chain alkyl groups having (unless indicated otherwise) from 1 to 12 carbon atoms, and typically from 1 to 10 carbons or, in some embodiments, from 1 to 8, 1 to 6, or 1 to 4 carbon atoms. Alkyl groups may be substituted or unsubstituted. Examples of straight chain alkyl groups include groups such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. Representative substituted alkyl groups may be substituted one or more times with substituents such as those listed above, and include without limitation haloalkyl (e.g., trifluoromethyl), hydroxyalkyl, thioalkyl, aminoalkyl, alkylaminoalkyl, dialkylaminoalkyl, alkoxyalkyl, carboxyalkyl, and the like. In some embodiments the alkyl group is substituted with 1, 2, or 3 substituents.

Alkenyl groups include straight and branched chain alkyl groups as defined above, except that at least one double bond exists between two carbon atoms. Alkenyl groups may be substituted or unsubstituted. Alkenyl groups have from 2 to 12 carbon atoms, and typically from 2 to 10 carbons or, in some embodiments, from 2 to 8, 2 to 6, or 2 to 4 carbon atoms. In some embodiments, the alkenyl group has one, two, or three carbon-carbon double bonds. Examples include, but are not limited to vinyl, allyl, —CH═CH(CH₃), —CH═C(CH₃)₂, —C(CH₃)═CH₂, —C(CH₃)═CH(CH₃), —C(CH₂CH₃)═CH₂, among others. Representative substituted alkenyl groups may be mono-substituted or substituted more than once, such as, but not limited to, mono-, di- or tri-substituted with substituents such as those listed above.

Aralkyl groups are alkyl groups as defined above in which a hydrogen or carbon bond of an alkyl group is replaced with a bond to an aryl group as defined above. Aralkyl groups may be substituted or unsubstituted. In some embodiments, aralkyl groups contain 7 to 16 carbon atoms, 7 to 14 carbon atoms, or 7 to 10 carbon atoms. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl. Representative substituted aralkyl groups may be substituted one or more times with substituents such as those listed above.

Heteroalkyl groups and heteroalkenyl groups are, respectively, alkyl groups (as defined herein) and alkenyl groups (as defined herein) that include from 1 to 6 heteroatoms selected from N, O and S. It will be understood that each heteroatom present is bonded to at least one carbon atom within the heteroalkyl or heteroalkenyl group. In some embodiments the heteroaklyl or heteteroalkenyl groups include 1, 2, or 3 heteroatoms. Heteroalkyl and heteroalkenyl groups may be substituted or unsubstituted. Examples of heteroalkyl groups include but are not limited to CH₃CH₂OCH₂, CH₃NHCH₂, CH₃CH₂N(CH₃)CH₂, CH₃CH₂SCH₂, CH₃CH₂OCH₂CH₂OCH₂CH₂. Examples of heteroalkenyl groups include but are not limited to CH₂═CHOCH₂, CH₂═CHN(CH₃)CH₂, and CH₂═CHSCH₂. Representative substituted heteroalkyl or heteroalkeneyl groups may be substituted one or more times with substituents such as those listed above (e.g., 1, 2 or 3 times), and include without limitation haloheteroalkyl (e.g., trifluoromethyloxyethyl), carboxyalkylaminoalkyl, methyl acrylate and the like.

Cycloalkyl groups include mono-, bi- or tricyclic alkyl groups having from 3 to 12 carbon atoms in the ring(s), or, in some embodiments, 3 to 10, 3 to 8, or 3 to 4, 5, or 6 carbon atoms. Cycloalkyl groups may be substituted or unsubstituted. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups and fused rings, such as, but not limited to, bicyclo[2.1.1]hexane, adamantyl, decalinyl, and the like. Substituted cycloalkyl groups may be substituted one or more times with, non-hydrogen and non-carbon groups as defined above. However, substituted cycloalkyl groups also include rings that are substituted with straight or branched chain alkyl groups as defined above. Representative substituted cycloalkyl groups may be mono-substituted or substituted more than once, such as, but not limited to, 2,2-, 2,3-, 2,4- 2,5- or 2,6-disubstituted cyclohexyl groups, which may be substituted with substituents such as those listed above.

Cycloalkylalkyl groups are alkyl groups as defined above in which a hydrogen or carbon bond of an alkyl group is replaced with a bond to a cycloalkyl group as defined above. Cycloalkylalkyl groups may be substituted or unsubstituted. In some embodiments, cycloalkylalkyl groups have from 4 to 16 carbon atoms, 4 to 12 carbon atoms, and typically 4 to 10 carbon atoms. Substituted cycloalkylalkyl groups may be substituted at the alkyl, the cycloalkyl or both the alkyl and cycloalkyl portions of the group. Representative substituted cycloalkylalkyl groups may be mono-substituted or substituted more than once, such as, but not limited to, mono-, di- or tri-substituted with substituents such as those listed above.

Groups described herein having two or more points of attachment (i.e., divalent, trivalent, or polyvalent) within the compound of the present technology are designated by use of the suffix, “ene.” For example, divalent alkyl groups are alkylene groups, divalent cycloalkyl groups are cycloalkylene groups, divalent heteroalkyl groups are heteroalkylene groups, divalent alkenyl groups are alkenylene groups, and so forth. Substituted groups having a single point of attachment to the compound of the present technology are not referred to with the “ene” designation. Thus, e.g., chloroethyl is not referred to herein as chloroethylene.

The term “administering” a molecule to a subject means delivering the molecule to the subject or cells. “Administering” includes prophylactic administration of the composition (i.e., before the disease and/or one or more symptoms of the disease are detectable) and/or therapeutic administration of the composition (i.e., after the disease and/or one or more symptoms of the disease are detectable). The methods of the present technology include administering one or more compounds. If more than one compound is to be administered, the compounds may be administered together at substantially the same time, and/or administered at different times in any order. Also, the compounds of the present technology may be administered before, concomitantly with, and/or after administration of another type of drug or therapeutic procedure (e.g., surgery).

Use of the terms “comprising”, “including” or similar terms to describe or define an embodiment of a compound, composition or method having one or more elements shall be understood to also disclose embodiments “consisting” or “consisting essentially” of the elements and vice versa. In other words, disclosure of embodiments open to elements beyond those listed (“comprising”), also are to be understood to disclose embodiments which are closed to additional elements (“consisting”) or which may only include additional elements that do not materially affect the characteristics of the embodiment (“consisting essentially”). Likewise, embodiments consisting or consisting essentially of the listed elements shall be understood to disclose embodiments comprising those elements.

The term “conjugating,” and grammatical equivalents, when made in reference to conjugating a molecule of interest and a polymer means covalently linking the molecule of interest to the polymer. Linkage may be direct. Alternatively, linkage may be indirect via a linking group or moiety. Methods for conjugation to polymers are known in the art, including methods for conjugation to a polypeptide to produce a fusion protein (Pasut, Polymers 6:160-178 (2014); Medscape, Nanomedicine 5(6):915-935 (2010)). In some embodiments, the conjugate comprises probenecid conjugated to a PEG polymer.

As used herein, the terms “effective amount” or “therapeutically effective amount,” or “pharmaceutically effective amount” refer to a quantity sufficient to achieve a desired therapeutic and/or prophylactic effect, e.g., an amount which results in the full or partial amelioration of inflammasome-mediated dermatological disease or symptoms associated with inflammasome-mediated dermatological disease in a subject in need thereof. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In some embodiments, multiple doses are administered. Additionally or alternatively, in some embodiments, multiple therapeutic compositions or compounds are administered. In the methods described herein, the therapeutic compounds may be administered to a subject having one or more signs or symptoms of an inflammasome-mediated dermatological disease (e.g., elevated concentrations of inflammatory cytokines, such as IL-1β or IL-18 or MCP-1).

Pharmaceutically acceptable salts of compounds described herein are within the scope of the present technology and include acid or base addition salts which retain the desired pharmacological activity and are not biologically undesirable (e.g., the salt is not unduly toxic, allergenic, or irritating, and is bioavailable). When the compound of the present technology has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound of the present technology has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g. dicyclohexylamine, trimethylamine, triethylamine, pyridine, picoline, ethanolamine, diethanolamine, triethanolamine) or basic amino acids (e.g., arginine, lysine and ornithine). Such salts can be prepared in situ during isolation and purification of the compounds or by separately reacting the purified compound in its free base or free acid form with a suitable acid or base, respectively, and isolating the salt thus formed.

“Polymer” is a substance that has a molecular structure consisting chiefly or entirely of a large number of similar units bonded together. Polymers may occur naturally (e.g., cellulose, polypeptides, nucleotides sequences, etc.) or are artificial (e.g., plastics, resins, etc.). Polymers may be used as carriers of drugs to which they are conjugated (i.e., a polymer carrier), and may enhance the solubility of the conjugated drug, improve its pharmacokinetic profile, protect the drug against degradation, release the drug under certain conditions, such as change in pH or in the presence of enzymes, such as esterases, lipases or proteases. In addition, a targeting moiety or a solubilizer may also be introduced into the conjugate to boost its therapeutic index (Medscape, Nanomedicine 5(6):915-935(2010)). Polymers (including polymeric carriers) may also be utilized to restrict the distribution of the drug conjugated to it by, for example, preventing the conjugated drug from crossing into specific body compartments (e.g., from the gastrointestinal lumen to the underlying tissue). Polymers (including polymer carriers) are pharmaceutically acceptable and may be natural polymers and/or synthetic linear polymers, and include polyethylene glycol (PEG), dextran, periodate-oxidized dextran, polysialic acids (PSAs), hyaluronic acid (HA), dextrin, hydroxyethyl-starch (HES), poly(2-ethyl 2-oxazoline) (PEOZ), polyglutamic acid (PGA), polylactic acid (PLA), polylactic-co-glycolic (PLGA), poly(D,L-lactide-co-glycolide) (PLA/PLGA), poly(hydroxyalkylmethaacrylamide), polyglycerol, 25 polyamidoamine (PAMAM), polyethylenimine (PEI), and polypeptides.

As used herein, “inflammasome activation” means that an inflammasome is formed by association of a pattern recognition receptor such as NLRP3 with apoptosis associated speck-like protein containing a CARD (ASC) and a caspase-1 precursor due to a stimulating factor such as pathogen components and that caspase-1 is activated. Caspase-1 cleaves the pro-inflammatory cytokines IL-1β and IL-18 to their active forms and mediates a type of inflammatory cell death known as pyroptosis. Other intracellular pattern recognition receptors (PRRs), such as NLR family members, NLRP1 and NLRC4, non-NLR PRRs such as the double-stranded DNA (dsDNA) sensors absent in melanoma 2 (AIM2) and interferon-gamma-inducible protein 16 (IF116), are also capable of forming inflammasomes. The probenecid analogs of the present technology may inhibit inflammasome activation and thus inhibit the production of activated caspase-1. As a result, the probenecid analogs of the present technology may inhibit the release of one or more inflammatory cytokines such as MCP-1, IL-1β, IL-18, IL-6, and TNF-α, reduce ASC speck formation, and provide protection against inflammasome-mediated dermatological disease.

As used herein, “inflammasome-mediated dermatological disease or condition,” refers to skin-related genetic disorders such as NAIAD (NLRP1-associated auto-inflammation with arthritis and dyskeratosis), systemic sclerosis (also known as scleroderma), multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis. As demonstrated by the experimental examples presented herein, the probenecid analogs of the present technology (e.g., BT032) are effective as inhibitors of inflammasome activation and are effective in methods for preventing or treating inflammasome-mediated dermatological disease or condition. Therefore, because the probenecid analogs of the present technology are effective in such methods, a person of ordinary skill in the art would understand that the probenecid analogs of the present technology (e.g., BT032) are effective in methods for treating any inflammasome-mediated dermatological disease or condition and are not limited to the illustrative diseases/pathogens that cause inflammasome-mediated dermatological diseases or conditions listed herein.

As used herein, “to inhibit inflammasome activation” means to completely or partially inhibit the inflammasome activation by a stimulating factor. In other words, “to inhibit inflammasome activation” means to reduce the amount of produced activated caspase-1 or the amount of released inflammatory cytokine, such as MCP-1, IL-1β, IL-18, IL-6, and TNF-α, or the formation of ASC specks, with the compounds of the present technology as compared to a non-treated control.

Those of skill in the art will appreciate that compounds of the present technology may exhibit the phenomena of tautomerism, conformational isomerism, geometric isomerism and/or stereoisomerism. As the formula drawings within the specification and claims can represent only one of the possible tautomeric, conformational isomeric, stereoisomeric or geometric isomeric forms, it should be understood that the technology encompasses any tautomeric, conformational isomeric, stereoisomeric and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds disclosed herein include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the present technology.

“Tautomers” refers to isomeric forms of a compound that are in equilibrium with each other, and involve migration of at least one atom or group (e.g., a hydrogen atom) and at least one change in bond valence (e.g., between a single and double bond). The presence and concentrations of the isomeric forms will depend on the environment the compound is found in and may be different depending upon, for example, whether the compound is a solid or is in an organic or aqueous solution, what the temperature is, and whether an acid or base is present. For example, in aqueous solution, as shown below imines may be in equilibrium with enamines, which are referred to as tautomers of each other:

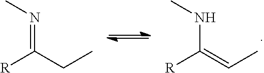

Similarly, those of skill in the art will be familiar with other tautomeric forms such as, e.g., keto/enol tautomers, keto/phenol tautomers, and the like. Because of the limits of representing compounds by structural formulas, it is to be understood that all chemical formulas of the compounds described herein represent all tautomeric forms of compounds and are within the scope of the present technology.

“Treating,” “treat,” “treated,” or “treatment” as used herein covers the treatment of a disease or disorder or condition described herein (e.g., inflammasome-mediated dermatological disease or an inflammasome-mediated dermatological condition), in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. Symptoms may be assessed by methods known in the art.

As used herein, “prevention” or “preventing” of a disorder or condition refers to a compound that, in a statistical sample, reduces the occurrence of the disorder or condition in the treated sample relative to a control sample, or delays the onset of one or more symptoms of the disorder or condition relative to the control sample.

It is also to be appreciated that the various modes of treatment or prevention of medical diseases and conditions as described are intended to mean “substantial,” which includes total but also less than total treatment or prevention, and wherein some biologically or medically relevant result is achieved.

As used herein, the terms “subject,” “individual,” or “patient” can be an individual organism, a vertebrate, a mammal, or a human. “Mammal” includes a human, non-human primate, murine (e.g., mouse, rat, guinea pig, hamster), ovine, bovine, ruminant, lagomorph, porcine, caprine, equine, canine, feline, aves, etc. In some embodiments, the mammal is murine. In some embodiments, the mammal is human.

A subject “in need” of treatment according to the methods and/or compositions of the present technology includes a subject that is “suffering” from an inflammasome-mediated dermatological disease or condition (i.e., a subject that is experiencing and/or exhibiting one or more clinical and/or subclinical symptoms of an inflammasome-mediated dermatological disease or condition), and a subject “at risk” of an inflammasome-mediated dermatological disease or condition. A subject “in need” of treatment includes animal models of inflammasome-mediated dermatological disease or condition. Subject “at risk” of inflammasome-mediated dermatological disease or condition refers to a subject that is not currently exhibiting inflammasome-mediated dermatological disease or condition symptoms and is predisposed to expressing one or more symptoms of the disease or condition. This predisposition may be based on family history, genetic factors, environmental factors such as exposure to detrimental compounds present in the environment, etc. It is not intended that the present technology be limited to any particular signs or symptoms. Thus, it is intended that the present technology encompass subjects that are experiencing any range of disease or condition, from sub-clinical symptoms to full-blown inflammasome-mediated dermatological disease, wherein the subject exhibits at least one of the indicia (e.g., signs and symptoms) associated with the inflammasome-mediated dermatological disease or condition.

### II. General

Activation of certain NLRs (NLRP1, NLRP3, and NLRC4) leads to assembly of inflammasomes, which are large macromolecular signaling complexes that control the proteolytic activation of proinflammatory cytokines of the IL-1 family (e.g., IL-1β and IL-18) in response to any array of stimuli such as pathogens (e.g., viral or bacterial infections), environmental irritants, and endogenous danger signals. Growing evidence indicates that the inflammasome plays a key role in the pathogenesis of dermatological diseases.

In one aspect, the present technology provides methods, compounds, and compositions for treating, preventing, or ameliorating inflammasome-mediated dermatological disease. In some embodiments, the inflammasome-mediated dermatological disease comprises skin-related genetic disorders such as NAIAD (NLRP1-associated auto-inflammation with arthritis and dyskeratosis), systemic sclerosis (also known as scleroderma), multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

As demonstrated by the experimental examples presented herein, the probenecid analogs of the present technology (e.g., BT032, BT132, BT135, BT136, BT137, and BT159) are effective as inhibitors of inflammasome activation and are effective in methods for preventing or treating inflammasome-mediated dermatological diseases or conditions. The experimental examples also demonstrate that the probenecid analogs of the present technology are effective in methods for preventing or treating inflammasome-mediated dermatological diseases or conditions. Therefore, because the probenecid analogs of the present technology are effective in such methods, a person of ordinary skill in the art would understand that the probenecid analogs of the present technology (e.g., BT032, BT132, BT135, BT136, BT137, and BT159) are effective in methods for treating any inflammasome-mediated dermatological disease and are not limited to the illustrative diseases/pathogens that cause inflammasome-mediated dermatological disease listed herein.

In some embodiments, treating or preventing and inflammasome-mediated dermatological disease comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject. In the case of recurrent respiratory papillomatosis, signs and symptoms include hoarseness, stridor, dysphonia, aphonia, chronic cough, dysphagia, dyspnea, choking episodes, acute respiratory distress, recurrent pneumonia, and bronchiectasis.

### III. Compounds of the Present Technology

The present technology provides compositions for treating inflammasome-mediated dermatological disease. In some embodiments, the present technology provides compositions for treating inflammasome-mediated dermatological disease and conditions.

In some embodiments, the present technology discloses a probenecid analog defined by Formula I and pharmaceutically acceptable salts thereof:

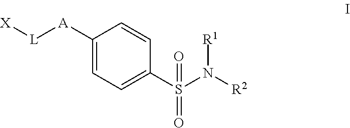

tautomers thereof and/or pharmaceutically acceptable salts thereof;
wherein
A is absent, or is selected from the group consisting of C(O)N(R³), phenylene, oxazolylene, thiazolylene, piperidinylene and

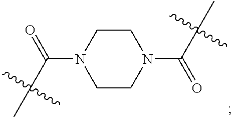 [SMILES: *C(=O)N1CCN(C(*)=O)CC1]

L is absent or C₁₋₁₀ alkylene;
X is H, CHO, COOH, C(O)NR⁴R⁵, COOR⁶, NH₂ or NHR;
R is 2-chloropyrimidin-4-yl;
R¹ and R² are independently a substituted or unsubstituted C₁₋₆ alkyl group, or one of R¹ and R² is H, and the other is cyclohexyl-NH—C(O), or R¹ and R² together are a C₄₋₆ alkylene group and form a 5-, 6-, or 7-member ring with the nitrogen to which they are attached, said ring optionally substituted with a phenyl group;
R³ and R⁴ are independently selected from H or a C₁₋₆ alkyl group;
R⁵ is selected from H, PEG, or a C₁₋₆ alkyl group; and
R⁶ is selected from a substituted or unsubstituted C₁₋₁₀ alkyl, C₂₋₁₀ alkenyl, or C₇₋₁₄ aralkyl group.

In some embodiments of the compounds of Formula I, A may be absent. In some embodiments, A may be C(O)N(R³), wherein R³ is H or a C₁₋₆ alkyl group. In some such embodiments R³ may be H or methyl. In some embodiments, A may be phenylene, oxazolylene, thiazolylene, piperidinylene or

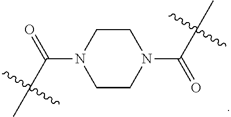 [SMILES: *C(=O)N1CCN(C(*)=O)CC1]

For example, A may be phenylene.

In some embodiments of compounds of Formula I, L may be absent. In some embodiments L may be a C₁₋₁₀ alkylene, for example a C₁, C₂, C₃, C₄, C₅, C₆, C₇, C₈, C₉, or C₁₀ alkylene or a range between and including any two of the foregoing values of L.

In some embodiments of compounds of Formula I, X may be H. In some embodiments, X may be COOH. In some embodiments, X may be COOR⁶, where R⁶ may be defined as herein. For example, R⁶ may be a substituted or unsubstituted C₁₋₁₀ alkyl group, or a C₁₋₆ alkyl group, e.g., a substituted or unsubstituted methyl, ethyl or propyl group, e.g., substituted with one, two, or three substituents. In any embodiments, the one, two, or three substitutents may be selected from, e.g., F, OH, NH₂, NH(C₁₋₄ alkyl), or NH(C₁₋₄ alkyl). In any embodiments, R⁶ may be methyl, ethyl, 2-dimethylaminoethyl, 2-hydroxyethyl, or 2,3-dihydroxypropyl. R⁶ may be a substituted or unsubstituted C₂₋₁₀ alkenyl group, or a C₂₋₆ alkenyl group, e.g., a substituted or unsubstituted allyl group. R⁶ may be a substituted or unsubstituted C₇₋₁₄ aralkyl group or a substituted or unsubstituted C₇₋₁₀ aralkyl group, e.g., a substituted or unsubstituted benzyl or phenethyl group. In some embodiments X may be NH₂ or X may be NHR where R is R is 2-chloropyrimidin-4-yl. In some embodiments, X may be C(O)NR⁴R⁵ where R⁴ and R⁵ may be as defined herein. For example R⁴ may be H, or R⁴ may be a C₁₋₆ alkyl group. In some embodiments, R⁵ may be H. In some embodiments, R⁵ may be a C₁₋₆ alkyl group. In some embodiments, R⁵ may be a polyethylene glycol (PEG).

PEG may have any suitable geometry (linear, branched, multi-arm) and any suitable average molecular weight. In some embodiments, the PEG is a linear PEG. In some embodiments, the PEG may have an average molecular weight in the range of about 100 Da to about 40 kDa. (Unless otherwise indicated, “average molecular weight” means weight average molecular weight.) In some embodiments, the average molecular weight of the polymer is about 100 Da, 200 Da, 300 Da, 400 Da, 500 Da, 550 Da, 600 Da, 700 Da, 800 Da, 900 Da, 1 kDa, 1.5 kDa, 2 kDa, 3 kDa, 4 kDa, 5 kDa, 7.5 kDa, 10 kDa, 15 kDa, 20 kDa, 25 kDa, 30 kDa, 40 kDa, or any range between and including two of these values. For example, the PEG may have an average molecular weight in the range of about 500 Da to about 2 or to about 3 kDa.

In some embodiments, the PEG may be functionalized at one or more of its termini with amine (NH₂) and/or aldehyde (CHO) groups and include linear mono-amines and mono-aldehydes, linear bi-amines and bi-aldehydes, multi-arm-amines and multi-arm-aldehydes, branched mono-, bi- and multi-armed-amines and aldehydes and multi-arm-forked-amines and aldehydes. The PEG may terminate in a hydroxyl or in a C₁₋₆ ether, e.g., a methyl or ethyl ether. In some embodiments the PEG may be functionalized with an amine at one terminus and a hydroxyl or C₁₋₆ ether at another terminus. In some such embodiments, the PEG is a linear PEG.

In some embodiments of compounds of Formula I, each of R¹ and R² may independently be a C₁₋₆ alkyl group. The latter may be optionally substituted, e.g., with one or more (e.g., 1, 2, or 3) F, OH, CF₃, C₃₋₇ cycloalkyl group or SO₂-alkyl. In some embodiments, R¹ and R² together may be a C₄₋₆ alkylene group and form a 5-, 6-, or 7-member ring with the nitrogen to which they are attached. Thus, R¹ and R² together may be a C₄₋₆ alkylene group and form a pyrrolidine, piperidine, or azepane, each of which may be optionally substituted with a phenyl group. In some embodiments, one of R¹ and R² is H, and the other is cyclohexyl-NH—C(O).

In some embodiments of compounds of Formula I, A is absent, or is selected from the group consisting of C(O)N(R³), phenylene, oxazolylene, thiazolylene, and piperidinylene, L is absent or C₁₋₁₀ alkylene; X is H, COOH, or NH₂; R¹ and R² are independently a substituted or unsubstituted C₁₋₆ alkyl group; and R³ is selected from H or a C₁₋₆ alkyl group. In some embodiments, A may be absent and L may be C₃₋₁₀ alkylene. In some embodiments, A may be phenylene, oxazolylene, thiazolylene, or piperidinylene, and L is absent or a C₁₋₅ alkylene. In some embodiments, X may be COOH or NH₂.

It will be appreciated by those of skill in the art that disclosure of any compound herein, including compounds of Formula I and other probenecid analogs also discloses tautomers thereof, and/or pharmaceutically acceptable salts thereof. In some embodiments, the present technology discloses a probenecid analog defined by Formula Ia (BT004):

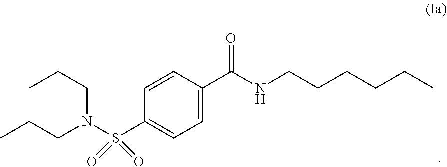 [SMILES: CCCCCCNC(=O)c1ccc(S(=O)(=O)N(CCC)CCC)cc1]

In some embodiments, the present technology discloses a probenecid analog defined by Formula Ib (BT005):

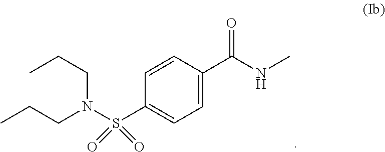 [SMILES: CCCN(CCC)S(=O)(=O)c1ccc(C(=O)NC)cc1]

In some embodiments, the present technology discloses a probenecid analog defined by Formula Ic (BT006):

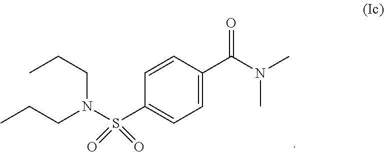 [SMILES: CCCN(CCC)S(=O)(=O)c1ccc(C(=O)N(C)C)cc1]

In some embodiments, the present technology discloses a probenecid analog defined by Formula Id (BT007):

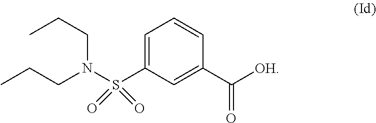 [SMILES: CCCN(CCC)S(=O)(=O)c1cccc(C(=O)O)c1]

In some embodiments, the present technology discloses a probenecid analog defined by Formula Je (BT008):

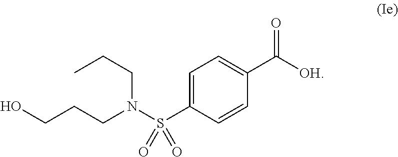 [SMILES: CCCN(CCCO)S(=O)(=O)c1ccc(C(=O)O)cc1]

In some embodiments, the present technology discloses a probenecid analog defined by Formula If (BT009):

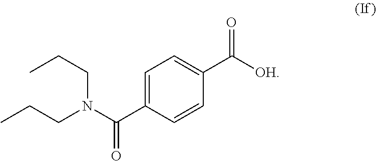 [SMILES: CCCN(CCC)C(=O)c1ccc(C(=O)O)cc1]

In some embodiments, the present technology discloses a probenecid analog defined by Formula Ig (BTO10):

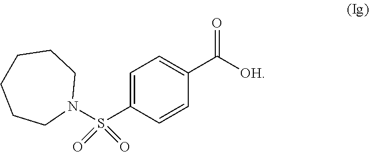 [SMILES: O=C(O)c1ccc(S(=O)(=O)N2CCCCCC2)cc1]

In some embodiments, the present technology discloses a probenecid analog defined by Formula Ih (BTO11):

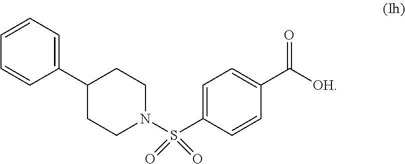 [SMILES: O=C(O)c1ccc(S(=O)(=O)N2CCC(c3ccccc3)CC2)cc1]

In some embodiments, the present technology discloses a probenecid analog defined by Formula II (BT026):

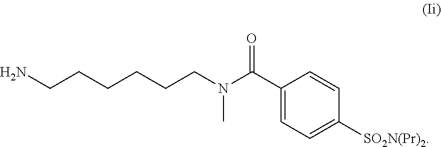

In some embodiments, the present technology discloses a probenecid analog defined by Formula Ij (BT027):

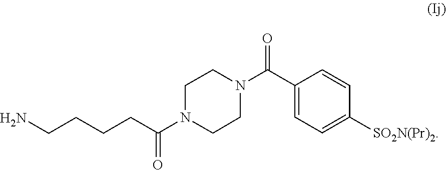

In some embodiments, the present technology discloses a probenecid analog defined by Formula Ik (BT028):

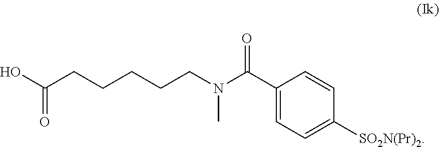

In some embodiments, the present technology discloses a probenecid analog defined by Formula II (BT029):

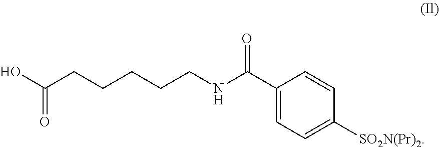

In some embodiments, the present technology discloses a probenecid analog defined by Formula Im (BT030):

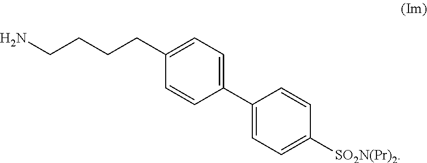

In some embodiments, the present technology discloses a probenecid analog defined by Formula In (BT031):

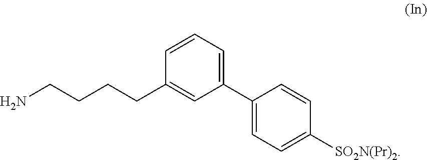

In some embodiments, the present technology discloses a probenecid analog defined by Formula Io (BT032):

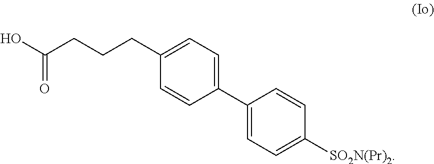

In some embodiments, the present technology discloses a probenecid analog defined by Formula Ip (BT033):

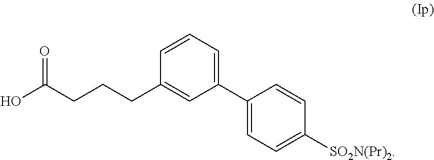

In some embodiments, the present technology discloses a probenecid analog defined by Formula Iq (BT034):

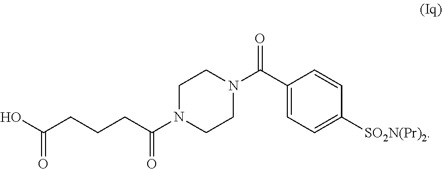

In some embodiments, the present technology discloses a probenecid analog defined by Formula Is (BT041):

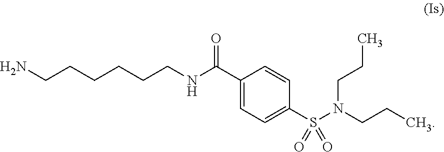 [SMILES: CCCN(CCC)S(=O)(=O)c1ccc(C(=O)NCCCCCCN)cc1]

In some embodiments, the present technology discloses a probenecid analog defined by Formula It (BT043):

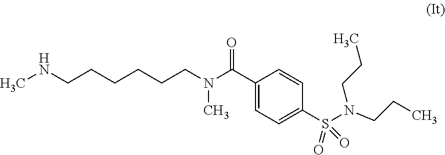 [SMILES: CCCN(CCC)S(=O)(=O)c1ccc(C(=O)N(C)CCCCCCNC)cc1]

In some embodiments, the present technology discloses a probenecid analog defined by Formula Iu (BT052, also known as BT159):

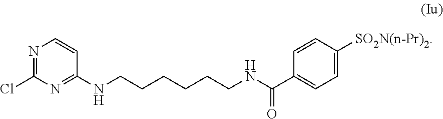

In some embodiments, the present technology discloses a probenecid analog defined by Formula IV (BT053):

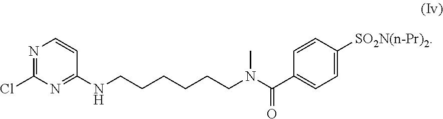

In some embodiments, the present technology discloses a probenecid analog defined by Formula Iw (BT054):

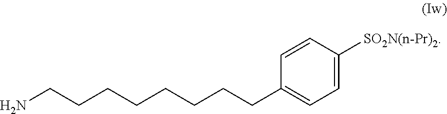

In some embodiments, the present technology discloses a probenecid analog defined by Formula Ix (BT055):

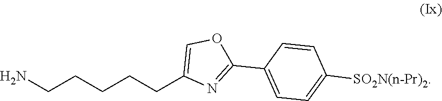

In some embodiments, the present technology discloses a probenecid analog defined by Formula Iy (BT056):

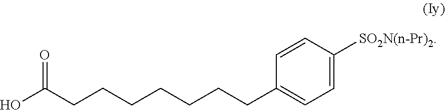

In some embodiments, the present technology discloses a probenecid analog defined by Formula Iz (BT057):

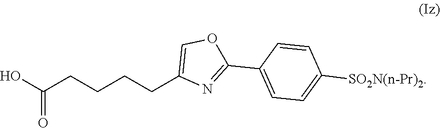

In some embodiments, the present technology discloses a probenecid analog defined by Formula Iaa (BT058):

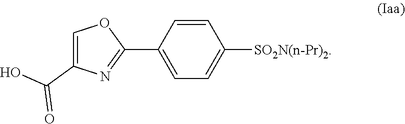

In some embodiments, the present technology discloses a probenecid analog defined by Formula Iab (BT132):

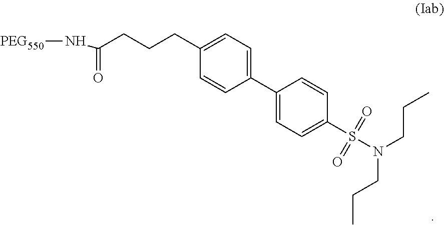

In some embodiments, the present technology discloses a probenecid analog defined by Formula Iac (BT133):

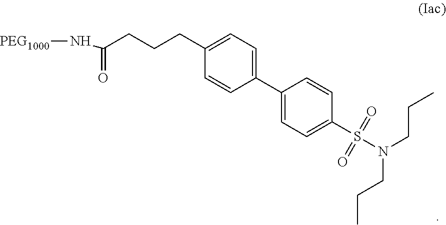

In some embodiments, the present technology discloses a probenecid analog defined by Formula lad (BT134):

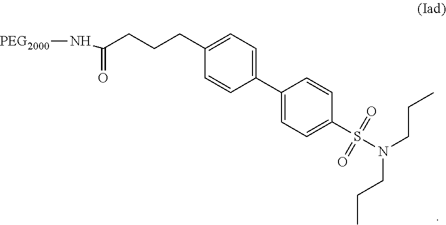

In some embodiments, the present technology discloses a probenecid analog defined by Formula Iae (BT135):

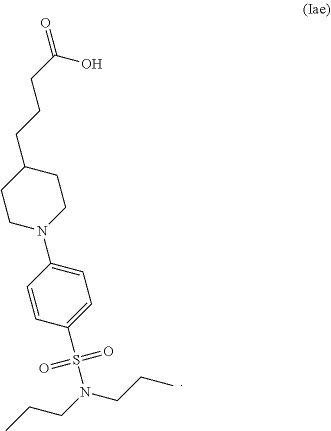 [SMILES: CCCN(CCC)S(=O)(=O)c1ccc(N2CCC(CCCC(=O)O)CC2)cc1]

In some embodiments, the present technology discloses a probenecid analog defined by Formula Iaf (BT136):

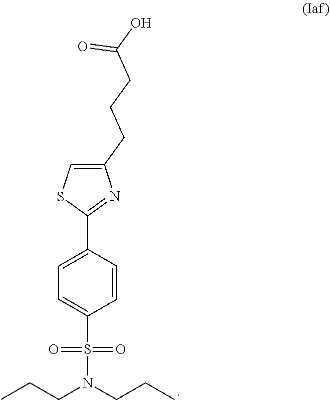 [SMILES: CCCN(CCC)S(=O)(=O)c1ccc(-c2nc(CCCC(=O)O)cs2)cc1]

In some embodiments, the present technology discloses a probenecid analog defined by Formula Iag (BT137):

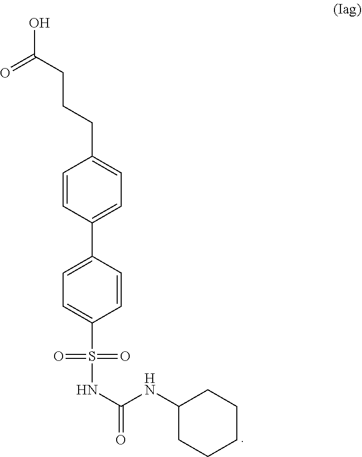 [SMILES: O=C(O)CCCc1ccc(-c2ccc(S(=O)(=O)NC(=O)NC3CCCCC3)cc2)cc1]

In some embodiments, the present technology discloses a probenecid analog defined by Formula Iah (BT138):

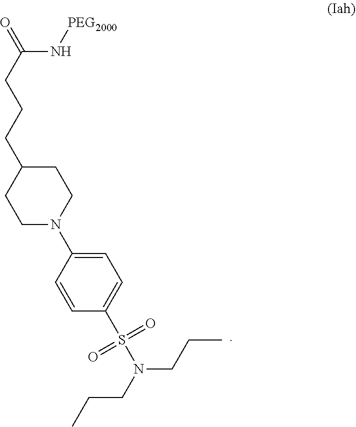

In some embodiments, the present technology discloses a probenecid analog defined by Formula Iai (BT139):

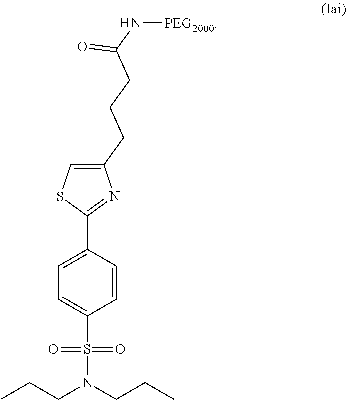

In some embodiments, the present technology discloses a probenecid analog defined by Formula Iaj (BT140):

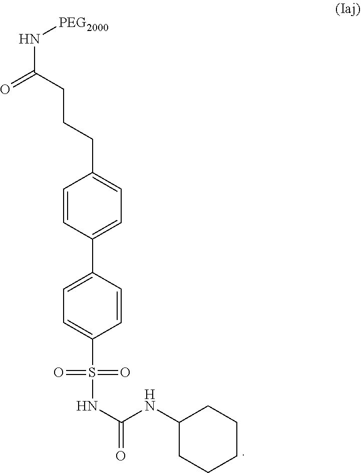

In some embodiments, the present technology discloses a probenecid analog defined by Formula Iak (BT168; also referred to as BT032-OEt):

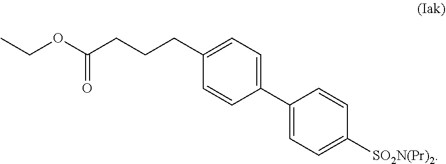

In some embodiments, the present technology discloses a probenecid analog defined by Formula Ial (BT169; also referred to as BT032-OGly):

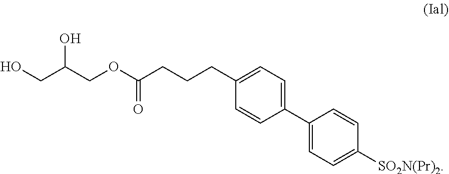

In some embodiments, the present technology discloses a probenecid analog defined by Formula lam (BT170; also referred to as BT032-ODMEA):

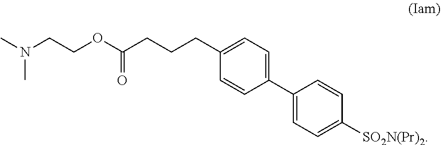

In some embodiments, the present technology discloses a probenecid analog defined by Formula Ian (BT160):

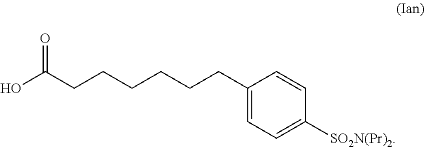

In another aspect, the present disclosure provides compounds of Formula II:

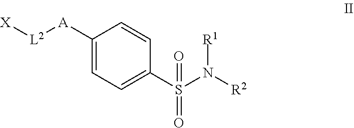

tautomers thereof and/or pharmaceutically acceptable salts thereof;
wherein
A is absent, or is selected from the group consisting of C(O)N(R³), phenylene, oxazolylene, thiazolylene, piperidinylene, and

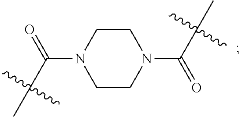 [SMILES: *C(=O)N1CCN(C(*)=O)CC1]

L² is absent or is a C₁₋₁₂ alkylene or C₁₋₁₂ heteroalkylene group, or a peptide comprising 2-10 amino acid residues;
X is H, CHO, COOH, C(O)NR⁴R⁵, COOR⁶, NH₂ or NHR;
R is 2-chloropyrimidin-4-yl;
R¹ and R² are independently a substituted or unsubstituted C₁₋₆ alkyl group, or one of
R¹ and R² is H, and the other is cyclohexyl-NH—C(O), or R¹ and R² together are a C₄₋₆ alkylene group and form a 5-, 6-, or 7-member ring with the nitrogen to which they are attached, said ring optionally substituted with a phenyl group;
R³ and R⁴ are independently selected from H or a C₁₋₆ alkyl group;
R⁵ is selected from H, a polymer carrier, or a C₁₋₆ alkyl group, wherein the polymer carrier is pharmaceutically acceptable polymer; and
R⁶ is selected from a substituted or unsubstituted C₁₋₁₀ alkyl, C₂₋₁₀ alkenyl, or C₇₋₁₄ aralkyl group.

In the present aspect, probenecid (or analogs thereof, e.g., as defined in Formula II) is attached to a polymer carrier via a linker L². In some embodiments, the linker can serve as a spacer to distance the probenecid analog and the polymer in order to avoid interference, with, for example, binding capabilities. The linker comprises one or more atoms, e.g., one or more atoms selected from C, N, or O. In some such embodiments the linker may further comprise one or more H atoms, e.g., NH, N(CH₃), or CH₂.

In some embodiments, the linker is a biodegradable linker. In some embodiments, the biodegradable linker comprises an oligopeptide having from 2 to 10 amino acid residues. The residues may be selected from the naturally occurring amino acids.

In some embodiments, the linker comprises a substituted or unsubstituted C₁-C_{z} alkylene, cycloalkylene, cycloalkylalkylene, heteroalkylene, alkenylene, or heteroalkenylene group, wherein z may be any integer from 1 to 12, i.e., 1, 2, 3, 4, 5, 6, 7, 8, 9, 10, 11 or 12. For example, the linker may comprise a C1-Cz fluoroalkyl group where one or more of the hydrogen atoms are fluorine atoms, such as 1, 2 or 3 or more fluorines. In some embodiments, L² is a heteroalkylene containing one or two NH groups, including but not limited to (C₁-C₁₀ alkylene)-NH (e.g., CH₂CH₂NH, CH₂CH₂CH₂NH, CH₂CH₂CH₂CH₂NH, CH₂CH(CH₃)CH(CH₃)CH₂NH), (Cₙ alkylene)NH(Cₚ alkylene) where n, p are independently an integer from 1-10, but n+p does not exceed 10 (e.g., CH₂CH₂CH₂NH CH₂CH₂), NH—(C₁-C₁₀ alkylene)NH (e.g., NH(CH₂)₅NH, NH(CH₂)₆NH, NH(CH₂)₈NH), or NH(Cₙ alkylene)NH(Cₚ alkylene) where n and p are integers as defined previously (e.g., NHCH₂CH₂CH₂NH CH₂CH₂, NH(CH₂)₆NHCH₂). In some embodiments, L² is a heteroalkylene that contains one or two oxygen atoms, including but not limited to (C₁-C₁₀ alkylene)-O (e.g., CH₂CH₂O, CH₂CH₂CH₂O, CH₂CH₂CH₂CH₂O, CH₂CH(CH₃)CH(CH₃)CH₂O), (Cₙ alkylene)O(Cₚ alkylene) where n, p are independently an integer from 1-10, but n+p does not exceed 10 (e.g., CH₂CH₂CH₂OCH₂CH₂), O—(C₁-C₁₀ alkylene)O (e.g., O(CH₂)₅O, O(CH₂)₆O, O(CH₂)₈O), or O(Cₙ alkylene)O(Cₚ alkylene) where n and p are integers as defined previously (e.g., OCH₂CH₂CH₂OCH₂CH₂, O(CH₂)₆OCH₂). In some embodiments, L² is a heteroalkylene containing an O and an NH group, including but not limited to NH—(C₁-C₁₀ alkylene)O, (e.g., NH(CH₂)₅O, NH(CH₂)₆O, NH(CH₂)₈O), or NH(Cₙ alkylene)O(Cₚ alkylene) where n and p are integers as defined previously (e.g., NHCH₂CH₂OCH₂CH₂, O(CH₂)₆NHCH₂).

In some embodiments, the polymer carrier is selected from the group consisting of PEG, dextran, periodate-oxidized dextran, polysialic acids (PSAs), hyaluronic acid (HA), dextrin, hydroxyethyl-starch (HES), poly(2-ethyl 2-oxazoline) (PEOZ), polyglutamic acid (PGA), polylactic acid (PLA), polylactic-co-glycolic (PLGA), poly(D,L-lactide-co-glycolide) (PLA/PLGA), poly(hydroxyalkylmethaacrylamide), polyglycerol, 25 polyamidoamine (PAMAM), polyethylenimine (PEI), and polypeptides (i.e., comprising alpha-amino acid residues). The polymer may have any suitable weight average molecular weight, e.g., from about 100 Da to about 40 kDa. In some embodiments, the average molecular weight of the polymer is about 100 Da, 200 Da, 300 Da, 400 Da, 500 Da, 550 Da, 600 Da, 700 Da, 800 Da, 900 Da, 1 kDa, 1.5 kDa, 2 kDa, 3 kDa, 4 kDa, 5 kDa, 7.5 kDa, 10 kDa, 15 kDa, 20 kDa, 25 kDa, 30 kDa, 40 kDa, or any range between and including two of these values. In some embodiments, the polymer carrier is PEG and may have the structure disclosed herein above.

In addition, variables A, X, R, R¹, R², R³, R⁴, R⁵, R⁶, may also have any of the values disclosed herein, e.g., any of the values disclosed with respect to Formula I, as all such embodiments are intended for use with Formula II as well.

The probenecid-polymer conjugates may be prepared using standard techniques known in the art. In some embodiments, a difunctional linker containing at least two functional groups containing heteroatoms selected from N, O, and S in which one of the functional groups is protected may be conjugated using standard ester, thioester and amide bond forming technology. For example, a diamino-alkylene linker in which one of the amino groups is protected by a urethane protecting group (e.g., Boc. Cbz, etc.) may be coupled to probenecid in the presence of a coupling agent (e.g., DCC, EDC/HOBt, etc.). Alternatively, an active ester, mixed anhydride or acid halide derivative of probenecid may be prepared and reacted with the mono-protected diamine. (See, for example, Bodanszky, M. & Bodanszky, A., The Practice of Peptide Synthesis, Springer-Verlag, New York, 1984.) The protecting group may be removed and the free amine reacted with an aldehyde derivative of the polymer under reducing conditions to provide the conjugate. Similarly, a linker with a protected aldehyde (e.g., 1,1-dimethoxy) and an amine may be coupled to the probenecid, deprotected to form the aldehyde and subjected to reductive amination with an amino-bearing polymer to form the conjugate. Variations of these schemes using α,ω-carboxy amines, α,ω-aminoalcohols, α,ω-carboxyalcohols, α,ω-aminothiols, and the like to link probenecid and the polymer will be readily understood by those of skill in the art.

### IV. Use of the Compositions of the Present Technology

The present technology provides methods for treating, preventing, or ameliorating inflammasome-mediated dermatological disease or conditions in a mammalian subject in need thereof, comprising administering to the subject a therapeutically effective amount of one or more probenecid analogs of the present technology. In some embodiments, the inflammasome-mediated dermatological disease or condition is a skin-related genetic disorder such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis. In some embodiments, the probenecid analog is a compound of Formula I and pharmaceutically acceptable salts thereof, including a compound of any one of Formulas Ia-Iaj. In some embodiments, the probenecid analog is a compound of Formulas Io (BT032), Jab (BT132), Iac (BT133), Iae (BT135), Iaf (BT136), or Jag (BT137). In a further embodiment, the probenecid analogs of the present technology reduce pro-inflammatory cytokine production in the skin. In some embodiments, the probenecid analogs of the present technology diminish IL-1β secretion.

As demonstrated by the experimental examples presented herein, the probenecid analogs of the present technology (e.g., BT032) are effective as inhibitors of inflammasome activation and are effective in methods for preventing or treating inflammasome-mediated dermatological disease or conditions. Therefore, because the probenecid analogs of the present technology are effective in such methods, a person of ordinary skill in the art would understand that the probenecid analogs of the present technology (e.g., BT032) are effective in methods for treating any inflammasome-mediated dermatological disease or condition and are not limited to the illustrative diseases/pathogens that cause inflammasome-mediated dermatological disease or conditions listed herein.

### V. Combination Therapies

In some embodiments, the probenecid analogs of the present technology may be combined with one or more additional therapeutic agents for the prevention, amelioration, or treatment of inflammasome-mediated dermatological diseases or conditions.

In one embodiment, an additional therapeutic agent is administered to a subject in combination with a probenecid analog of the present technology (e.g., a compound of Formula I, including but not limited to Formulas Io (BT032), Jab (BT132), Iac (BT133), Iae (BT135), Iaf (BT136), or Jag (BT137)) such that a synergistic therapeutic effect is produced.

In some embodiments, the probenecid analogs of the present technology (e.g., a compound of Formula I, including but not limited to Formulas Io (BT032), Jab (BT132), Iac (BT133), Iae (BT135), Iaf (BT136), or Jag (BT137)) are combined with one or more compounds for the treatment or prevention of inflammasome-mediated dermatological diseases or conditions including, but not limited to, skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

As demonstrated by the experimental examples presented herein, the probenecid analogs of the present technology (e.g., BT032) are effective as inhibitors of inflammasome activation and are effective in methods for preventing or treating inflammasome-mediated dermatological diseases or conditions. Therefore, because the probenecid analogs of the present technology are effective in such methods, a person of ordinary skill in the art would understand that the probenecid analogs of the present technology (e.g., BT032) are effective in methods for treating any inflammasome-mediated dermatological diseases or conditions and are not limited to the illustrative diseases/pathogens that cause inflammasome-mediated dermatological diseases or conditions listed herein.

The multiple therapeutic agents may be administered in any order or even simultaneously. If simultaneously, the multiple therapeutic agents may be provided in a single, unified form, or in multiple forms (by way of example only, either as a single formulation or as two separate formulations). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

In some embodiments, the methods of the present technology may further comprise administering one or more corticosteroids, immunosuppressants, nonsteroidal anti-inflammatory drugs (NSAIDS), IL-1β inhibitors, MCC950, and canakinumab, or any combination thereof.

### VI. Modes of Administration

Any method known to those in the art for contacting a cell, organ, or tissue with compounds of the present technology may be employed. Suitable methods include in vitro, ex vivo, or in vivo methods.

In vitro methods typically include cultured samples. For example, a cell can be placed in a reservoir (e.g., tissue culture plate), and incubated with a compound under appropriate conditions suitable for obtaining the desired result. Suitable incubation conditions can be readily determined by those skilled in the art.

Ex vivo methods typically include cells, organs or tissues removed from a mammal, such as a human. The cells, organs or tissues can, for example, be incubated with the compound under appropriate conditions. The contacted cells, organs or tissues are typically returned to the donor, placed in a recipient, or stored for future use. Thus, the compound is generally in a pharmaceutically acceptable carrier.

In vivo methods typically include the administration of a compound of the present technology to a mammal such as a human. When used in vivo for therapy, a compound of the present technology is administered to a mammal in an amount effective to obtain the desired result, e.g., of treating the mammal. The effective amount is determined during pre-clinical trials and clinical trials by methods familiar to physicians and clinicians. The dose and dosage regimen will depend upon the degree of the disease or condition in the subject, the characteristics of the particular compound of the present technology used, e.g., its therapeutic index, the subject, and the subject's history.

An effective amount of a compound of the present technology useful in the present methods, such as in a pharmaceutical composition or medicament, may be administered to a mammal in need thereof by any of a number of well-known methods for administering pharmaceutical compositions or medicaments. The compounds of the present technology may be administered systemically or locally.

The compounds of the present technology described herein can be incorporated into pharmaceutical compositions for administration, singly or in combination, to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

In some embodiments, the pharmaceutical compositions of the present disclosure contain a pharmaceutically acceptable carrier and/or excipient suitable for rendering the compound or mixture administrable orally as a tablet, capsule or pill, or parenterally, intravenously, topically (e.g., via a gel, cream, ointment, spray, or foam), intradermally, intramuscularly, intracutaneously, subcutaneously, or transdermally.

Pharmaceutical compositions are typically formulated to be compatible with the intended route of administration. Administering the pharmaceutical composition of the present disclosure may be accomplished by any means known to the skilled artisan. Routes of administration include, but are not limited to, parenteral, intranasal/respiratory (e.g., inhalation), intravenous, intramuscular, intradermal, intraperitoneal, intratracheal, intracutaneous, subcutaneous, oral, transdermal (topical, e.g., via a gel, cream, ointment, spray, or foam), sublingual, ocular, vaginal, rectal, and transmucosal administration. Systemic routes include oral and parenteral. Several types of devices are regularly used for administration by inhalation. These types of devices include metered dose inhalers (MDI), breath-actuated MDI, dry powder inhaler (DPI), spacer/holding chambers in combination with MDI, and nebulizers.

For oral administration, the compounds can be formulated readily by combining the active compound(s) with pharmaceutically acceptable carriers well known in the art. Such carriers enable the compounds of the disclosure to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present disclosure may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

In some embodiments, the compounds for use according to the present disclosure may be formulated for intra-alveolar administration, For example, in some embodiments, the compounds for use according to the present disclosure may be administered during a bronchoscopy procedure or microendoscopy procedure, which delivers the compound to alveolar spaces (i.e., microdosing in the lung).

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In some embodiments, administration is topical and/or at the luminal surface of the tissue to be treated. “Topical” administration of a composition means contacting the composition with the skin. “Luminal surface” refers to the inner open space or cavity of a tubular organ, such as the interior central space in an artery or vein through which blood flows; the interior of the gastrointestinal tract; the pathways of the bronchi in the lungs; the interior of renal tubules and urinary collecting ducts; the pathways of the female genital tract, starting with a single pathway of the vagina, splitting up in two lumina in the uterus, both of which continue through the fallopian tubes.

In some embodiments, the compounds of the present technology are administered topically and/or at a luminal surface of the target tissue. This is advantageous to reduce potential systemic toxic side effects of the compounds.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the disclosure is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

### EXPERIMENTAL EXAMPLES

The present technology is further illustrated by the following examples, which should not be construed as limiting in any way.

### Example 1: Synthesis of Probenecid Analogs

Illustrative examples of the general synthesis of probenecid analogs of the present technology are shown in Schemes 1-14.

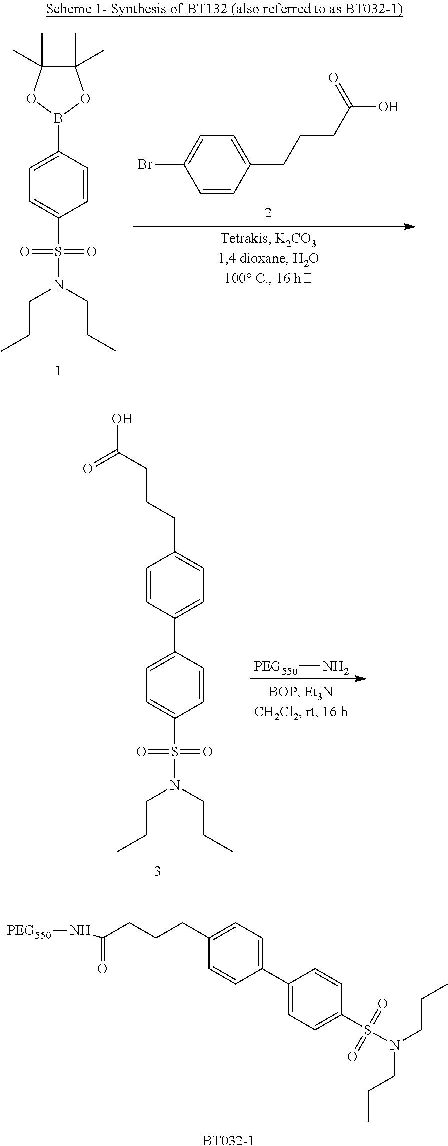

General procedure for Suzuki biaryl coupling reaction. To a solution of dioxaborolane 1 (Scheme 1; 1.5 mmol, 1.0 eq) in aqueous dioxane (5.0 mL) was added 4-(4-bromophenyl)butanoic acid (1.65 mmol, 1.1 eq), potassium phosphate (4.5 mmol, 3.0 eq) and palladium acetate ((Pd(OAc)₂), 0.075 mmol, 0.5 eq). The reaction mixture was stirred at 100° C. for 18 h. The reaction mixture was cooled to room temperature, diluted with water (10.0 mL), acidified with 1 M aqueous HCl, and extracted with ethyl acetate (10.0 mL×2). The organic phase was washed with brine (20.0 mL), dried over sodium sulfate, filtered, and concentrated under reduced pressure to give a residue. The residue was purified by chromatography. 4-(4′-(N,N-dipropylsulfamoyl)-[1,1′-biphenyl]-4-yl)butanoic acid 3, also referred to herein as BT032, (0.8 mmol, 76% yield) was obtained as a white solid. MS: m/z=404 (M+H)⁺.

General procedure for amide coupling reaction and formation of the PEG amides. To a solution of acid 3 (Scheme 1; 1.0 mmol, 1.0 eq) and polyethylene glycol amine ((PEG₅₅₀-NH₂), 1.2 mmol, 1.2 eq) in dichloromethane (3.0 mL) was added BOP (benzotriazol-1-yloxytris(dimethylamino)phosphonium hexafluorophosphate, 1.2 mmol, 1.2 eq) and triethylamine (2.0 mmol, 2 eq). The reaction mixture was stirred at 25° C. for 16 h. The reaction mixture was poured into water and extracted with ethyl acetate (10.0 mL×2). The combined organic layers were washed sequentially with 1 M aqueous HCl (10 mL), saturate aqueous sodium bicarbonate (10 mL), brine (10.0 mL×2), dried over Na₂SO₄, filtered, and concentrated under reduced pressure to give a residue. The residue was purified by combi-flash chromatography eluted with 5-10% MeOH in CH₂Cl₂. PEG₅₅₀ amide BT032-1 (55 mg, 0.26 mmol, 26% yield) was obtained as a white solid. MS (electron spray ionization (ESI)=936 (average MW).

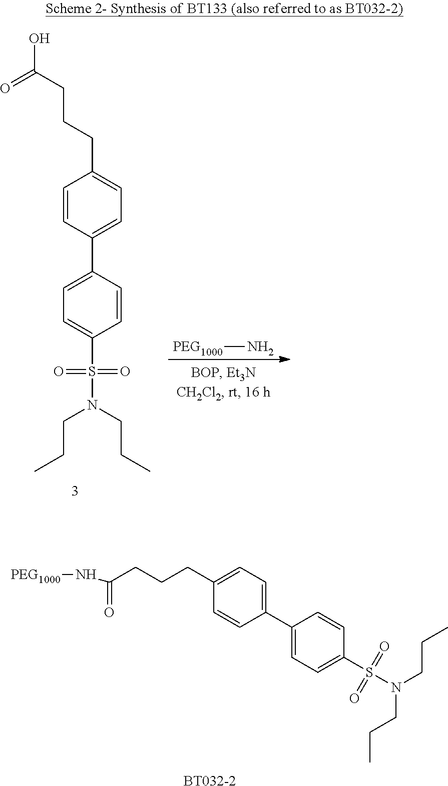

PEG₁₀₀₀ amide BT032-2 was prepared from compound 3 (prepared as in Scheme 1) by adapting the general procedure of Scheme 1 for PEG amidation using NH₂-PEG₁₀₀₀ as indicated in Scheme 2. Reaction details are found in the table below. BT032-2 was obtained as a white solid (100 mg) in 49% yield. MS (ESI)=1386 (average MW).

| S. No. | Reaction | Scale | | Conditions | Yield | Comments |
|---|---|---|---|---|---|---|
| 1 | 3 to 5 | 0.20 | g | Compound 3 (1.0 equiv.), | 0.10 g | Compound 3 consumed; |
| | | | | Compound 4 (1.2 equiv.), | (49%) | TLC showed formation of |
| | | | | Pd(dppf)Cl₂CH₂Cl₂ (0.2 | | new spot; after work-up crude |
| | | | | equiv.), Cul (0.5 equiv.), | | was purified by combi-flash |
| | | | | Et₃N (3 equiv.), | | chromatography eluted with |
| | | | | 1,4-dioxane, 100° C., 4 h | | 30-50% EtOAc in hexanes; |
| | | | | | | ¹H NMR and UPLC |
| | | | | | | consistent |
| 2 | 1 to 3 | 5.0 | g | Compound 1 (1.0 equiv.), | 2.0 g | Compound 1 consumed; |
| | | | | Compound 2 (1.1 equiv.), | (30%) | TLC showed formation of |
| | | | | Pd(OAc)₂ (0.05 equiv.), | | new spot; after work-up crude |
| | | | | K₃PO₄ (3 equiv.), | | was purified by combi-flash |
| | | | | 1,4-dioxane, H₂O, | | chromatography eluted with |
| | | | | 100° C., 16 h | | 30-50% EtOAc in hexanes; |
| | | | | | | ¹H NMR and UPLC |
| | | | | | | consistent |

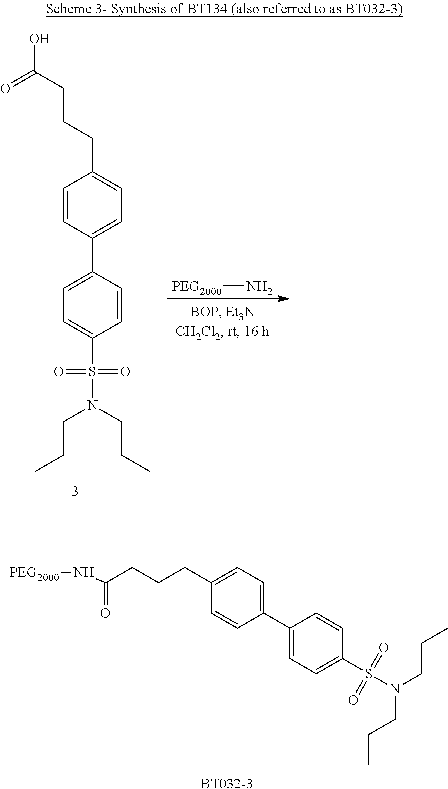

PEG₂₀₀₀ amide BT032-3 was prepared from compound 3 (prepared as in Scheme 1) by adapting the general procedure of Scheme 1 for PEG amidation using NH₂-PEG₂₀₀₀ as indicated in Scheme 3. Reaction details are found in the table below. BT032-3 was obtained as a white solid (800 mg) in 6700 yield. MS (ESI)=2386 (average MW).

| S. No. | Reaction | Scale | Conditions | Yield | Comments |
|---|---|---|---|---|---|
| 1 | 3 to | 200 mg | Compound 3 (1.0 | 800 mg | Compound 3 consumed; TLC |
| | BT032-3 | | equiv.), PEG₍₂₀₀₀₎- | (67%) | showed formation of new spot; after |
| | | | NH₂ (1.2 equiv.), | | work-up crude was purified by |
| | | | BOP (1.2 equiv.), | | combi-flash chromatography eluted |
| | | | Et₃N (2 equiv.), | | with 5-10% MeOH in CH₂Cl₂; ¹H |
| | | | CH₂Cl₂, rt, 16 h | | NMR consistent and HPLC analysis |
| | | | | | in progress. |

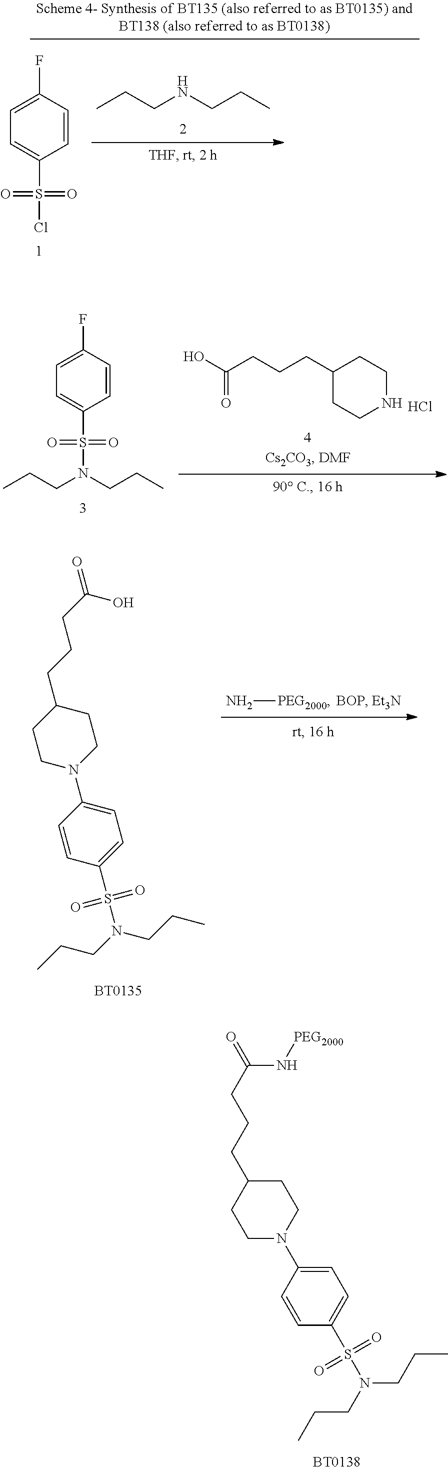

Preparation of BT0135. To a solution of fluoride 3, the preparation of which is described in Kayumov, M., et al., Advanced Synthesis & Catalysis 362(4):776-781 (2020) (Scheme 4; 1.0 mmol, 1.0 eq) and piperidine 4 (1.1 eq.) in dimethylsulfoxide (5 mL) was added excess cesium carbonate (4.0 eq). The reaction mixture was stirred at 90° C. for 16 h. The reaction mixture was poured into water (5 mL), acidified with 1 M aqueous HCl, and extracted with ethyl acetate (10.0 mL×2). The organic phase was washed with brine (20.0 mL), dried over sodium sulfate, filtered, and concentrated under reduced pressure to give a residue. The residue was purified by chromatography. 4-(1-(4-(N,N-dipropylsulfamoyl)-phenyl)piperidin-4-yl)butanoic acid BT0135 (0.53 mmol, 53% yield) was obtained as a white solid. MS: m/z=411 (M+H)⁺.

PEG₂₀₀₀ amide BT0138 was prepared by adapting the general procedure of Scheme 1 for PEG amidation using NH₂-PEG₂₀₀₀ as indicated in Scheme 4. Reaction details are found in the table below. BT0138 was obtained as a white solid (1.40 g) in 48% yield. MS (ESI)=2408 (average MW).

| S. No. | Reaction | Scale | | Conditions | Yield | Comments |
|---|---|---|---|---|---|---|
| 1 | 1 to 3 | 5.0 | g | Compound 1 (1.0 | 6.20 g | Compound 1 consumed; TLC |
| | | | | equiv.), Compound | (92%) | showed formation of new spot; after |
| | | | | 2 (3.2 equiv.), | | work-up crude was used as such for |
| | | | | rt, 2 h | | next reaction; ¹H NMR and LCMS |
| | | | | | | consistent |
| 2 | 3 to | 250 | mg | Compound 3 (1.0 | 140 mg | Compound 3 consumed; TLC |
| | BT0135 | | | equiv.), Compound | (36%) | showed formation of new spot; after |
| | | | | 4 (1.1 equiv.), | | work-up crude was purified by |
| | | | | Cs₂CO₃ (4 equiv.), | | combi-flash chromatography eluted |
| | | | | DMSO, 90° C., 16 h | | with 10-50% EtOAc in hexanes; ¹H |
| | | | | | | NMR and LCMS consistent |
| 3 | 3 to | 2.0 | g | Compound 3 (1.0 | 1.70 g | Compound 3 consumed; TLC |
| | BT0135 | | | equiv.), Compound | (53%) | showed formation of new spot; after |
| | | | | 4 (1.1 equiv.), | | work-up crude was purified by |
| | | | | Cs₂CO₃ (4 equiv.), | | combi-flash chromatography eluted |
| | | | | DMSO, 90° C., 16 h | | with 10-50% EtOAc in hexanes; ¹H |
| | | | | | | NMR and LCMS consistent; HPLC |
| | | | | | | analysis is under progress |
| 4 | BT0135 to | 500 | mg | Compound BT0135 | 1.40 g | Compound 3 consumed; TLC |
| | BT0138 | | | (1.0 equiv.), | (48%) | showed formation of new spot; after |
| | | | | PEG₂₀₀₀-NH₂ (1.2 | | work-up crude was purified by |
| | | | | equiv.), BOP (1.2 | | combi-flash chromatography eluted |
| | | | | equiv.), Et₃N (2 | | with 5-10% MeOH in DCM; ¹H |
| | | | | equiv.), CH₂Cl₂, | | NMR and LCMS consistent; HPLC |
| | | | | rt, 16 h | | analysis is under progress |

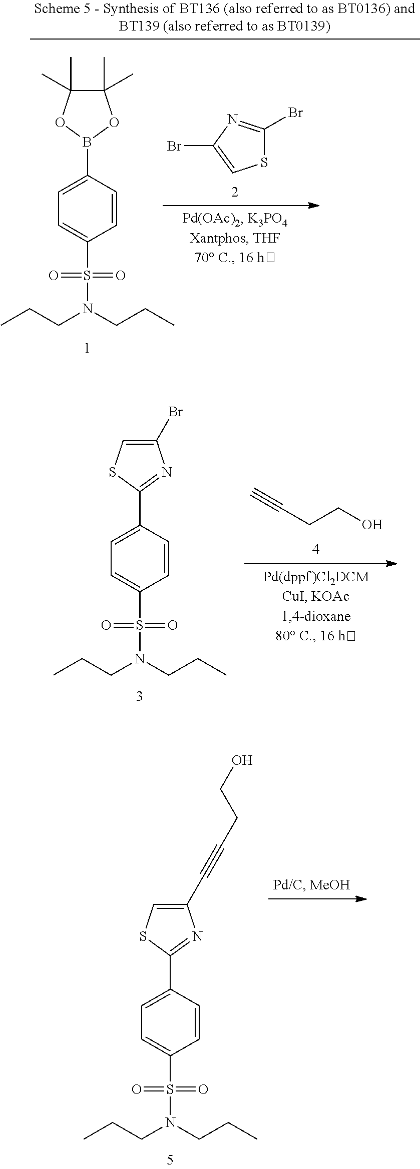

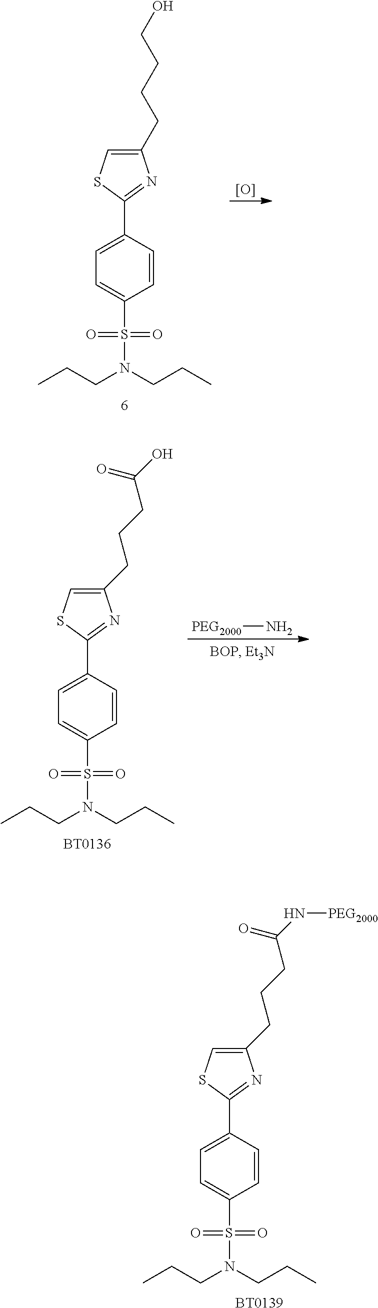

Compound 3 of Scheme 5 was prepared by adapting the general procedure for Suzuki coupling in Scheme 1 using the boronic ester 1 and the dibromothiazole 2. Reaction details are in the table below.

| S. No. | Reaction | Lot | Scale | | Conditions |
|---|---|---|---|---|---|
| 1 | 3 to 5 | IN-PSN-C-34 | 100 | mg | Compound 3 (1.0 equiv.), Compound 4 (1.3 |
| | | | | | equiv.), Pd(dppf)Cl₂, CH₂Cl₂ (0.2 equiv.), CuI |
| | | | | | (0.5 equiv.), Et₃N (3 equiv.), 1,4-dioxane rt to |
| | | | | | 80° C., in sealed tube 16 h |
| 2 | 5 to 6 | IN-PSN-C-34 | 50 | mg | Compound 5 (1.0 equiv.), 10% Pd/C (25 mg), |
| | | | | | H2 bladder, methanol rt 16 h |
| 3 | 1 to 3 | IN-PSN-C-35 | 2.5 | g | Compound 1 (1.0 equiv.), Compound 2 (1.2 |
| | | | | | equiv.), Pd(OAc)₂ (0.025 equiv.), Xantphos |
| | | | | | (0.025 equiv.), K₃PO₄ (3 equiv.), THF, sealed |
| | | | | | tube, rt to 70° C., 16 h |
| 1 | 5 to 6 | IN-PSN-C-52 | 10.0 | g | Compound 5 (1.0 equiv.), 10% Pd/C (25 mg), |
| | | | | | autoclave, H₂ (200 psi), MeOH, rt, 48 h |
| 2 | BT0136 to | IN-PSN-C-60 | 300 | mg | Compound BT0136 (1.0 equiv.), PEG₍₂₀₀₀₎- |
| | BT0139 | | | | NH₂ (1.2 equiv.), BOP (1.2 equiv.), Et₃N (2 |
| | | | | | equiv.), CH₂Cl₂, rt, 16 h |
| 3 | 6 to BT0136 | IN-PSN-C-62 | 7.50 | g | Compound 6 (1.0 equiv.), Jones reagent (0.5 |
| | | | | | mL, 2.67 molar), acetonitrile, 0° C.-5° C., 2 h |

| S. No. | Yield | Comments |
|---|---|---|
| 1 | 60 mg | Compound 3 consumed; TLC showed formation of new spot; |
| | (65%) | after work-up crude was purified by combi-flash chromatography |
| | | eluted with 40-60% EtOAc in hexane; ¹H NMR consistent |
| 2 | 30 mg | Compound 6 consumed; TLC showed formation of new spot; |
| | (60%) | after work-up crude was purified by combi-flash chromatography |
| | | eluted with 40-60% EtOAc in hexane; ¹H NMR consistent |
| 3 | 2.5 g | Compound 1 & 2 consumed; TLC showed formation of new spot; |
| | (60%) | after work-up crude was purified by combi-flash chromatography |
| | | eluted with 20-30% EtOAc in hexanes; ¹H NMR consistent |
| 1 | 7.50 g | Compound 5 consumed; TLC and UPLC showed formation of |
| | (74%) | new spot; after filtration and concentration, crude was washed |
| | | with hexanes; ¹H NMR consistent |
| 2 | 500 mg | BT0136 consumed; TLC and UPLC showed formation of new |
| | (28%) | spot; after work-up crude was purified by reverse phase combi- |
| | | flash chromatography eluted with 40-80% ACN in water; analysis |
| | | is under progress |
| 3 | 2.40 g | Compound 6 consumed; TLC and UPLC showed formation of |
| | (31%) | new spot; after work-up crude was purified by reverse phase |
| | | combi-flash chromatography eluted with 5-10% MeOH in CH₂Cl₂; |
| | | further it was washed with 10% EtOAc in hexanes; NMR, HPLC |
| | | and UPLC consistent |

Preparation of BT0136. To an ice-cold solution of alcohol 6 (Scheme 5; 1.0 mmol, 1.0 eq) in acetonitrile (1.5 mL) was added an excess of freshly prepared Jones reagent (2.67 molar; 0.5 mL). The reaction mixture was stirred at 0-5° C. for 2 h. The reaction mixture was poured into ice water (5 mL) and extracted with ethyl acetate (10.0 mL×2). The combined organic layers were washed with brine (10.0 mL×2), dried over Na₂SO₄, filtered, and concentrated under reduced pressure to give a residue. The residue was purified by chromatography. 4-(2-(4-(N,N-dipropylsulfamoyl)phenyl)thiazol-4-yl)butanoic acid BT0136 (0.3 mmol, 31% yield) was obtained as a white solid. MS: m/z=411 (M+H)⁺.

PEG₂₀₀₀ amide BT0139 was prepared by adapting the general procedure of Scheme 1 for PEG amidation using NH₂-PEG₂₀₀₀ as indicated in Scheme 5. Reaction details are found in the table below. BT0139 was obtained as a white solid (160 mg) in 28% yield. MS (ESI)=2408 (average MW).

| S. No. | Reaction | Scale | | Conditions | Yield | Comments |
|---|---|---|---|---|---|---|
| 1 | BT0136 to | 300 | mg | Compound BT0136 (1.0 | 500 mg | BT0136 consumed; TLC and UPLC |
| | BT0139 | | | equiv.), PEG₍₂₀₀₀₎- | (28%) | showed formation of new spot; after |
| | | | | NH₂ (1.2 equiv.), | | work-up crude was purified by |
| | | | | BOP (1.2 equiv.), | | reverse phase combi-flash |
| | | | | Et₃N (2 equiv.), | | chromatography eluted with 40-80% |
| | | | | CH₂Cl₂, rt, 16 h | | ACN in water; ¹H NMR, HPLC and |
| | | | | | | UPLC consistent |
| 2 | 6 to | 7.50 | g | Compound 6 (1.0 | 2.40 g | Compound 6 consumed; TLC and |
| | BT0136 | | | equiv.), Jones | (31%) | UPLC showed formation of new |
| | | | | reagent (0.5 mL, | | spot; after work-up crude was |
| | | | | 2.67 molar), | | purified by combi-flash |
| | | | | acetonitrile, | | chromatography eluted with 5-10% |
| | | | | 0° C.-5° C., 2 h | | MeOH in CH₂Cl₂; further it was |
| | | | | | | washed with 10% EtOAc in hexanes; |
| | | | | | | ¹H NMR, HPLC and UPLC |
| | | | | | | consistent |

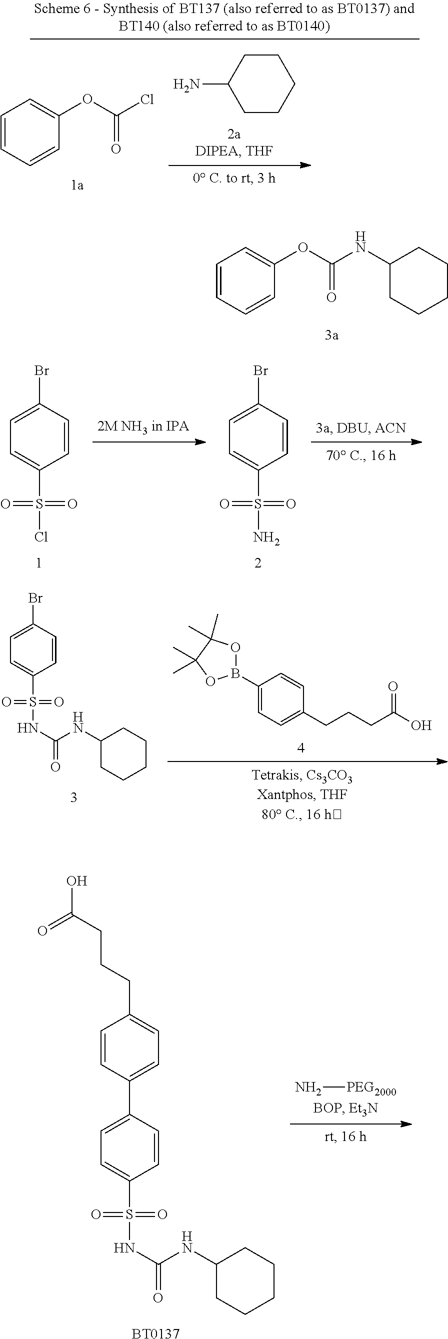

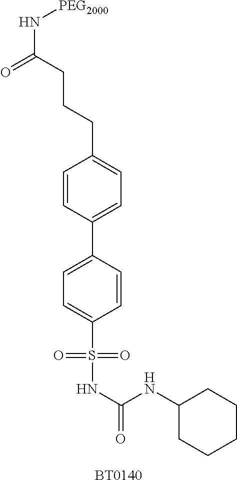

Acid BT137 was prepared by adapting the general procedure of Scheme 1 for Suzuki coupling using the aryl bromide and boronic acid ester indicated in Scheme 6. Reaction details are found in the table below. BT0137 was obtained as a white solid in 89% yield. MS: m/z=445 (M+H)⁺.

PEG₂₀₀₀ amide BT0140 was prepared by adapting the general procedure of Scheme 1 for PEG amidation using NH₂-PEG₂₀₀₀ as indicated in Scheme 6. Reaction details are found in the table below. BT0140 was obtained as a white solid (160 mg) in 33% yield. MS (ESI)=2442 (average MW).

| S. No. | Reaction | Scale | | Conditions | Yield | Comments |
|---|---|---|---|---|---|---|
| 1 | 1a to 3a | 5.0 | g | Compound 1a (1.0 | 6.20 g | Compound 1 consumed; TLC |
| | | | | equiv.), Compound | (89%) | showed formation of new spot; after |
| | | | | 2a (1. lequiv,), | | work-up crude was washed with |
| | | | | DIPEA (1.2 equiv.), | | hexanes and dried, ¹H NMR and |
| | | | | THF, 0° C. to | | LCMS consistent |
| | | | | RT, 3 h | | |
| 2 | 2 to 3 | 500 | mg | Compound 2 (1.0 | 600 mg | Compound 2 consumed; TLC |
| | | | | equiv.), Compound | (79%) | showed formation of new spot; after |
| | | | | 3a (1.2 equiv.), | | work-up crude was purified by |
| | | | | DBU (1.5 equiv.), | | combi-flash chromatography eluted |
| | | | | ACN, 70° C., 16 h | | with 30-60% ethyl acetate in hexanes; |
| | | | | | | ¹H NMR and LCMS consistent |
| 3 | 3 to | 500 | mg | Compound 3 (1.0 | 160 mg | Compound 3 consumed; TLC |
| | BT0137 | | | equiv.), Compound | (89%) | showed formation of new spot; after |
| | | | | 4 (1.1 equiv.), | | work-up crude was purified by |
| | | | | tetrakis (0.1 equiv.), | | combi-flash chromatography eluted |
| | | | | K₂CO₃ (1.2 equiv.), | | with 5-10% MeOH in CH₂Cl₂; further |
| | | | | 1,4-dioxane, 100° C., | | it was purified by reverse phase |
| | | | | 16 h | | combi-flash chromatography eluted |
| | | | | | | with 40-80% ACN in water; ¹H NMR |
| | | | | | | consistent |

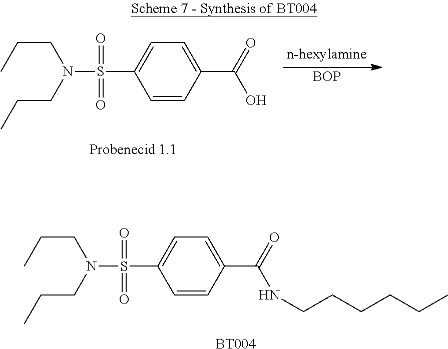

To a solution of acid probenecid 1.1 (Cayman Chemical Company, 1180 East Ellsworth Rd., Ann Arbor, MI, 48108; 1 mmol, 1.0 eq) and n-hexylamine (1.2 eq) in dichloromethane (3.0 mL) was added benzotriazol-1-yloxytris(dimethylamino)phosphonium hexafluorophosphate (BOP, 1.2 eq) and diisopropylethylamine (2.0 mmol, 2 eq). The reaction mixture was stirred at 25° C. for 16 h. The reaction mixture was poured into water and extracted with ethyl acetate (10 mL×2). The combined organic layers were washed sequentially with 1 M aqueous hydrochloric acid (HCl; 10 mL), saturated aqueous sodium bicarbonate (NaHCO₃; 10 mL), brine (10 mL×2), dried over sodium sulfate (sodium sulfate), filtered, and concentrated under reduced pressure to give a residue. The residue was purified by chromatography. 4-(N,N-Dipropylsulfamoyl)-N-hexylbenzamide BT004 (0.89 mmol, 89% yield) was obtained as a white solid. MS: m/z=370 (M+H)⁺.

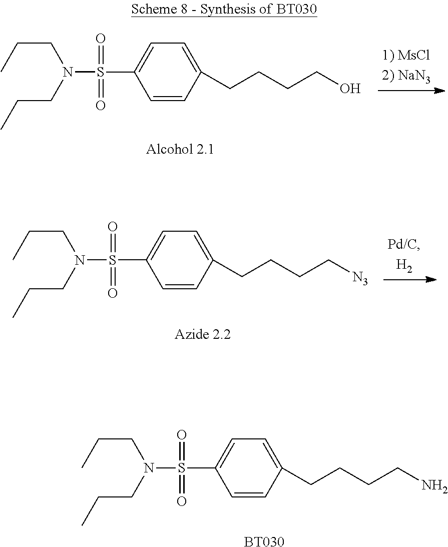

To a cold solution of alcohol 2.1 (Intonation Research Laboratories, A-1B, Chilka Nagar Main Rd, Industrial Development Area, Nacharam, Secunderabad, Telangana 500076, India; 1 mmol, 1.0 eq) in dichloromethane (DCM; 5 mL) was added and triethylamine (2 eg) followed by methane sulfonyl chloride (MsCl, 1.2 eq). The reaction mixture was stirred at 5° C. for 3 h. The reaction mixture was poured into water and extracted with ethyl acetate (10 mL×2). The combined organic layers were washed sequentially with 1 M aqueous hydrochloric acid (HCl; 10 mL), saturated aqueous sodium bicarbonate (NaHCO₃; 10 mL), brine (10 mL×2), dried over sodium sulfate (sodium sulfate), filtered, and concentrated under reduced pressure to give the corresponding methane sulfonate. The crude sulfonate was dissolved in dimethylformamide (5 mL) and the solution chilled. Sodium azide (NaN₃; 2 eq) was then added and the reaction mixture was stirred at 60° C. for 6 h. The reaction mixture was poured into water and extracted with ethyl acetate (10 mL×2). The combined organic layers were washed sequentially with 1 M aqueous hydrochloric acid (HCl; 10 mL), saturated aqueous sodium bicarbonate (NaHCO₃; 10 mL), brine (10 mL×2), dried over sodium sulfate (sodium sulfate), filtered, and concentrated under reduced pressure to give the corresponding azide 2.2. Compound 2.2 was used directly in the next reaction. Azide 2.2 was dissolved in ethanol (10 mL) and 10% palladium on carbon (Pd/C; 0.3 eq). The reaction mixture was stirred under an atmosphere of hydrogen gas at ambient temperature and pressure for 12 h. The reaction mixture was filtered to remove the Pd/C catalyst, the solvent was removed under reduced pressure to give a residue which was purified by chromatography. 4-(4-Aminobutyl)-N,N-dipropylbenzenesulfonamide BT030 (0.45 mmol, 45% yield) was obtained as a white solid. MS: m/z=389 (M−H).

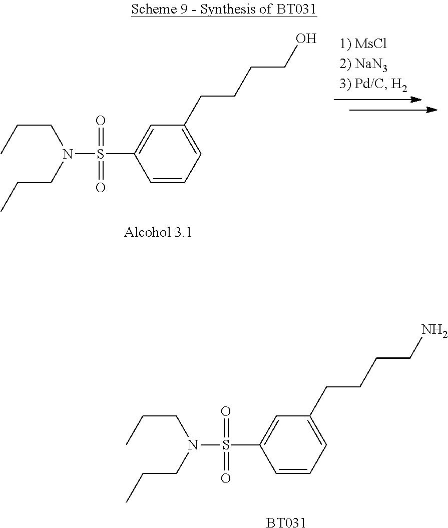

The same reaction conditions used to prepare BT030 were applied to the synthesis of BT031 starting with alcohol 3.1. 3-(4-Aminobutyl)-N,N-dipropylbenzenesulfonamide BT030 was obtained as a white solid. MS: m/z=389 (M−H).

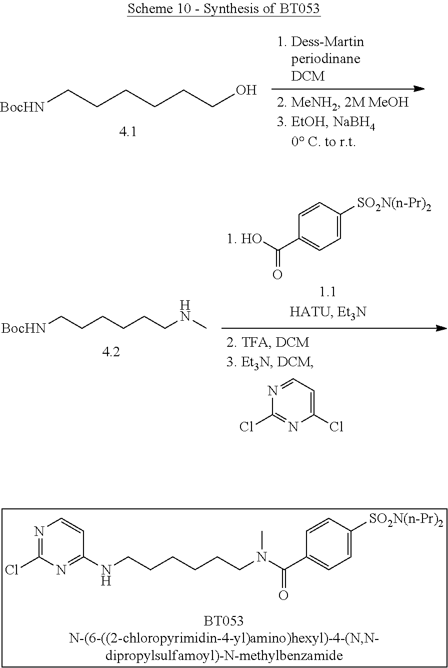

tert-Butyl (6-(methylamino)hexyl)carbamate (4.2). To a solution of tert-butyl (6-hydroxyhexyl)carbamate 4.1 (4.0 g, 18.4 mmol) in DCM (100 mL) at 0° C. was added the periodinane (9.37 g, 22.0 mmol) and the resulting mixture was stirred for 2 h. The reaction was then quenched with a saturated solution of sodium bisulfite (100 mL) and a saturated solution of NaHCO₃ (100 mL). The phases were separated, and the aqueous phase was extracted with DCM (100 mL). Organic phases were combined and dried over MgSO₄, filtered and concentrated under reduced pressure to afford the crude aldehyde. Freshly prepared aldehyde in EtOH (80 mL) was stirred at room temperature. After 2 min, the MeNH2 2M in MeOH (73 mL) was added, and the mixture vigorously stirred. After stirring for 3 h, the resulting mixture was cooled down to 0° C. and NaBH₄ (731 mg, 19.3 mmol) was added. The reaction was stirred for 30 min and the resulting mixture was quenched with a saturated solution of NaHCO₃ and the organic material was extracted with DCM (3×300 mL), dried over sodium sulfate and concentrated under reduced pressure affording crude 4.2 (4.2 g, 99%). LC-MS: RT=1.09 min; MS cal.: 230.35; Mass found: [M-Boc+H]: 175.0. ¹H NMR (500 MHz, CDCl₃) δ 4.51 (s, 1H), 3.10 (d, J=6.0 Hz, 2H), 2.64-2.52 (m, 2H), 2.43 (s, 3H), 1.54-1.46 (m, 4H), 1.44 (s, 9H), 1.33 (dd, J=6.7, 2.9 Hz, 4H).

N-(6-((2-Chloropyrimidin-4-yl)amino)hexyl)-4-(N,N-dipropylsulfamoyl)-N-methylbenzamide (BT053). Triethylamine (10.3 mL, 73.6 mmol) was added to a solution of 4-(N,N-dipropylsulfamoyl)benzoic acid 1.1 (5.25 g, 18.4 mmol) dissolved in DMF (46.0 mL). Then HATU (7.07 g, 18.4 mmol) was added in the mixture at 0° C. and stirred for 5 min. A solution of tert-butyl (6-(methylamino)hexyl)carbamate 4.2 (4.24 g, 18.4 mmol) was added dropwise over 15 min to the mixture at 0° C. and the reaction was allowed to slowly warm up to room temperature and stirred for 18 h. Then EtOAc (50 mL) and water (50 mL) were added, the organic layer was washed with water (2×50 mL), washed with a saturated solution of NaHCO₃ (1×50 mL), washed with brine, dried over anhydrous sodium sulfate and concentrated under reduced pressure affording crude Boc protected amine (8.0 g, 93%) and was used without purification for the next step. LC-MS: RT=1.86 min; MS cal.: 497.69; Mass found: [M-Boc+H]: 398.3. Trifluoroacetic acid (11.4 mL, 148 mmol) was added to a solution of crude dissolved in DCM (36.9 mL). The reaction was stirred for 5 h at room temperature. The reaction was followed by LC-MS and starting material was still present. Trifluoroacetic acid (2.0 mL) was added and the reaction stirred for 18 h. The mixture was washed with a saturated solution of NaHCO₃ (2×20 mL), dried over anhydrous sodium sulfate and concentrated under reduced pressure affording the crude amine (7.65 g) and was used without purification for the next step. LC-MS: RT=1.34 min; MS cal.: 397.58; Mass found: [M+H]: 398.3. 2,4-Dichloropyrimidine (3.64 g, 23.9 mmol) was added to a solution of crude and triethylamine (20.6 mL, 147 mmol) dissolved in DCM (46.0 mL). The reaction was stirred for 3 h at 0° C. (the reaction was followed by LCMS and wasn't yet completed), Et3N (4.0 mL) was added and the resulting mixture was stirred for 18 h at room temperature. Then water (40 mL) was added and extracted with EtOAc (2×40 mL), washed with brine, dried over anhydrous sodium sulfate and concentrated under reduced pressure. The crude was purified by flash column chromatography (220 g silica, EtOAc in hexanes, 15 to 100%) affording compound BT053 (5.09 g, 54%) as a yellow oil. LC-MS: RT=1.73 min; Purity: 98.9%; MS cal.: 510.09; Mass found: [M+H]: 510.3. ¹H NMR (500 MHz, CDCl3) δ 7.99 (s, 1H), 7.85 (d, J=8.1 Hz, 2H), 7.50 (d, J=7.9 Hz, 2H), 6.26 (d, J=6.0 Hz, 1H), 5.31 (s, 1H), 3.56 (t, J=6.9 Hz, 1H), 3.22-3.13 (m, 2H), 3.09 (s, 6H), 2.96 (s, 3H), 2.89 (d, J=11.7 Hz, 2H), 1.84-1.46 (m, 12H), 0.88 (t, J=7.4 Hz, 6H).

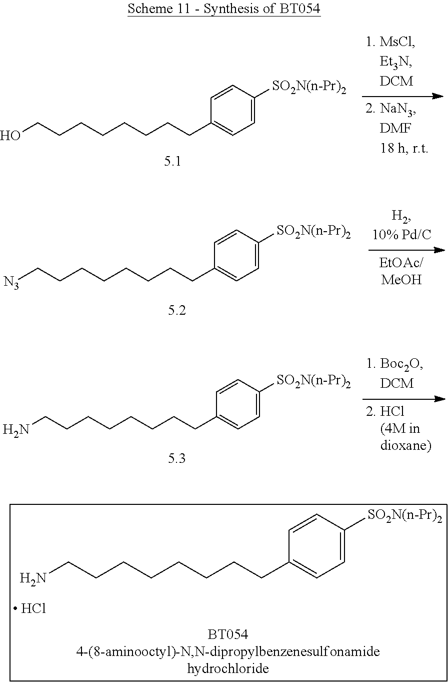

4-(8-Azidooctyl)-N,N-dipropylbenzenesulfonamide (5.2). Methanesulfonyl chloride (1.53 mL, 19.6 mmol) was added to a solution of 4-(8-hydroxyoctyl)-N,N-dipropylbenzenesulfonamide 5.1 (4.84 g, 13.1 mmol) and triethylamine (3.67 mL, 26.2 mmol) dissolved in DCM (43.7 mL) at 0° C. The reaction was stirred for 30 min at 0° C. and 30 min at room temperature. Then water (30 mL) was added and extracted with EtOAc (2×30 mL), washed with brine, dried with anhydrous sodium sulfate and concentrated under reduced pressure affording the crude mesylate as a yellow oil. LC-MS: RT=2.08 min; MS cal.: 447.65; Mass found: [M+H]: 448.4. ¹H NMR (400 MHz, CDCl3) δ 7.71 (d, J=8.4 Hz, 2H), 7.48 (d, J=8.4 Hz, 2H), 4.24 (t, J=6.5 Hz, 2H), 3.12-3.03 (m, 4H), 3.00 (s, 3H), 2.44 (t, J=7.0 Hz, 2H), 1.84-1.75 (m, 2H), 1.69-1.59 (m, 2H), 1.55-1.43 (m, 8H), 0.86 (t, J=7.4 Hz, 6H). Sodium azide (1.71 mL, 26.2 mmol) was added to a solution of crude mesylate (13.1 mmol) dissolved in DMF (65.5 mL). The reaction was stirred for 18 h at room temperature. Then EtOAc (30 mL) and water (30 mL) were added, the organic phase was washed with water (3×30 mL), washed with brine, dried over anhydrous sodium sulfate and concentrated under reduced pressure. The crude was purified by flash column chromatography (100 g silica, 1 to 20% EtOAc/Hexanes) affording azide 5.2 (3.65 g, 71% over 2 steps) as a colourless oil. LC-MS: RT=2.32 min; MS cal.: 394.67; Mass found: [M−N2]: 367.4. ¹H NMR (400 MHz, CDCl3) δ 7.70 (d, J=8.3 Hz, 2H), 7.28 (d, J=8.3 Hz, 2H), 3.25 (t, J=6.9 Hz, 2H), 3.10-3.00 (m, 4H), 2.69-2.61 (m, 2H), 1.70-1.45 (m, 8H), 1.42-1.28 (m, 8H), 0.87 (t, J=7.4 Hz, 6H).

4-(8-Aminooctyl)-N,N-dipropylbenzenesulfonamide (5.3). Palladium, 10% weight on activated carbon (150 mg) was added to a solution of 4-(8-azidooctyl)-N,N-dipropylbenzenesulfonamide 5.2 (1.45 g, 3.67 mmol) dissolved in EtOAc (36.9 mL) and MeOH (3.69 mL). After flushing (×3) the resulting mixture and adding hydrogen with a balloon, the reaction was stirred for 2 h at room temperature under hydrogen atmosphere. The mixture was filtered over Celite (MeOH was used for the washing) and concentrated under reduced pressure affording crude amine 5.3 (1.23 g, 91%) as a yellow oil. LC-MS: RT=1.57 min; MS cal.: 368.58; Mass found: [M+H]: 369.3.

4-(8-Aminooctyl)-N,N-dipropylbenzenesulfonamide hydrochloride (BT054 HCl salt). Di-tert-butyldicarbonate (1.53 mL, 6.67 mmol) was added to a solution of crude 5.3 (1.23 g, 3.34 mmol) dissolved in DCM (16.7 mL). The reaction was stirred for 30 min at room temperature. The resulting mixture was concentrated under reduced pressure and purified by flash column chromatography (100 g silica, 5 to 35% EtOAc/Hexanes) affording the Boc protected amine (1.45 g, 93%) as a yellow oil. LC-MS: RT=2.24 min; MS cal.: 468.69; Mass found: [M-Boc+H]: 369.4. Hydrochloric acid (3.09 mL, 12.4 mmol) (4 M solution in dioxane) was added to a solution of the Boc protected amine (1.45 g, 3.09 mmol). The reaction was stirred for 1 h at room temperature. Air was flushed in the flask to concentrate the resulting mixture affording BT054 HCl salt (1.20 g, 96%) as a white solid. LC-MS: RT=1.64 min; Purity: 95.5%; MS cal.: 405.04; Mass found: [M-Cl]: 369.2. ¹H NMR (500 MHz, CDCl3) δ 8.28 (s, 3H), 7.69 (d, J=8.2 Hz, 2H), 7.27 (d, J=7.6 Hz, 2H), 3.08-3.03 (m, 4H), 2.95 (m, 2H), 2.64 (t, J=7.7 Hz, 2H), 1.80-1.71 (m, 2H), 1.64-1.59 (m, 2H), 1.54 (dq, J=14.9, 7.5 Hz, 4H), 1.42-1.35 (m, 2H), 1.30 (s, 6H), 0.86 (t, J=7.4 Hz, 6H).

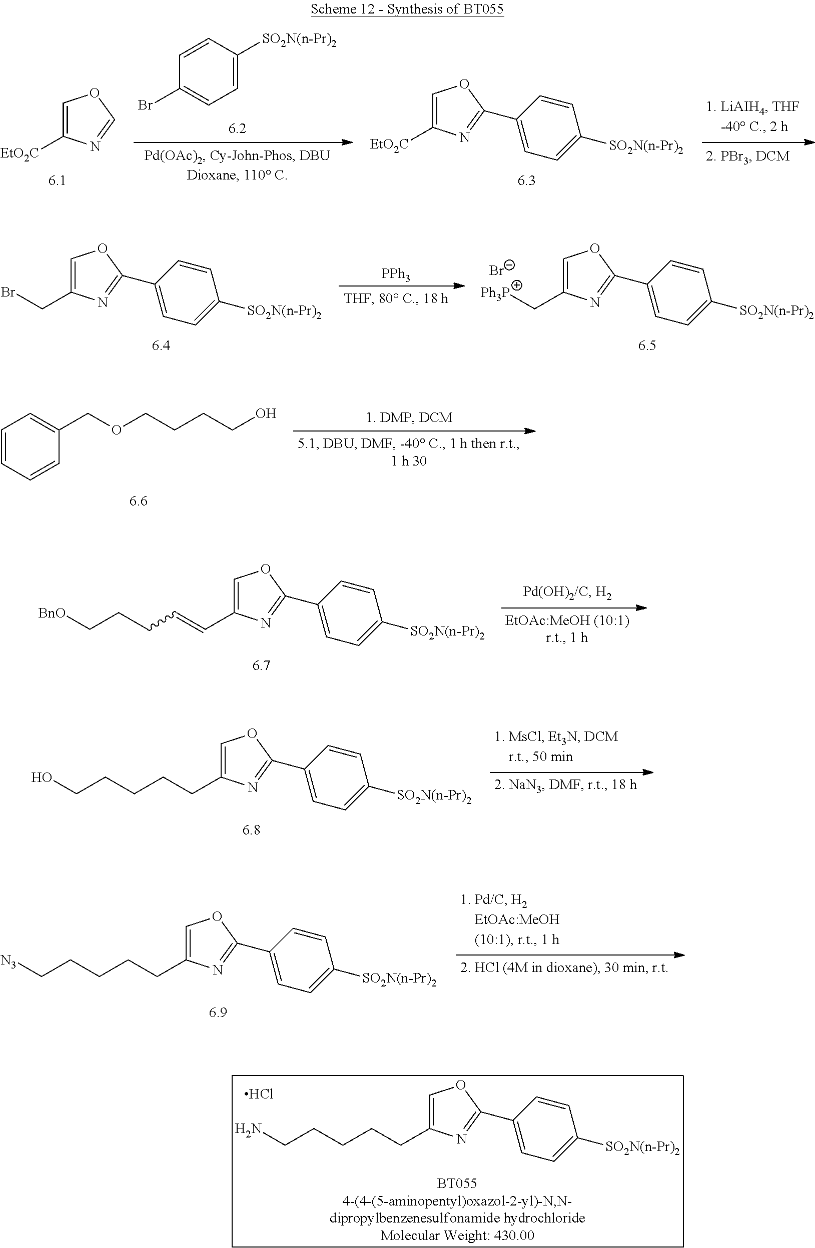

Ethyl 2-(4-(N,N-dipropylsulfamoyl)phenyl)oxazole-4-carboxylate (6.3). Ethyl oxazole-4-carboxylate 6.1 (4.0 g, 28 mmol) was placed in a sealed tube (250 mL) with DBU (8.47 mL, 57 mmol), Pd(OAc)₂ (318.2 mg, 1.4 mmol) and Cy-John-Phos ligand (994 mg, 2.8 mmol). A solution of bromide 6.2 (9.0 g, 28 mmol) in dry dioxane (80 mL) was added and the resulting mixture was purged with nitrogen for a period of 10 min. The mixture was stirred at 110° C. for 18 h. After filtration through Celite and concentration in vacuo, the crude product was purified by flash column chromatography (330 g silica, 5 to 35% EtOAc/Hexanes) affording 6.3 (6.70 g, 62%) as a white powder. LC-MS: RT=1.86 min; MS cal.: 380.46; Mass found: [M+H]: 381.2. ¹H NMR (400 MHz, CDCl3) δ 8.32 (s, 1H), 8.25-8.22 (m, 2H), 7.93-7.88 (m, 2H), 4.44 (q, J=7.1 Hz, 2H), 3.14-3.07 (m, 4H), 1.57-1.49 (m, 4H), 1.42 (t, J=7.1 Hz, 3H), 0.87 (t, J=7.4 Hz, 6H).

4-(4-(Bromomethyl)oxazol-2-yl)-N,N-dipropylbenzenesulfonamide (6.4). Lithium aluminum hydride (10.1 mL, 20.1 mmol, 2.0 M in THF) was added dropwise over 10 min to a solution of ethyl 2-(4-(N,N-dipropylsulfamoyl)phenyl)oxazole-4-carboxylate 6.3 (3.83 g, 10.1 mmol) dissolved in THE (101 mL) at −40° C. (using a bath of dry ice and acetonitrile) under argon atmosphere. The reaction was stirred for 2 h at −40° C. Then a solution of sat. Rochelle salt (30 mL) was added in the reaction mixture at −40° C., anhydrous sodium sulfate was added and the mixture was vigorously agitated at room temperature. The mixture was filtered and concentrated under reduced pressure affording crude alcohol (3.17 g, 93%) as a yellow solid. LC-MS: RT=1.56 min; MS cal.: 338.42; Mass found: [M+H]: 339.3. Phosphorus tribromide (1.78 mL, 18.7 mmol) was added dropwise to a solution of crude alcohol (3.17 g, 9.37 mmol) dissolved in DCM (62.4 mL) at 0° C. The reaction was stirred at this temperature for 1 h. The mixture was stirred at room temperature for 4 h. Then water (50 mL) was added and extracted with EtOAc (2×50 mL), washed with brine, dried over anhydrous sodium sulfate, filtered and concentrated under reduced pressure. The crude was purified with a silica pad (30% EtOAc/Hexanes) affording 6.4 (2.68 g, 71%) as a yellow powder. LC-MS: RT=1.91 min; MS cal.: 401.32; Mass found: [M+H]: 403.0.

((2-(4-(N,N-dipropylsulfamoyl)phenyl)oxazol-4-yl)methyl)triphenylphosphonium bromide (6.5). Triphenyl-phosphine (1.47 g, 5.61 mmol) was added to a solution of 6.4 (2.25 g, 5.61 mmol) dissolved in THE (22.4 mL). The reaction was refluxed for 18 h. Then the resulting mixture was concentrated under reduced pressure affording crude 6.5 (3.73 g, 100%) as a pale yellow solid. The material was used in the next step without purification. LC-MS: RT=1.80 min; MS cal.: 663.60; Mass found: [M-Br]: 583.4. ¹H NMR (500 MHz, CDCl3) δ 8.48 (d, J=4.4 Hz, 1H), 7.90 (ddt, J=9.4, 3.6, 1.3 Hz, 8H), 7.84-7.76 (m, 5H), 7.69-7.64 (m, 6H), 5.60 (d, J=14.0 Hz, 2H), 3.13-3.04 (m, 4H), 1.60-1.50 (m, 4H), 0.87 (t, J=7.4 Hz, 6H).

4-(4-(5-(Benzyloxy)pent-1-en-1-yl)oxazol-2-yl)-N,N-dipropylbenzenesulfonamide (6.7). Dess-martin periodinane (2.91 g, 6.73 mmol) was added to a solution of 4-(benzyloxy)butan-1-ol 6.6 (987 μL, 5.61 mmol) dissolved in DCM (28.1 mL) at 0° C. The reaction was stirred at room temperature for 1 h 30 min. Then a sat. solution of NaHCO₃ (50 mL) and a sat. solution of Na₂S₂O₃ (50 mL) were added and the resulting mixture was stirred for 30 min, extracted with DCM (2×50 mL), washed with brine, dried over anhydrous sodium sulfate and concentrated under reduced pressure affording crude aldehyde as a yellow oil. The crude was used in the next step reaction without purification. 1,8-diazabicyclo[5.4.0]undec-7-ene (DBU) (1.28 mL, 8.42 mmol) was added to a solution of crude aldehyde and phosphonium salt 6.5 (3.72 g, 5.61 mmol) dissolved in DMF (28.1 mL) at −40° C. under argon. The reaction was stirred at this temperature for 1 h then at r.t. for 1 h 30 min. Then water (50 mL) and EtOAc (50 mL) were added, washed with water (2×50 mL), washed with brine (×2), dried over anhydrous sodium sulfate and concentrated under reduced pressure. The crude was purified by flash column chromatography (silica 100 g, 10 to 100% EtOAc/hexanes) affording 6.7 (1.78 g, 66%) as a yellow oil. LC-MS: RT=2.25 min; MS cal.: 482.63; Mass found: [M+H]: 483.4. ¹H NMR (500 MHz, CDCl3) δ 8.20-8.13 (m, 2H), 7.91-7.83 (m, 2H), 7.74 (s) and 7.57 (s) (1H, E/Z isomers), 7.37-7.31 (m, 5H), 6.56 (dt, J=15.6, 7.0 Hz, 0.5H), 6.27 (ddd, J=13.5, 5.3, 3.9 Hz, 1H), 5.83 (dt, J=11.5, 7.4 Hz, 0.5H), 4.52 (s, 2H), 3.55 (dt, J=11.4, 6.4 Hz, 2H), 3.14-3.08 (m, 4H), 2.60 (qd, J=7.5, 1.6 Hz, 1H), 2.34 (q, J=6.9 Hz, 1H), 1.89-1.77 (m, 2H), 1.60-1.50 (m, 4H), 0.87 (td, J=7.4, 2.8 Hz, 6H).

4-(4-(5-Hydroxypentyl)oxazol-2-yl)-N,N-dipropylbenzenesulfonamide (6.8). Palladium hydroxide (20 wt. % Pd on carbon wet, 178 mg) was added to a solution of 6.7 (1.78 g, 3.69 mmol) dissolved in MeOH (3.35 mL) and EtOAc (33.5 mL). After flushing (×3) the resulting mixture and adding hydrogen with a balloon, the reaction was stirred for 1 h at room temperature under hydrogen atmosphere. The mixture was filtered over Celite (EtOAc was used for the washing) and concentrated under reduced pressure affording crude alcohol 6.8 (1.11 g, 76%) as a white solid. LC-MS: RT=1.75 min; MS cal.: 394.53; Mass found: [M+H]: 395.3. ¹H NMR (500 MHz, CDCl3) δ 8.12 (d, J=8.5 Hz, 2H), 7.86 (d, J=8.5 Hz, 2H), 7.47 (s, 1H), 3.65 (t, J=6.5 Hz, 2H), 3.14-3.03 (m, 4H), 2.60 (t, J=7.5 Hz, 2H), 1.72 (dt, J=15.4, 7.7 Hz, 2H), 1.66-1.58 (m, 2H), 1.53 (m, 4H), 1.50-1.42 (m, 2H), 0.86 (t, J=7.4 Hz, 6H).

4-(4-(5-Azidopentyl)oxazol-2-yl)-N,N-dipropylbenzenesulfonamide (6.9). Methanesulfonyl chloride (885 μL, 11.4 mmol) was added to a solution of 6.8 (3.00 g, 7.60 mmol) and triethylamine (2.13 mL, 15.2 mmol) dissolved in DCM (38.0 mL) at 0° C. The reaction was stirred at this temperature for 10 min and at r.t. for 50 min. Then water (50 mL) was added, extracted with EtOAc (2×50 mL), washed with brine, dried over anhydrous sodium sulfate and concentrated under reduced pressure affording crude mesylate as a yellow oil. The crude was used in the next step without purification. LC-MS: RT=1.90 min; MS cal.: 472.62; Mass found: [M+H]: 473.3. Sodium azide (993 μL, 15.2 mmol) was added to a solution of crude mesylate (7.60 mmol) dissolved in DMF (38.0 mL). The reaction was stirred for 18 h at room temperature. Then EtOAc (50 mL) and water (50 mL) were added, washed with water (3×50 mL), washed with brine, dried over anhydrous sodium sulfate and concentrated under reduced pressure. The crude was purified by flash column chromatography (silica 100 g, 5 to 100% EtOAc/hexanes) affording 6.9 (2.61 g, 82% over 2 steps) as a colourless oil. LC-MS: RT=2.12 min; MS cal.: 419.54; Mass found: [M+H]: 420.3. ¹H NMR (500 MHz, CDCl3) δ 8.14 (d, J=8.6 Hz, 2H), 7.88 (d, J=8.6 Hz, 2H), 7.49 (s, 1H), 3.29 (t, J=6.9 Hz, 2H), 3.14-3.06 (m, 4H), 2.62 (t, J=7.3 Hz, 2H), 1.78-1.44 (m, 10H), 0.87 (t, J=7.4 Hz, 6H).

4-(4-(5-Aminopentyl)oxazol-2-yl)-N,N-dipropylbenzenesulfonamide hydrochloride (BT055 HCl salt). Palladium (10% wt on activated carbon, 261 mg) was added to a solution of 6.9 (2.61 g, 6.22 mmol) dissolved in MeOH (5.66 mL) and EtOAc (56.6 mL). After flushing (×3) the resulting mixture and adding hydrogen with a balloon, the reaction was stirred for 1 h at room temperature under hydrogen atmosphere. The mixture was filtered over Celite (EtOAc was used for the washing) and concentrated under reduced pressure affording crude amine. Then a solution of hydrochloric acid (4 M in dioxane, 3 eq.) was added and the reaction was stirred for 30 min at r.t. The resulting mixture was concentrated under reduced pressure, agitated in MTBE and filtered affording BT055 HCl salt (2.33 g, 87%) as a white solid. LC-MS: RT=1.48 min; Purity: 97.02%; MS cal.: 430.00; Mass found: [M-Cl]: 394.3. ¹H NMR (500 MHz, CDCl3) δ 8.35 (s, 3H), 8.28 (d, J=8.5 Hz, 2H), 7.92 (d, J=8.6 Hz, 2H), 7.65 (s, 1H), 3.10 (dd, J=8.6, 6.7 Hz, 4H), 3.01 (m, 2H), 2.70 (t, J=7.5 Hz, 2H), 1.81 (m, 4H), 1.54 (m, 6H), 0.86 (t, J=7.4 Hz, 6H).

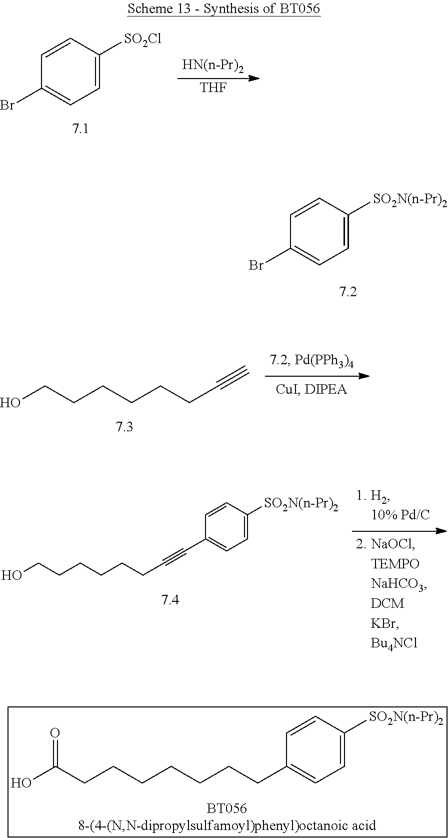

4-Bromo-N,N-dipropylbenzenesulfonamide (7.2). Dipropylamine (31.9 mL, 230 mmol) was added to a solution of 4-bromobenzenesulfonyl chloride 7.1 (20.0 g, 76.7 mmol) dissolved in THE (153 mL) at 0° C. The reaction was stirred for 1 h at room temperature. Then a saturated solution of NH₄Cl (50 mL) was added, extracted with EtOAc (2×50 mL), washed with brine, dried over sodium sulfate and concentrated under reduced pressure affording compound 7.2 (24.6 g, 100% yield) as a white solid. LC-MS: RT=1.95 min; MS cal.: 320.25; Mass found: [M+H]: 320.1. ¹H NMR (400 MHz, CDCl3) δ 7.70-7.59 (m, 4H), 3.10-3.03 (m, 4H), 1.60-1.50 (m, 4H), 0.87 (t, J=7.4 Hz, 6H).

4-(8-Hydroxyoct-1-yn-1-yl)-N,N-dipropylbenzenesulfonamide (7.4). Tetrakis (triphenylphosphine)palladium(0) (5.17 g, 4.39 mmol) was added to a solution of oct-7-yn-1-ol 7.3 (4.52 g, 35.1 mmol), 4-bromo-N,N-dipropylbenzenesulfonamide 7.2 (5.62 g, 17.6 mmol) and CuI (3.34 g, 17.6 mmol) dissolved in diisopropylethylamine (88.0 mL). The reaction was stirred for 2 h at 150° C. Then EtOAc (50 mL) and a saturated solution of NH4Cl (50 mL) were added, extracted with EtOAc (2×50 mL), washed with brine, dried over anhydrous sodium sulfate and concentrated under reduced pressure. The crude was purified by flash column chromatography (220 g, EtOAc in hexanes, 5 to 100%) affording compound 7.4 (4.80 g, 75%) as a yellow oil. LC-MS: RT=1.91 min; MS cal.: 365.53; Mass found: [M+H]: 366.3. ¹H NMR (400 MHz, CDCl3) δ 7.71 (d, J=8.5 Hz, 2H), 7.48 (d, J=8.5 Hz, 2H), 3.66 (t, J=6.6 Hz, 2H), 3.11-3.02 (m, 4H), 2.43 (t, J=7.0 Hz, 2H), 1.73-1.36 (m, 12H), 0.86 (t, J=7.4 Hz, 6H).

8-(4-(N,N-Dipropylsulfamoyl)phenyl)octanoic acid (BT056). Palladium 10% wt on activated carbon (1.11 g, 13.1 mmol) was added to a solution of 4-(8-hydroxyoct-1-yn-1-yl)-N,N-dipropylbenzenesulfonamide 7.4 dissolved in MeOH (87.5 mL). After flushing (×3) the resulting mixture and adding hydrogen with a balloon, the reaction was stirred for 2 h at room temperature under hydrogen atmosphere. The mixture was filtered over Celite® (MeOH was used for the washing) and concentrated under reduced pressure affording crude alcohol (4.32 g, 89%) as a colourless oil. LC-MS: RT=1.98 min; MS cal.: 369.56; Mass found: [M+H]: 370.3. ¹H NMR (400 MHz, CDCl3) δ 7.69 (d, J=8.3 Hz, 2H), 7.28 (d, J=8.3 Hz, 2H), 3.63 (t, J=6.6 Hz, 2H), 3.10-2.99 (m, 4H), 2.71-2.58 (m, 2H), 1.72-1.44 (m, 10H), 1.32 (m, 6H), 0.86 (t, J=7.4 Hz, 6H). To a solution of the crude alcohol (974 mg, 2.64 mmol) in DCM (7 mL) containing Tempo (8.40 mg, 52.7 μmol) was added a solution of saturated aqueous NaHCO₃ (7 mL) containing KBr (47.5 mg, 395 μmol) and TBAC (75.5 mg, 264 μmol). The resulting mixture was then cooled down to 0° C. and a solution of NaOCl (6 mL, 8.57 mmol), saturated NaHCO₃ (3 mL) and brine (3 mL) was added dropwise over 15 min. LC-MS show the corresponding carboxilic acid and still have some starting material alcohol left. Another portion of NaOCl (6 mL) mixed with saturated NaHCO₃ (3 mL) and brine (3 mL) was added over 5 min and the reaction was stirred for 15 min. No more alcohol was observed by LC-MS. The reaction was diluted with water (10 mL) and DCM (10 mL) and the phases were separated. The organic material (DCM) was extracted with water (2×10 mL). The combined aqueous phases were treated at room temperature with 10% HCl until pH around 4. The organic material was then extracted with EtOAc (3×10 mL) from the resulting acid aqueous phase. Dried over anhydrous sodium sulfate, filtered and concentrated under reduced pressure. The crude was purified by flash column chromatography (50 g silica, EtOAc in hexanes, 10 to 100%) affording compound BT056 (795 mg, 79%) as a white solid. LC-MS: RT=1.93 min; Purity: 97%; MS cal.: 383.55; Mass found: [M−H]: 382.5. ¹H NMR (500 MHz, CDCl3) δ 7.70 (d, J=8.4 Hz, 2H), 7.28 (d, J=8.4 Hz, 2H), 3.12-3.01 (m, 4H), 2.71-2.59 (m, 2H), 2.35 (t, J=7.5 Hz, 2H), 1.63 (s, 3H), 1.55 (dq, J=14.9, 7.4 Hz, 4H), 1.34 (m, 3H), 0.87 (t, J=7.4 Hz, 6H).

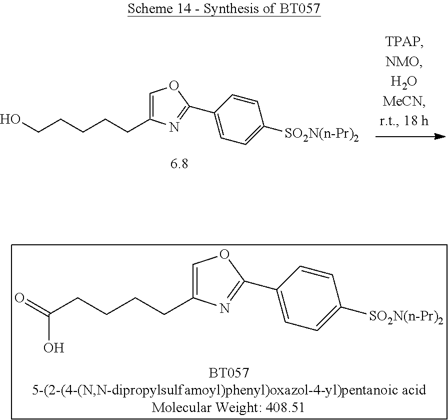

5-(2-(4-(N,N-Dipropylsulfamoyl)phenyl)oxazol-4-yl)pentanoic acid (BT057). Tetrapropylammonium perruthenate (111 mg, 317 μmol) was added to a solution of 6.8 (1.25 g, 3.17 mmol), 4-methylmorpholine N-oxide (3.44 g, 28.5 mmol) and H2O (85.5 μL, 4.75 mmol) dissolved in MeCN (12.7 mL). The reaction was stirred for 18 h at room temperature. The reaction was quenched with isopropyl alcohol (15 mL) and concentrated under reduced pressure. The crude was purified by flash column chromatography (50 g silica, 0 to 30% MeOH/DCM) affording BT057 as a white solid. LC-MS: RT=1.73 min; Purity: 96.4%; MS cal.: 408.51; Mass found: [M+H]: 409.0. ¹H NMR (500 MHz, CDCl3) δ 8.13 (d, J=8.7 Hz, 2H), 7.88 (d, J=8.7 Hz, 2H), 7.50 (s, 1H), 3.15-3.07 (m, 4H), 2.64 (t, J=6.7 Hz, 2H), 2.43 (t, J=7.0 Hz, 2H), 1.80-1.71 (m, 4H), 1.60-1.47 (m, 4H), 0.87 (t, J=7.4 Hz, 6H).

The following compounds were prepared using the procedures given above or by adapting the above-procedures using appropriate starting materials. As shown in the Table, each compound made gave the expected molecular ion or will give the expected molecular ion.

| Compound | Molecular | m/z |
|---|---|---|
| designation | weight | [M + H]⁺ |
| BT032 | 403.53 | 404 |
| BT137 | 444.54 | 445 |
| BT136 | 410.55 | 411 |
| BT135 | 410.57 | 411 |
| BT163 | 389.51 | 390 |
| BT160 | 369.52 | 370 |
| BT004 | 368.21 | 370 |
| BT005 | 298.14 | 299.92 |
| BT006 | 312.15 | 313.96 |
| BT007 | 285.10 | 286.89 |
| BT008 | 301.10 | 302.92 |
| BT009 | 249.31 | 250.85 |
| BT010 | 283.09 | 284.90 |
| BT011 | 345.41 | 346.91 |
| BT026 | 397.58 | |
| BT027 | 452.61 | 454.13 |
| BT028 | 412.54 | 413.31 |
| BT029 | 398.52 | 399.24 |
| BT030 | 388.57 | |
| BT031 | 388.57 | 385.99* |
| BT032 | 403.53 | 404 |
| BT033 | 403.53 | 405.10 |
| BT034 | 467.58 | 468.39 |
| BT041 | 383.55 | 384.3 |
| BT043 | 411.61 | 412.6 |
| BT052 | 496.07 | 496.4 |
| BT053 | 510.09 | 510.3 |
| BT054 | 368.58 | 369.2 |
| BT055 | 393.53 | 394.3 |
| BT056 | 383.55 | 382.5* |
| BT057 | 408.51 | 409.0 |
| BT058 | 352.41 | 351.4* |

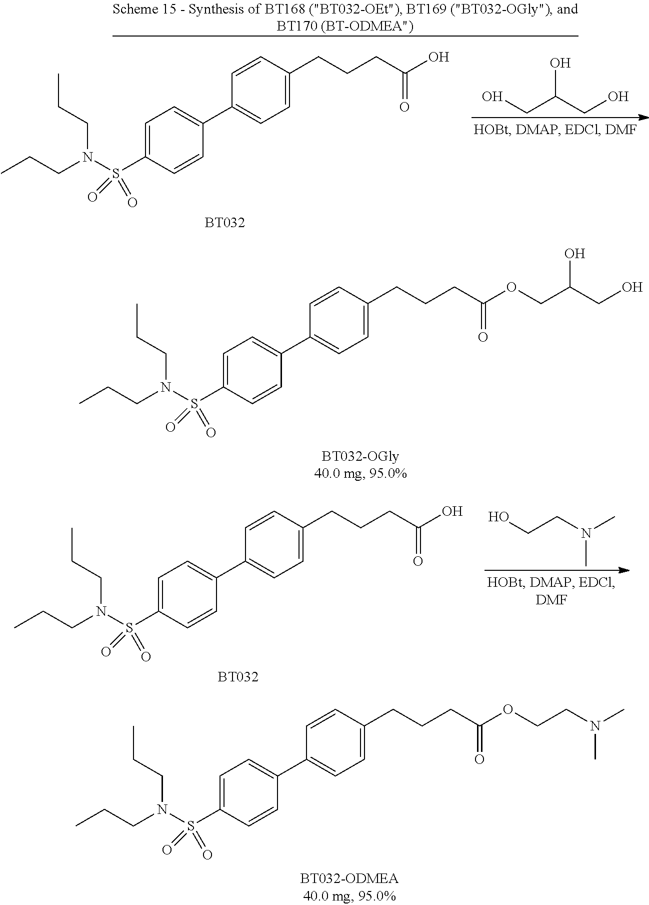

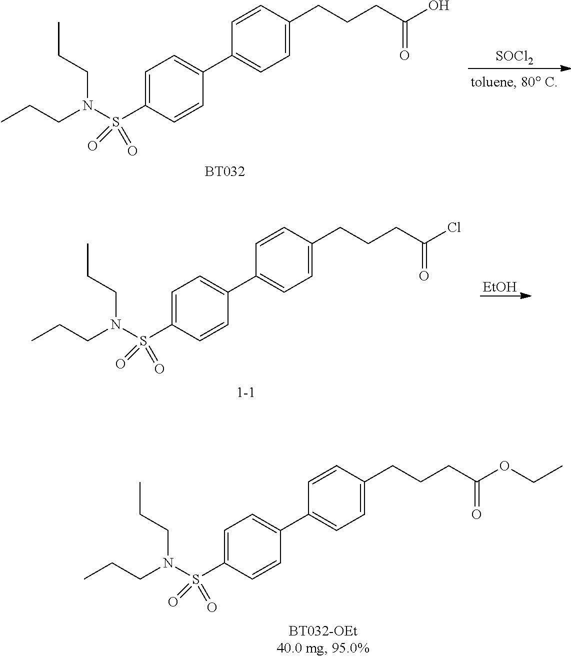

LC-MS or HPLC Method Method 1. MS instrument type: SHIMADZU LCMS-2020, Column: Kinetex EVO C18 30*2.1 mm, Sum, mobile phase A: 0.0375% TFA in water (v/v), B: 0.01875% TFA in Acetonitrile (v/v): 0.0 min 5% B→0.8 min 95% B→1.2 min 95% B→1.21 min 5% B→1.55 min 5% B, flow rate: 1.5 mL/min, oven temperature: 50° C.; UV detection: 220 nm & 254 nm.

LC-MS or HPLC Method Method 2. MS instrument type: SHIMADZU LC-20AD, Column: Kinetex C18 LC Column 4.6×50 mm, Sum, mobile phase A: 0.0375% TFA in water (v/v), B: 0.01875% TFA in Acetonitrile (v/v), gradient: 0.0 min 10% B→2.4 min 80% B→3.7 min 80% B→3.71 min 10% B→4.0 min 10% B, flow rate: 1.5 mL/min, oven temperature: 50° C.; UV detection: 220 nm&215 nm&254 nm.

### General Procedure for Preparation of Compound 1-1:

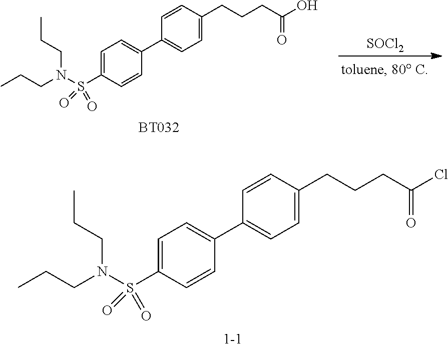

To a mixture of BT032 (3.00 g, 7.43 mmol, 1.00 eq) in toluene (30 mL) was added SOCl₂ (4.92 g, 41.4 mmol, 3.00 mL, 5.56 eq). Then the reaction mixture was stirred at 80° C. for 2 hrs. LCMS (EW31573-1-P1A2) showed BT032 was consumed mostly and desired MS was detected. The reaction mixture was concentrated under reduced pressure to give Compound 1-1 (3.00 g, 7.11 mmol, 95.6% yield) as yellow oil. LCMS method 1, RT=1.110 min, MS+11=432.3.

### General Procedure for Preparation of Compound BT032-OEt:

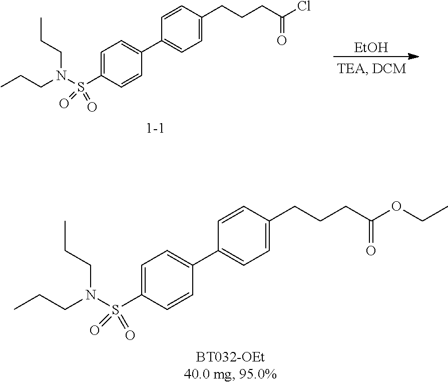 [SMILES: "EtOH" ; CCCN(CCC)S(=O)(=O)c1ccc(-c2ccc(CCCC(=O)Cl)cc2)cc1 ; "1-1" ; CCCN(CCC)S(=O)(=O)c1ccc(-c2ccc(CCCC(=O)OCC)cc2)cc1]

To a mixture of EtOH (54.6 mg, 1.18 mmol, 1.00 eq) and TEA (120 mg, 1.18 mmol, 165 μL, 1.00 eq) in DCM (5.00 mL) was added dropwise compound 1-1 (500 mg, 1.18 mmol, 1.00 eq) at 0° C. The reaction mixture was stirred at 20° C. for 0.5 hr. LCMS (EW16077-223-P1A) showed the starting material was consumed and desired MS was detected. The reaction mixture was concentrated under reduced pressure and purified by reversed-phase HPLC (column: 3_Phenomenex Luna C18 75*30 mm*3 um; mobile phase: [water (HCl)-ACN]; B %: 68%-88%, 6 min). BT032-OEt (347 mg, 804 umol, 1.00 eq) was obtained as a brown gum. LCMS method 1, RT=1.112 min, MS+1=432.3 [M+H⁺]; LCMS method 1, RT=1.073 min, MS+1=432.0 [M+H⁺]; HPLC method 2, RT=2.755 min; ¹H NMR: (400 MHz DMSO-d₆) δ 7.88-7.83 (m, 4H), 7.87 (d, J=8.0 Hz, 2H), 7.33 (d, J=8.0 Hz, 2H), 4.08-4.03 (m, 2H), 3.07-3.03 (m, 4H), 2.67-2.65 (m, 2H), 2.31-2.30 (m, 2H), 1.88-1.81 (m, 2H), 1.52-1.47 (m, 4H), 1.20-1.16 (m, 3H), 0.84-0.81 (m, 6H).

### General Procedure for Preparation of Compound BT032-OGly:

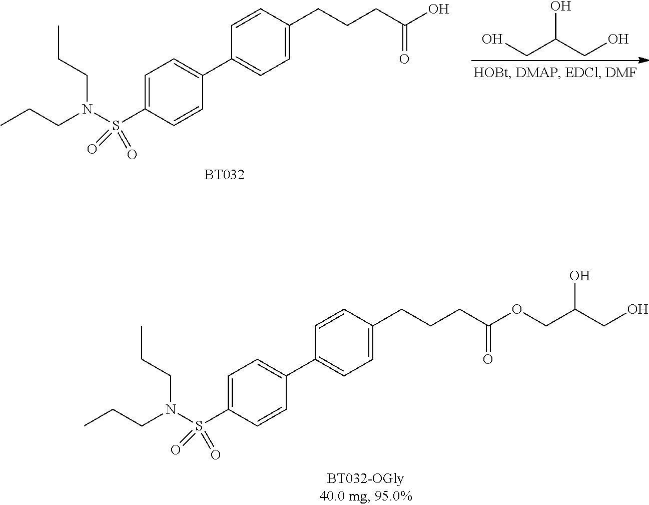

A mixture of BT032 (500 mg, 1.24 mmol, 1.00 eq), GLYCEROL (1.14 g, 12.4 mmol, 928 μL, 10.0 eq), HOBt (201 mg, 1.49 mmol, 1.20 eq), DMAP (182 mg, 1.49 mmol, 1.20 eq), EDCI (285 mg, 1.49 mmol, 1.20 eq) in DMF (5.00 mL) was stirred at 25° C. for 5 hrs. LCMS (EW31742-5-P1A1) showed the desired compound was detected. The reaction mixture was filtered. The filtrate was purified by reversed-phase HPLC (column: 3_Phenomenex Luna C18 75*30 mm*3 um; mobile phase: [water (HCl)-ACN]; B %: 51%-71%, 6 min) to give BT032-Gly (450 mg, 942 umol, 76.0% yield) as a brown gum. LCMS method 1, RT=0.855 min, MS+1=478.3 [M+H⁺]; LCMS method 1, RT=0.926 min, MS+1=478.0 [M+H⁺]; HPLC method 2, RT=2.368 min; ¹H NMR: 400 MHz (DMSO-d₆) δ 7.89-7.82 (m, 4H), 7.67 (d, J=8.4 Hz, 2H), 7.34 (d, J=8.0 Hz, 2H), 4.88 (d, J=8.0 Hz, 1H), 4.76-4.74 (m, 1H), 4.07-4.03 (m, 1H), 3.94-3.89 (m, 1H), 3.65-3.61 (m, 1H), 3.07-3.03 (m, 4H), 2.67-2.64 (m, 2H), 2.35-2.31 (m, 2H), 1.88-1.85 (m, 2H), 1.53-1.44 (m, 4H), 0.84-0.80 (m, 6H).

### General Procedure for Preparation of BT032-ODMEA:

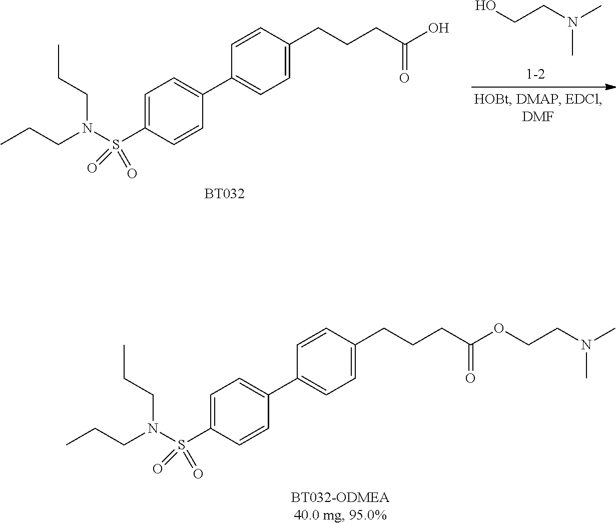

A mixture of BT032 (500 mg, 1.24 mmol, 1.00 eq), compound 1-2 (133 mg, 1.49 mmol, 149 uL, 1.20 eq), HOBt (201 mg, 1.49 mmol, 1.20 eq), DMAP (182 mg, 1.49 mmol, 1.20 eq), EDCI (285 mg, 1.49 mmol, 1.20 eq) in DMF (5.00 mL) was stirred at 25° C. for 5 hrs. LCMS (EW31742-6-P1A1) showed the desired compound was detected. The reaction mixture was filtered. And then, the filtrate was purified by reversed-phase HPLC (column: 3_Phenomenex Luna C18 75*30 mm*3 um; mobile phase: [water (HCl)-ACN]; B %: 37%-57%, 6 mins) to give BT032-ODMEA was obtained as a brown gum. LCMS method 1, RT=0.767, MS+1=475.4 [M+H⁺]; LCMS method 1, RT=0.880 min, MS+1=475.4 [M+H⁺]; HPLC method 2, RT=2.221 min ¹H NMR: 400 MHz (DMSO-d₆) δ 7.89-7.83 (m, 4H), 7.67 (d, J=8.4 Hz 2H), 7.34 (d, J=8.0 Hz 2H), 4.11-4.08 (m, 2H), 3.07-3.03 (m, 4H), 2.65 (t, J=7.5 Hz 2H), 2.47-2.46 (m, 2H), 2.33 (t, J=7.3 Hz 2H), 2.17 (s, 6H), 1.90-1.82 (m, 2H), 1.52-1.46 (m, 4H), 0.83 (t, J=7.3 Hz, 6H).

### Example 2: Inhibition of Inflammasome Activation by Compounds of the Present Technology

This example demonstrates the efficacy of the compounds of the present technology in inhibiting NLRP3 inflammasome activation in vitro, and that the compounds of the present technology exhibit a dose-responsive inhibition of inflammasome activation as assessed by IL-1β secretion.

In vitro stimulation of murine macrophages. Immortalized wild-type C57BL/6 bone-marrow derived macrophages (iBMDMs) were grown in DMEM supplemented with 10% heat inactivated FBS and 2 mM glutamine. iBMDMs were seeded in 96-well plates 24 hours prior incubation with lipopolysaccharide (LPS; 100 ng/mL) for 3 hours. Cells were then incubated with a control probenecid compound (probenecid dissolved in DMSO (Prob/D), probenecid dissolved in PBS (Prob/P)) or one of the probenecid analogs of the present technology (BT032, BT132, BT133, BT134, BT135, BT136, BT137, BT138, BT139, or BT140) at concentrations of 300 μM, 150 μM, 30 μM, or 3 μM 1 hour prior to stimulation with NLRP3 inflammasome activators, silica (250 pg/mL) or nigericin (6 μM). After an additional 6 hours, cell supernatants were collected and levels of IL-1β were quantified by ELISA. These experiments were run in triplicate three times.

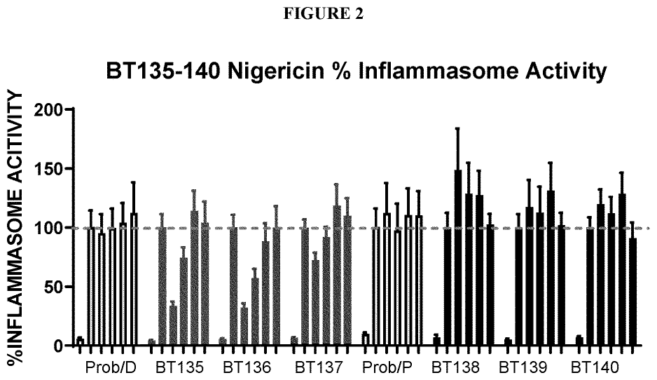

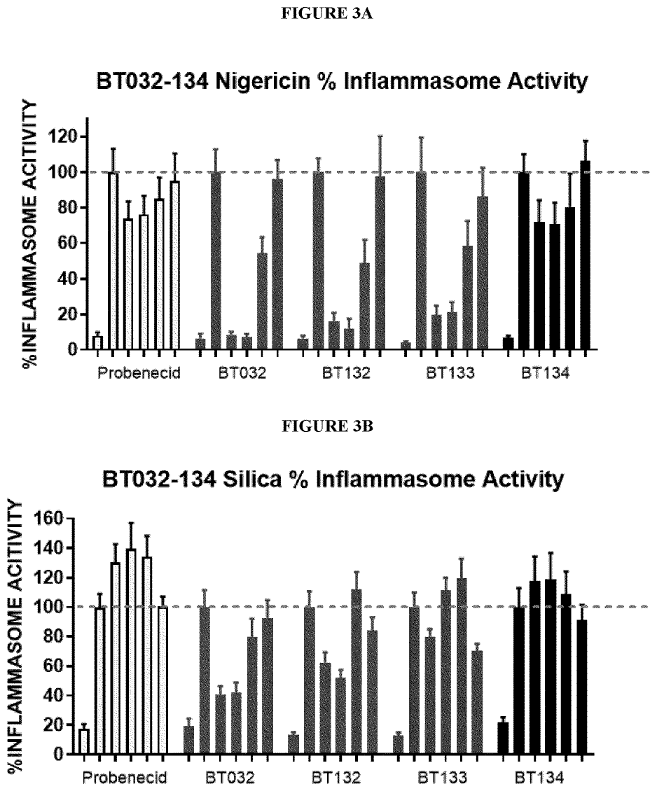

Results. As shown by FIG. 2 and FIGS. 3A and 3B, treatment with the probenecid analogs of the present technology reduced IL-1β secretion in a dose-dependent manner in response to different NLRP3 stimuli (i.e., silica and nigericin). Overall, probenecid analogs BT032, BT132, BT133, and BT136 are more potent than the control probenecid compounds.

Accordingly, these results demonstrate that probenecid analogs of the present technology are useful in methods for inhibiting NLRP3 inflammasome activation and for treating or preventing inflammasome-mediated dermatological diseases or conditions.

### Example 3: Inhibition of NLRP1 and NLRP3 Inflammasomes by Probenecid Analogs of the Present Technology

This example demonstrates the efficacy of the compounds of the present technology in inhibiting NLRP1 and NLRP3 inflammasome activation in vitro, and that the compounds of the present technology exhibit a dose-responsive inhibition of inflammasome activation as assessed by IL-1β secretion.

IC50 curves. Immortalized BMDMs grown in DMEM/10% FCS, 2 mM glutamine at 5% CO2 were seeded at 4×10⁴ cells in 96 well format, 20 h prior to priming with 100 ng/ml LPS E. coli 055:B5 for a further 3 h. Macrophages were treated with drug (3.9-350 μM) or vehicle (DMSO) in serum-free media for 60 mins prior to challenge with nigericin (3 μM) for 120 mins. Cultured supernatants were assayed for secreted IL-1β by ELISA according to manufacturer's instruction.

In vitro stimulation of human macrophages. Immortalized BMDMs grown in DMEM/10% FCS, 2 mM glutamine at 5% CO2 were seeded at 4×10⁴ cells in 96 well format, 20 h prior to priming with 100 ng/ml LPS E. coli 055:B5 (except for LPS (B4) priming done with Pam₃Cys; 100 ng/ml) for a further 3 h. Macrophages were treated with vehicle (DMSO), BT032 (20, 100 μM), or MCC950 (5 μM) where indicated in serum-free media for 60 mins prior to challenge with NLR agonists; nigericin (3 μM; 120 mins), monosodium urate crystals; MSU (250 pg/ml; 6 h), silica MSU (250 μg/ml; 6 h), L18-MDP (100 μg/ml; 16 h), LPS Serotype 0111:B4 (2 μg; 16 h), poly dA:dT (1 pg; 6 h) and Flagellin (200 pg; 6 h) all complexed with Lipofectamine 2000. Cultured supernatants were assayed for secreted IL-1β by ELISA according to manufacturer's instructions.

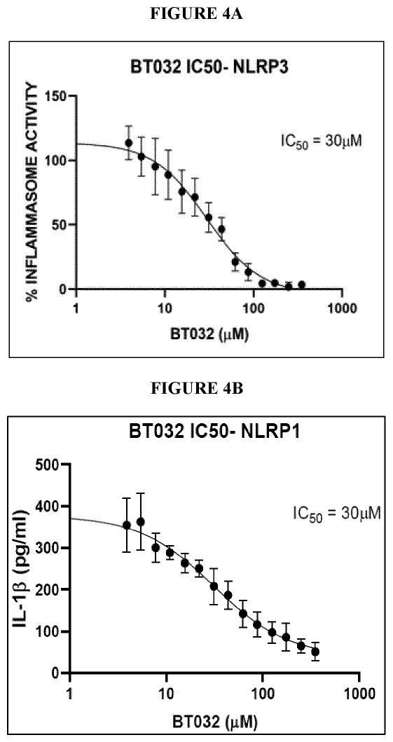

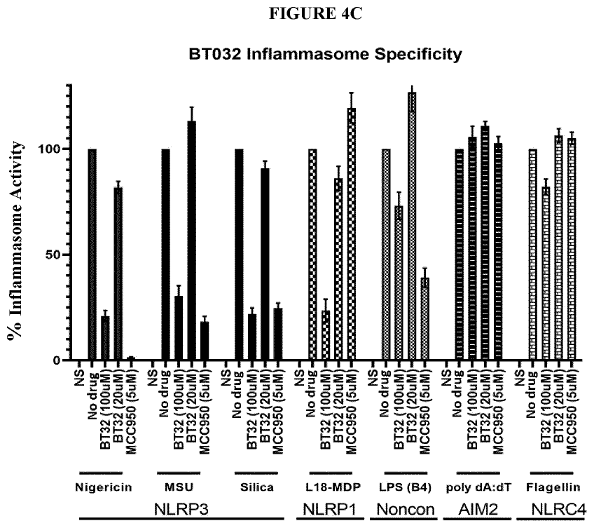

Results. As shown by FIGS. 4A and 4B, the half maximal inhibitory concentration (IC₅₀) for BT032 inhibition of NLRP3- and NLRP1-mediated inflammasome activity, respectively, is 30 μM. As shown by FIG. 4C, treatment of macrophages with BT032 specifically dose-dependently inhibited NLRP3 (Nigericin, Monosodium Urate; MSU, and Silica)- and NLRP1 (L18-MDP)-induced inflammasome activation. BT032, however, had limited impact upon non-canonical inflammasome activity (LPS (B4)) and no effect upon both AIM2 (poly dA:dT) and NLRC4 (Flagellin)-mediated inflammasome activation. Importantly, while the specific NLRP3 inhibitor MCC950 inhibited nigericin, MSU, and silica inflammasome activation, unlike BT032, it had no impact upon NLRP1 activity. These findings demonstrate that probenecid analogs of the present technology (e.g., BT032) are specific NLRP1 and NLRP3 inhibitors.

Accordingly, these results demonstrate that probenecid analogs of the present technology, such as BT032, are useful in methods for inhibiting NLRP3 and NLRP1 inflammasome activation and for treating or preventing inflammasome-mediated dermatological diseases or conditions.

### Example 4: BT032 Inhibits Nigericin-Induced ASC Speck Formation

This example demonstrates the efficacy of the compounds of the present technology in inhibiting the formation of ASC (apoptosis-associated speck-like protein containing a caspase activating and recruitment domain) specks.

Methods. NLRP3-deficient immortalized macrophages stably expressing ASC-cerulean and NLRP3 were seeded in 8-well Ibidi chamber slides (2×10⁵/ml) 24 h prior to stimulation. Macrophages were treated with BT032 (350 μM) or vehicle (DMSO) in serum-free media for 60 mins prior to challenge with nigericin (3 μM; 90 mins). 10 mins prior to harvesting, cells were treated with Hoechst 33342, washed and fixed in 4% paraformaldehyde and stored in PBS. Six random fields were imaged at 60× magnification with 40 z planes. Images are deconvoluted z stacks by overlapping scanning processed using ImageJ. ASC-cerulean specks were counted for each field and as percentage of specks per field and represented as a percentage of total Hoechst positive cells.

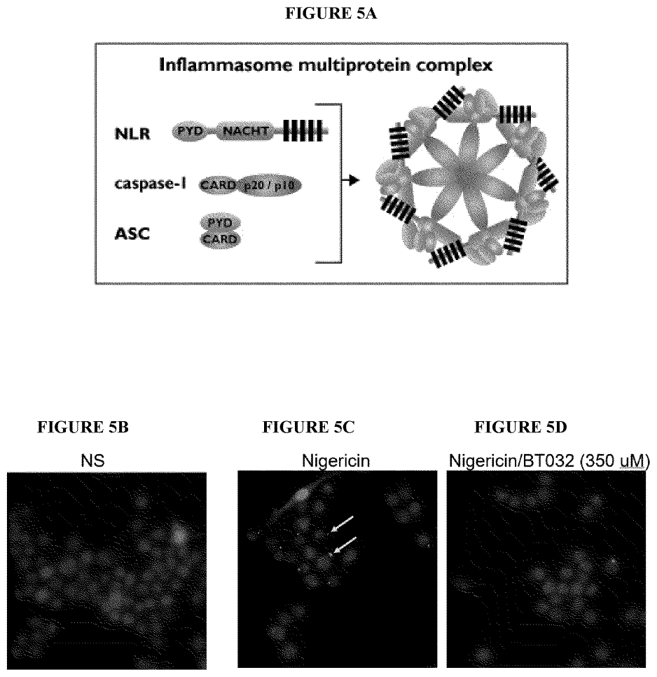

Results. As shown by FIGS. 5B-5E, while ASC appears diffuse in the cytosol of unstimulated cells (FIG. 5B), upon stimulation with nigericin, ASC staining appears intense and punctate, indicative of inflammasome activation in approximately 20% of cells (FIG. 5E). However, when macrophages are pretreated with BT032, they demonstrate substantially reduced ASC specks in cells (FIGS. 5D and 5E) indicating that BT032 inhibited inflammasome oligomeric formation. Importantly, as ASC-cerulean macrophages do not require priming for activation, this result also demonstrates that BT032 specifically inhibits formation of the oligomeric inflammasome complex and not priming (i.e., NF-κB activation and upregulation of IL-1β and NLRP3).

Accordingly, these results demonstrate that probenecid analogs of the present technology, such as BT032, are useful in methods for inhibiting inflammasome formation and for treating or preventing inflammasome-mediated dermatological disease or conditions.

### Example 5: Lipopolysaccharide (LPS) Model for NLRP3 Inflammation

This example demonstrates the efficacy of the compounds of the present technology in inhibiting NLRP3 inflammasome activation following acute intraperitoneal (i.p.) challenge with LPS.

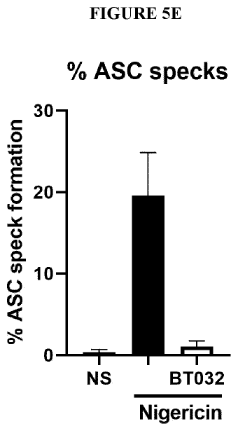

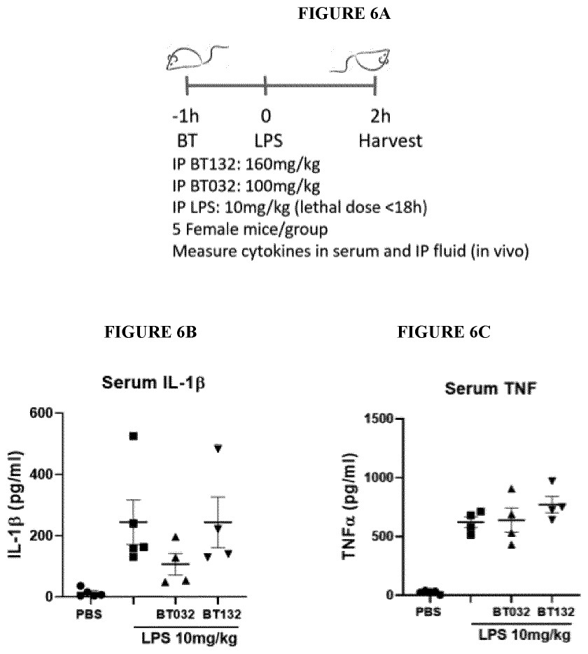

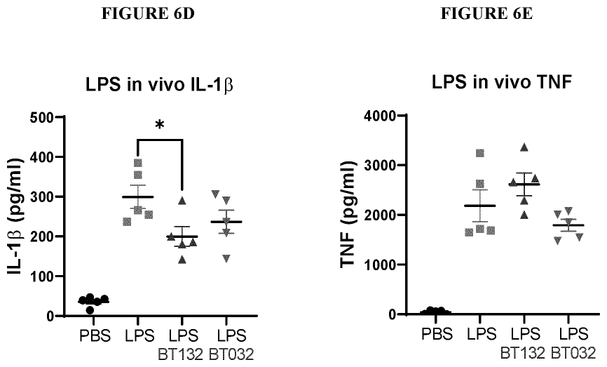

In vivo LPS challenge. As shown in FIG. 6A, female C57BL/6 mice (6-8 weeks old) were injected intraperitoneally (i.p.) with PBS or drug (i.e., BT132 or BT032) in PBS 1 hour before i.p. injection of 10 mg/kg LPS Escherichia coli 055:B5 (Sigma Aldrich) or PBS. After 2 hours, the mice were sacrificed and serum and i.p. fluid levels of IL-1β and TNF-α were measured by ELISA.

Results. Overall, the results show that the probenecid analogs of the present technology are effective in methods for reducing serum and i.p. fluid IL-1β production (FIGS. 6B and 10D). In addition, the results demonstrate that the effect is NLPR3-specific in that serum and i.p. fluid TNF-α levels were not affected (FIGS. 6C and 6E).

Accordingly, these results demonstrate that probenecid analogs of the present technology, such as BT032 and BT132, are useful in methods for inhibiting inflammasome activation and for treating or preventing inflammasome-mediated dermatological disease or conditions.

### Example 6: Inhibition of NLRP1 Inflammasomes by Probenecid Analogs of the Present Technology

This example demonstrates the efficacy of the compounds of the present technology in inhibiting NLRP1 inflammasome activation in vitro, and that the compounds of the present technology exhibit a dose-responsive inhibition of inflammasome activation as assessed by IL-1β secretion.

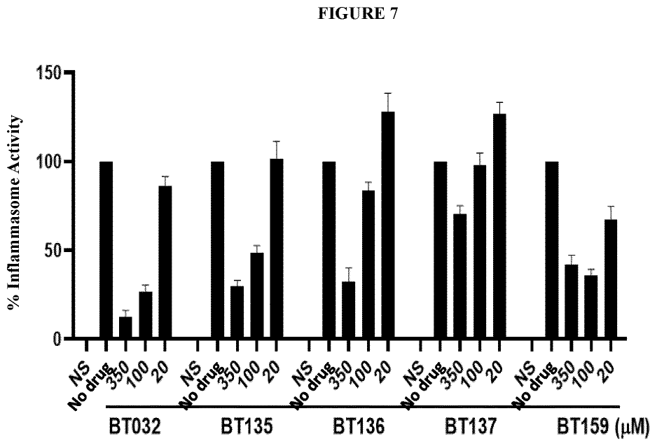

In vitro stimulation of human macrophages. Immortalized BMDMs grown in DMEM/10% FCS, 2 mM glutamine at 5% CO2 were seeded at 4×10⁴ cells in 96 well format, 20 h. Macrophages were treated with BT032 (20, 100, 350 μM), BT135 (20, 100, 350 μM), BT136 (20, 100, 350 μM), BT137 (20, 100, 350 μM), or BT159 (also known as BT052) (20, 100, 350 μM) where indicated in serum-free media for 60 mins prior to challenge with NLRP1 agonist, L18-MDP (100 μg/mL; 16 hours) (NS=non-stimulated cells). Cultured supernatants were assayed for secreted IL-1β by ELISA according to manufacturer's instructions and are represented in FIG. 7 as the % maximal activation of untreated macrophages (“no drug”). Pooled results of 3 independent experiments were conducted in triplicate where activity was normalized as percentage of activity as related to the DMSO-treated control cells (“no drug”) and non-stimulated (NS) cells.

Results. As shown by FIG. 7, treatment of macrophages with BT032, BT135, BT136, and BT159 (also referred to as BT052 herein) dose-dependently inhibited NLRP1 (L18-MDP)-induced inflammasome activation. These results demonstrate that probenecid analogs of the present technology (e.g., BT032, BT135, BT136, BT159) are NLRP1 inhibitors.

Accordingly, these results demonstrate that probenecid analogs of the present technology, such as BT032, BT135, BT136, BT159, are useful in methods for inhibiting NLRP1 inflammasome activation and for treating or preventing inflammasome-mediated dermatological diseases or conditions.

### Example 7: Probenecid Analogs of the Present Technology for the Prevention and Treatment of Inflammasome-Mediated Dermatological Diseases or Conditions—ZAKα-Driven Ribotoxic Stress Response Pathway (RSR) Inhibition Model

This example demonstrates the capability of the probenecid analogs of the present technology to reduce inflammasome-mediated dermatological diseases or conditions and hyperinflammation in vitro due to NLRP1 activation by the ZAKα-driven RSR pathway induced by ultraviolet B (UVB) or UVA irradiation or ZAKα-activating toxins (anisomycin (ANS), doxyvinenol (DON), hygromycin (HYGRO)) in human keratinocytes.

Cell culture and chemicals. Immortalized human keratinocytes (N/TERT-1 or N-TERT) are obtained from a commercial source. Primary human keratinocytes are derived from the skin of healthy donors and obtained with informed consent.

Cells are incubated with a control probenecid compound (probenecid dissolved in DMSO (Prob/D), probenecid dissolved in PBS (Prob/P)) or one of the probenecid analogs of the present technology (BT032, BT132, BT133, BT134, BT135, BT136, BT137, BT138, BT139, or BT140) at concentrations of 300 μM, 150 μM, 30 μM, or 3 μM prior to stimulation with ultraviolet B (UVB) or UVA irradiation or ZAKα-activating toxins (anisomycin (ANS), doxyvinenol (DON), or hygromycin (HYGRO)).

UVB and UVA irradiation. For UVA and UVB irradiation experiments, cells are seeded in 6 cm dishes, incubated for 24 hours and washed once in phosphate buffered saline (PBS) pH 7.4 before being exposed to indicated dose of irradiation using a BIO SUN microprocessor controlled, cooled UV irradiation system (or a similar apparatus). After exposure, PBS is replaced by keratinocyte medium and cells are incubated for an indicated time.

Cytokine analysis. To measure secreted cytokine and chemokine levels, a human IL-1β enzyme linked immunosorbent assay (ELISA) kit (such as BD, #557953), human IL-6 ELISA kit (such as R&D Systems, D6050), and a human IL-8/CXCL8 ELISA kit (such as R&D Systems, D8000C) will be used.

Microscopy. Images of ASC-GFP specks are acquired in 3 random fields in 4′,6-diaminidino-2-phenylindole (DAPI, 358 nm/461) and GFP (469/525 nm) channels using EVOS microscope (such as FL Auto M5000, #AMF5000) according to the manufacturer's protocol. Quantification method of ASC-GFP specks has been previously described.

Flow cytometry-based quantification of inflammasome assembly. To quantify the assembly of ASC-EGFP specks or recruitment of C1C-EGFP to ASC specks (“C1C specks”), human keratinocytes are treated in 24-wells and analyzed by flow cytometry. Cells are seeded and cultivated overnight. Cells are stimulated for 6 to 20 h in DMEM (10% FBS) (HEK 293T) or keratinocyte SFM (BPE, EGF, CaCl₂), 100 μM BT032) (N/TERT-1), harvested by trypsinization, fixed in 4% formaldehyde, and analyzed using BD FACSCanto and BD LSRFortessa SORP flow cytometers, recording area, width, and height of the EGFP signal of single cells. Where indicated, cells are pre-treated with probenecid analogs of the present technology (e.g., BT032) for 30 min before stimulation. UV stimulation is performed by irradiating cells in tissue culture plates (without lid) in a Bio-Link UVB irradiation system equipped with 5×8 Watt T-8.M tubes emitting UVB at 213 nm for 3 min, followed by cultivation for 20 h. For infection experiments, cells are infected for 1 h in serum-free medium at the indicated multiplicity of infection (MOI), ranging from 1-50. Medium is subsequently replaced with full medium (with indicated inhibitors, e.g. BT032), and cells are cultivated for another 19 h. To transiently overexpress proteins in HEK-based vector reporter cells, cells are transfected using Lipofectamine 2000 or PEI Max, medium is replaced after 4 h, and cells are harvested 20 h post transfection. Dox-inducible expression in lentivirus-generated cell lines is initiated by cultivating cells in 1 μg/mL dox for 6 to 20 h. Fixed and permeabilized cells are stained with anti-HA (1:1000), anti-FLAG (1:300), anti-phospho-p38 (1:400), anti-gamma H2A.X (phosphor S139) (1:500), anti-dsRNA (1:500), or anti-VSV G (1:1,000) in Intracellular Staining Permeabilization Wash Buffer combined with Alexa Fluor (AF) 405, or AF647-coupled, highly cross-absorbed secondary antibodies.

Cytokine quantification by HTRF. To quantify IL-1β secretion, N/TERT-1 derived cells are stimulated for flow cytometry experiments in the absence and presence of BT032. IL-1β is quantified using the Human IL 1 beta HTRF kit according to the manufacturer's instructions. Supernatants for the quantification of IL-1β levels after inducible expression of VHL-VHH fusions are collected from 5×10⁴ cells per well in 96-well plates.

Alphavirus-induced NLRP1 activation. Human keratinocytes will be infected with the model alphavirus SFV, which is reported to activate NLRP1 inflammasomes. To study the effects of the probenecid analogs of the present technology (e.g., BT032) on NLRP1 activation in response to viral infection, HEK^{NLRP+ASC}, HEK^{NLRP3+ASC} and N/TERT-1^{C1C-EGFP} cells are infected with SFV, the closely related Sindbis virus (SINV), as well as vesicular stomatitis virus (VSV), a negative-sense single-stranded RNA virus. Cells are treated with the probenecid analogs of the present technology (e.g., BT032). Infection is quantified by staining for dsRNA (SFV, SINV) or the VSV-G protein, and only infected cells are included in the flow cytometry analysis of inflammasome activation.

Results. It is anticipated that the probenecid analogs of the present technology (e.g., BT032) will inhibit IL-1β secretion levels in human keratinocytes following UVB irradiation, and/or will reduce UVB-induced accumulation of detergent insoluble ASC oligomers in keratinocytes, and/or will reduce inflammasome assembly in keratinocytes as measured by ASC speck formation.

It is further anticipated that the probenecid analogs of the present technology (e.g., BT032) will abrogate UVA with either 4-SU and BrdU-induced IL-1β p17 secretion 24 hours post-irradiation in human skin keratinocytes.

It is also anticipated that the probenecid analogs of the present technology (e.g., BT032) will abrogate ANS, HYGRO, and/or DON-induced inflammasome-driven IL-1β section in human keratinocytes after a 24 hour incubation. It is further anticipated that treatment of keratinocytes infected with viruses will reduce the assembly of ASC specks in the keratinocytes post-infection.

Accordingly, these results will demonstrate that the probenecid analogs of the present technology are effective in methods for preventing and treating inflammasome-mediated dermatological diseases or conditions.

### Example 8: Probenecid Analogs of the Present Technology for the Prevention and Treatment of Inflammasome-Mediated Dermatological Disease—Psoriasis Model

This example will demonstrate the capability of the probenecid analogs of the present technology (e.g., BT032) to treat or prevent psoriasis in subjects.

### BACKGROUND

Psoriasis is a chronic inflammatory skin disease characterized by IL-17-mediated immune responses, and p38 is known to be highly activated in the psoriatic epidermis. In this example, a p38 activator, anisomycin, is applied daily to murine skin. The probenecid analogs of the present technology (e.g., BT032) are applied to murine psoriatic models topically or to human psoriatic skin specimens ex vivo.

### Methods

Mice. Eight- to 12-week old female mice are used in this study. All animals are of the C57BL/6 genetic background. Animals are maintained under specific pathogen-free conditions.

Anisomycin treatment. Anisomycin solution (2 mg/mL in 99.5% ethanol) is applied to the inner and outer sides of a murine ear once daily at a dosage of 10 μL per side of the ear.

Cell culture. Murine epidermis is harvested and subjected to keratinocyte preparation performed by using the following methods. In general, full-thickness skin is obtained from newborn mice. Skin samples are floated and incubated in Dispase II (1 U/mL; Roche, Mannheim, Germany) overnight at 48° C. The epidermis is then separated from the dermis, cut into pieces, and incubated in 0.05% trypsin for 5 minutes. Cells are cultured in low-calcium medium (0.05 mmol/L Ca21) using Eagle minimum essential medium (Lonza, Basel, Switzerland) with chelated FCS. Cells are seeded on 12-well plates at a density of 3 3 105 cells per well for RNA extraction. For cell-signaling experiments, cells are serum starved for 24 hours before stimulation. Anisomycin (50 ng/mL) and BT032 (50 ng/mL) are dissolved in dimethyl sulfoxide (DMSO) and used for cell stimulation. Normal human epidermal keratinocytes (NHEKs) are inoculated at a concentration of 2500 cells/cm² in culture medium, HuMedia-KG2 (Kurabo) supplemented with insulin (10 mg/mL), human epidermal growth factor (0.1 ng/mL), hydrocortisone (0.5 mg/mL), bovine pituitary extract (0.4% vol/vol), gentamicin (50 mg/mL), and amphotericin B (50 ng/mL). The culture was maintained at 37° C. in a 95% O₂/5% CO₂ humidified chamber. Anisomycin (50 ng/mL) and BT032 (50 ng/mL) are dissolved in DMSO and used for cell stimulation. rIL-17A (200 ng/mL; R&D Systems, Minneapolis, Minn) was used to stimulate primary keratinocytes, and BT032 (500 ng/mL) in DMSO was used as an inhibitor.

Human samples. Biopsy specimens of psoriatic lesions are obtained from about 10 patients after obtaining informed consent. Two adjacent biopsy specimens are taken from each patient. All specimens are cut at the upper dermis, and the epidermal side is used for the experiment. As a medium, HuMedia-KB2 (Kurabo) containing insulin, human epidermal growth factor, bovine pituitary extract, and gentamicin/amphotericin B is used, according to the manufacturer's protocol. One specimen is cultured with vehicle (DMSO), and the adjacent one was cultured with BT032 (500 ng/mL) for 12 hours.

Histological analyses. For histologic examination, ear skin samples are fixed with 10% formalin and embedded in paraffin. Sections along the median plane of the ear with a thickness of 5 μm are prepared and subjected to staining with hematoxylin and eosin (H&E). Images of two consecutive microscopic fields of H&E-stained sections are obtained using a digital microscope. Time course changes in ear thickness are measured. Thickness is measured daily before treatment.

Flow cytometry analyses. Flow cytometric analysis is performed by the following methods. Ear samples are collected as 8-mm skin biopsy specimens and separated into dorsal and ventral sides. They are then cut into pieces and incubated for 60 min at 37° C. in a solution of complete RPMI containing 100 μg/mL of DNase I (Sigma-Aldrich, St. Louis, MO) and 2 mg/mL of Liberase TL (Roche, Indianapolis, IN) to obtain a homogeneous cell suspension. The cell suspensions are filtered using a 40-μm cell strainer and stained. For analysis of intracellular cytokine production, cell suspensions are obtained in the presence of 10 μg/mL of brefeldin A (Sigma-Aldrich), fixed with Cytofix Buffer, and permeabilized with Perm/Wash Buffer according to the manufacturer's protocol (BD Biosciences, San Jose, CA). The following antibodies are used: antibody to mouse CD4-FITC (RM4-5), anti-CD11c-BV605 (N418), anti-MHC class II-FITC (M5/114.15.2), anti-76 TCR-FITC/PB (GL3), and anti-Ly6G-FITC (1A8) (all from BioLegend, San Diego, CA, USA); anti-CD45-PE-Cy7/PB (30-F11), anti-76 TCR-FITC (GL3), and anti-IL-17A-PE (TC11-18H10) (all from BD Biosciences). Flow cytometry is performed using an LSR Fortessa (BD Biosciences), and data are analyzed with FlowJo software (TreeStar, San Carlos, CA).

RNA isolation and the quantitative RT-PCR analysis. Total RNA is isolated using Trizol (Invitrogen, Carlsbad, CA) or RNeasy kits (Qiagen, PL Venlo, Netherlands) according to the manufacturer's protocol. Complementary DNA is reverse transcribed using a Prime Script RT reagent kit (Takara Bio, Shiga, Japan). The quantitative RT-PCR is performed by monitoring the synthesis of double-stranded DNA during the various PCR cycles with SYBR Green I (Roche, Basel, Switzerland) and the LightCycler real-time PCR apparatus (Roche), according to the manufacturer's instructions. All primers are obtained from Greiner Japan (Tokyo, Japan).

Immunofluorescence analysis. Frozen skin or embryonic sections and cultured keratinocytes are fixed in 4% paraformaldehyde/phosphate-buffered saline (PBS). After 30 min of blocking using 10% goat sera, samples are incubated overnight at 4° C. with primary antibodies, p-p38 (Cell Signaling Technology, Danvers, MA, #9211). Samples are rinsed with PBS before and after incubation with secondary antibodies conjugated to Alexa-488 (Invitrogen) for 60 min at room temperature. Nuclei are stained with ProLong™ Diamond Antifade Mountant with 4′,6-diamidino-2-phenylindole (DAPI) (Invitrogen).

Murine BMDCs. For BMDC culture, 5×10⁶ bone marrow cells generated from wild-type mice are cultured in 10 mL of complete RPMI (RPMI 1640; Sigma-Aldrich) containing 10% heat-inactivated fetal cow serum (FCS) (Invitrogen), 50 μM of 2-mercaptoethanol, 2 mM of 1-glutamine, 25 mM of N-2-hydroxyethylpiperazine-N′-2-ethanesulfonic acid, 1 mM of nonessential amino acids, 1 mM of sodium pyruvate, 100 U/ml of penicillin, and 100 μg/ml of streptomycin supplemented with 10 ng/ml of recombinant murine GM-CSF (PeproTech, Rocky Hill, NJ) for 5 days. Anisomycin (50 ng/ml) is dissolved in DMSO and used for cell stimulation.

Microarray analysis. Total RNA prepared from the ears of mice treated with anisomycin for 5 consecutive days (n 3) is subjected to microarray analysis. Total RNA from skin samples is immediately isolated with Trizol according to the manufacturer's protocol. An amplified sense-strand DNA product is synthesized with an Ambion WT Expression Kit (Life Technologies, Carlsbad, CA), then fragmented and labelled with a WT Terminal Labelling and Controls Kit (Affymetrix, Santa Clara, CA) and hybridized to a Mouse Gene 1.0 ST Array (Affymetrix). Microarray analyses are carried out in R Bioconductor packages. Quality control of microarray chips is carried out with standard quality control metrics and R package microarray quality control. Images are scrutinized for spatial artifacts using Harshlight. Expression measures are obtained with the GCRMA algorithm. Expression values are modeled with mixed-effect models, with the inductor as the fixed factor and a random effect for each subject. Fold changes (FCHs) for comparisons of interest are estimated, and hypothesis testing is conducted with contrasts under the general framework for linear models in the R limma package. P values from moderated (paired) t-tests are adjusted for multiple hypotheses with the Benjamini-Hochberg procedure. Differentially expressed genes are determined using the criteria FCH >2, false discovery rate <0.05. Hierarchical clustering is performed with Euclidean distance and a McQuitty agglomeration scheme. Gene set enrichment analysis (GSEA) is performed to determine if the gene sets show statistically significant concordant differences between inductors. The overlap between genes in the murine transcriptome and their orthologs on the published human transcriptome is determined.

Western Blotting. Protein from primary murine keratinocytes is extracted using a protein lysis buffer (Abcam, Cambridge, MA, ab152163) and protease inhibitor cocktail (Sigma-Aldrich, P8340). The lysates are centrifuged at 12,000 rpm for 5 min at 4° C., and the supernatant is used in the following steps. An equal amount of protein is separated on SDS-polyacrylamide gels and transferred onto nitrocellulose membranes (Bio-Rad Laboratories, Hercules, CA, 162-0168). After blocking non-specific binding with 5% skim milk in TBST for 1 h at room temperature, the membranes are incubated for 16 h at 4° C. with primary antibodies against a-tubulin (Sigma-Aldrich), p38 (Cell Signaling Technology), and p-p38 (Cell Signaling Technology), followed by incubation with horseradish peroxidase-conjugated secondary antibodies for 1 h at room temperature. Signals are detected using ECL Prime Western Blotting Detection Reagent (GE Healthcare, Buckinghamshire, England, GE-RPN2024). Images are captured, and the density of the bands was measured using the ChemiDoc Touch Imaging System (Bio-Rad).

### Results

It is anticipated that daily topical treatment with BT032 will attenuate the development of anisomycin-induced psoriatic dermatitis in mice. It is also anticipated that increased ear thickness, scaly erythema, and histologic changes in mice induced by anisomycin are attenuated by BT032 treatment.

It is also anticipated that BT032 treatment will suppress IL-17A-induced expression of psoriasis-related genes, such as IL1A, IL1B, IL6, IL24, and CXCL1, in NHEKs. It is also anticipated that BT032 will suppress psoriasis-related gene expression levels in lesional skin from patients with psoriasis ex vivo. It is anticipated that BT032-treated specimens will exhibit lower expression levels of psoriasis-related inflammatory genes, such as IL1B, IL6, IL23A, and IL17A, than control specimens.

Accordingly, these results will show that the compounds of the present technology (e.g., BT032) are useful and effective in methods for the prevention and treatment of inflammasome-mediated dermatological diseases and conditions, such as psoriasis.

### Example 9: Probenecid Analogs of the Present Technology for the Prevention and Treatment of Inflammasome-Mediated Dermatological Disease—Recurrent Respiratory Papillomatosis

This example will demonstrate the capability of the probenecid analogs of the present technology (e.g., BT032) to treat or prevent recurrent respiratory papillomatosis in subjects.

### Background

Recurrent respiratory papillomatosis is a disease caused by the human papillomavirus that leads to growth of warts in the upper airway. Recent evidence suggests gain-of-function mutations in the NLRP1 gene can lead to inflammasome activation underlying the development of recurrent respiratory papillomatosis.

### Methods

Primary keratinocytes. Primary human keratinocytes are derived from skin biopsy specimens obtained from subjects with recurrent respiratory papillomatosis and healthy donors, obtained with informed consent. Primary keratinocyte cell lines will be will be grown and maintained.

Cell lines, cell culture, and transfections. HEK293T cells are purchased from American Type Culture Collection (CRL-3216) and cultured in DMEM and Glutamax (Invitrogen) plus 10% FCS. The patients' primary keratinocytes are derived from skin biopsy specimen. Control keratinocytes are single-donor adult human epidermal keratinocytes (e.g., Lonza; catalog no. 00192627, donors 34014, 30214, and 34015). Keratinocytes are maintained on mitomycin-C-inactivated 3T3-J2 feeder cells in Complete Green medium (DMEM/Ham's F-12 in a 2:1 ratio supplemented with 10% FBS, 180 nM adenine, 10 ng/mL EGF, 0.4 μg/mL hydrocortisone, 8.47 ng/mL cholera toxin, 5 μg/mL insulin, 1.36 ng/mL triiodothyronine, and 10 μMROCK inhibitor Y-27632). Immortalized N/TERT-1 keratinocytes are cultured in KSFMmedia (Life Technologies) supplemented with 300 μM CaCl₂). Transient transfection of HEK293T cells is performed using Lipofectamine 2000 (Life Technologies) at a 2:1 ratio according to the manufacturer's instructions. Transfection of immortalized keratinocytes is performed using FuGENE HD (Promega) at a 3:1 ratio according to the manufacturer's instructions. In the indicated experiments, keratinocytes are stimulated with 3 μM talabostat (MedChemExpress) for 16 h before analysis. Experimental groups for assessing the efficacy of BT032 in the treatment or prevention of recurrent respiratory papillomatosis are cultured with BT032 in a dose range of 1 μM to 350 μM for 2 hours prior to talabostat stimulation.

Western Blot Analysis. Cells are lysed in RIPA buffer with cOmplete protease inhibitor (Roche), and protein is quantified using the BCA protein assay (Pierce). For standard denaturing polyacrylamide gel electrophoresis (PAGE), lysate is subjected to reducing sodium dodecyl sulfate (SDS)-PAGE using Tris-glycine buffers and transferred to PVDF membranes (Immobilon), followed by detection with primary antibody against NLRP1 (R&D Systems; AF6788) and secondary anti-sheep HRP (R&D Systems; HAF016). Anti-GAPDH (Santa Cruz Biotechnology; FL-335) served as a loading control. Membranes are developed and detected using ECL Western blotting substrate (Pierce) and imaged on an Amersham Imager 600 (GE Healthcare). Blue natural PAGE (BN-PAGE) is performed with the Novex NativePAGE Bis-Tris gel system (Thermo Fisher Scientific).

Quantitative PCR. RNA is extracted from keratinocytes at indicated time points using TRIzol reagent (Invitrogen) according to the manufacturer's instructions. cDNA is synthesized with the SuperScript III First-Strand Synthesis System (Thermo Fisher Scientific) with random hexamers according to the manufacturer's instructions. Quantitative PCR (qPCR) is performed using TaqMan Universal PCR Master mix with the following FAM-MGB conjugated TaqMan Gene Expression Assays (Thermo Fisher Scientific) for NLRP1 (Hs00248187_m1) duplexed with VIC-MGB RNase P TaqMan Assay (4403328) as an endogenous control. qPCR is run on an Applied Biosystems 7500 Fast Real-Time PCR system. Gene expression is quantified by the 2-ddCt method.

Cytokine measurements. Human IL-1β, ILR1Ra, IL-6, TNF-α, and IL-18 cytokine levels are determined in serum of recurrent respiratory papillomatosis subjects and healthy controls by a magnetic bead-based multiplex assay using Luminex technology (Bio-Rad). GraphPad Prism 6.0 software is used for data analysis. ELISA for IL-1β (BD Biosciences) and IL-18 (R&D Systems) is performed on tissue culture supernatants according to the manufacturer's instructions.

PBMC Purification and Stimulation with TLR Ligands. PBMCs are isolated using Leucosep tubes (Greiner Bio-One) containing Ficoll density gradient medium. Cells are stored in RPMI-1640 medium supplemented with GlutaMAX (Gibco; 61870044) enriched with 10% FCS (Sigma-Aldrich; F7524) containing 10% DMSO (Sigma-Aldrich; D2650) at −150° C. until further use. PBMCs are thawed in 37° C. preheated complete medium (RPMI-1640 medium supplemented with GlutaMAX, 10% FCS, and 1% penicillin-streptomycin [10,000 U/mL; Gibco; 15140122], 1 mM sodium pyruvate [Gibco; 11360070], 1% nonessential amino acids [Gibco; 11140035], and 50 M 2-mercaptoethanol [Gibco; 31350010]). In the setting of functional testing, cells are left to recuperate for 30 min at 37° C. and 5% CO2 after removal of DMSO. PBMCs are stimulated for 24 h with TLR ligands, including HKLM (10e8/mL and 10e7/mL) and LPS (100 ng/mL and 10 ng/mL) (InvivoGen).

### Results

It is expected that treatment of keratinocytes from recurrent respiratory papillomatosis subjects will exhibit reduced release of IL-1β in the supernatants compared to baseline levels, demonstrating attenuation of inflammasome activation.

Accordingly, these results will show that the compounds of the present technology (e.g., BT032) are useful and effective in methods for the prevention and treatment of inflammasome-mediated dermatological diseases and conditions, such as recurrent respiratory papillomatosis.

### Example 10: Probenecid Analogs of the Present Technology for the Prevention and Treatment of Inflammasome-Mediated Dermatological Disease—Cryopyrin-Associated Periodic Syndromes (CAPS)

This example will assess the efficacy of the probenecid analogs of the present technology (e.g., BT032) on ex vivo samples from cryopyrin-associated periodic syndromes (CAPS) subjects.

### Background

Cyropyrin-associated period syndromes (CAPs) are a rare family of heterogeneous autoinflammatory diseases characterized by IL-1β-mediated systemic inflammation and clinical symptoms involving skin. CAPS is associated with an activating mutation in NLRP3. CAPS is categorized into three clinical subgroups of increasing severity: familial cold autoinflammatory syndrome (FCAS), Muckle-Wells syndrome (MWS), and neonatal-onset multisystem inflammatory disease (NOMID). Other autoinflammatory diseases with skin manifestations that feature elevated IL-1β include Schnitzler syndrome.

### Methods

Study population. CAPS patients with confirmed NLRP3 mutations and symptoms consistent with FCAS, MWS, or NOMID are included. Patients with clinical diagnoses of Schnitzler syndrome may also be included in the study. Voluntary blood samples are collected. Healthy blood donations are obtained by means of voluntary donation.

Collection of PBMC and serum. 45 mL whole blood is collected from patients into heparin tubes. Following donation, blood samples are maintained at room temperature and processed for PBMC isolation within 90 minutes of blood donation. Whole blood is diluted 1:1 in PBS and PBMCs are isolated using density gradient. Serum is removed from the top of the density gradient and stored at −80° C. PBMCs are removed and washed twice in PBS and counted using a haemocytometer. Isolated PBMC are plated at 2×10⁶ cell/mL in a 12 well plate in RPMI supplemented with 10% penicillin/streptomycin. Cells are left to rest for 2 hours at 37° C. 5% CO₂ before experimental treatment commences.

Inflammasome assay. PBMC are either left untreated or stimulated with 1 mg/ML LPS and incubated for one hour at 37° C. 5% CO₂. The media is then replaced with RPMI supplemented with 10% penicillin/streptomycin and BT032 is added in concentrations ranging from 5 nM to 500 nM. After 3 hours the supernatants are harvested, cells are lysed in SDS Laemmli sample buffer (125 mM Tris pH 6.8, 10% glycerol, 0.02% SDS, bromophenol blue). In cases where nigericin is used as an additional signal, PBMC is either left untreated or stimulated with 1 mg/IL LPS and incubated for three hours at 37° C. 5% CO₂. The media is then replaced with 10% penicillin/streptomycin and BT032 is added in concentrations ranging from 5 nM to 500 nM for one hour prior to the addition of 20 μM nigericin for 20 minutes. The supernatants are then harvested and cells are lysed.

ELISA. Human IL-1β ELISA kits are used to quantify IL-1β in cell supernatants and serum. Human TNF alpha and IL-6 are quantified by ELISA in cell supernatants. Free ASC are quantified by ELISA in serum samples. These kits utilize a microplate reader set at 450 nm to determine the optical density of the wells. Human AlphaLISA Detection Kits are used to quantify IL-1β in whole blood when read on an AlphaLISA-enabled spectrophotomenter at 615 nm.

Western Blot. Cells are lysed in SDS sample loading buffer and boiled for 5 minutes. Sample lysates are run on a SDS page gel, transferred to Immobilon-P PVDF membrane and probed for pro-IL-1β, IL-1β p17, IL-18, Vaspase-1, and beta actin. All antibodies are used at a dilution of 1:1000 in 5% powdered milk for 2 hours at room temperature, with the exception of beta actin which is used under the same conditions but at a dilution of 1:5000. Peroxidase-conjugated AffiniPure goat anti-rabbit, bovine anti-goat, and goat anti-mouse IgG are used as secondary antibodies at a dilution of 1:2000 in 5% powdered milk, prior to development with Immobilon Western Chemiluminescent HRP Substrate.

Expression of human NLRP3 mutants in NLRP3-deficient mouse macrophages. NLRP3-deficient iBMDM expressing Tet3G transactivator are transduced with amphotrophic tetrovirus encoding specific human NLRP3. To produce retrovirus, 1.5-2×10⁶ cells of packaging cell line per well are plated in a 6 well plate and left overnight at 37° C. 5% CO₂. The next day, cells are transfected with 4 μg of the plasmid DNA using 10 μL of Lipofectamine 2000 (Invitrogen). The next day, the medium is exchanged. 4×10⁵ recipient cells are seeded per well of a 6 well plate. The following day, the media of recipient NLRP3-deficient iBMDM is changed to DMEM+10% FBS+polybrene (2 μL/mL, Sigma). The retroviral supernatant from packaging cells is filtered through sterile 0.45 μm syringe filter. The retroviral supernatant is added dropwise to the recipient NLRP3 deficient iBMDM. After 24 hours, the medium is removed and the cells transferred to a 9 cm Petri dish. 1.5 mg/mL G418 and 6 μg/mL puromycin is added to the culture for selection. The cells are maintained in this matter until use in an inflammasome assay.

### Results

It is anticipated that ASC and mature IL-1β levels, which are higher in ex vivo PBMCs from CAPS patients than healthy donors, will be reduced in PBMCs from CAPS patients when treated with BT032. It is expected that BT032 will inhibit the production of IL-1β from CAPS patient PBMCs in response to LPS and impair IL-1β production in CAPS patients. It is further anticipated that BT032 will also reduce production of IL-1β in PBMCs from CAPS patients.

Accordingly, these results will show that the compounds of the present technology (e.g., BT032) are useful and effective in methods for the prevention and treatment of inflammasome-mediated dermatological diseases and conditions, such as dermatological manifestations of CAPS.

### Example 11: Probenecid Analogs of the Present Technology for the Prevention and Treatment of Inflammasome-Mediated Dermatological Disease—Dermal Inflammatory Challenge

This example will assess the efficacy of the probenecid analogs of the present technology (e.g., BT032) on inflammasome activation in healthy volunteers exposed to UVB irradiation.

### Objectives

| Objectives | Endpoints |
|---|---|
| To evaluate the | Dermal imaging |
| pharmacodynamic (PD) | Perfusion (laser speckle contrast imaging |
| properties of BT032 in | Erythema (multispectral imaging) |
| healthy volunteers after | Ex vivo inflammasome challenge |
| single ascending dose based | Cytokines (e.g., IL-1β, IL-18) |
| on dermal imaging and ex | Blister exudate analysis |
| vivo inflammasome | Cytokines (MSD) |
| challenges | Inflammasome proteins (MSD) |

### Design

This is a single-center, inflammatory challenge study in healthy volunteers, to evaluate inflammasome activation by UVB in healthy volunteers. Healthy volunteers will undergo a topical UVB challenge after intradermal dosing with BT032.

UV-B Challenge+BT032 (n=9; 3 Placebo, 6 Active):
Screening: 1 hour admission
Study day (day −1 to 1): 24 hours admission (baseline assessments, challenge, and 2, 4 hr blister, skin assessments)
Follow-up visit 1 (day 2): 1 hour admission (24 hr blister)
End of study visit (+7 days): 1 hour admission

Note: maximally 5 invasive assessments per volunteer.

Investigational drug (BT032)+UVB challenge. As part of the screening assessments, the subject's Fitzpatrick skin photo type is determined (type I-VI). The subject is first exposed to 6 different doses of UV-B, to determine the Minimal Erythemic Dose (MED) expressed in J/cm², using the six different slots of the UV-B lamp. Twenty-four hours (±2 hours) after the exposure of the 6 doses, the erythemic response of the skin to UV-B is assessed by two observers. The MED is determined visually, by observing which dose produces the first clearly discernible erythema. On the treatment days, the subject's skin is exposed to two minimal erythema doses (2MED) of UVB.

Subjects Groups. A total of 9 healthy volunteers will be enrolled. These subjects will undergo an UVB challenge after dosing with BT032.

### Main Inclusion Criteria:

1. Healthy subjects, 18 to 55 years of age, inclusive. Healthy status is defined by absence of evidence of any active or chronic disease following a detailed medical and surgical history, a complete physical examination including vital signs, 12-lead ECG, hematology, coagulation, blood chemistry, blood serology and urinalysis. In the case of uncertain or questionable results, tests performed during screening may be repeated before randomization to confirm eligibility or judged to be clinically irrelevant for healthy subjects;
2. Body mass index (BMI) between 18 and 30 kg/m² and a minimum weight of 50 kg, inclusive;
3. Fitzpatrick skin type I-III (Caucasian);
4. Subjects and their partners of childbearing potential must use effective contraception for the duration of the study;
5. Able and willing to give written informed consent and to comply with the study restrictions.

Main exclusion criteria. Eligible subjects must meet none of the following exclusion criteria at screening:
1. History of pathological scar formation (keloid, hypertrophic scar) or keloids or surgical scars in the target treatment area that in the opinion of the investigator, would limit or interfere with dosing and/or measurement in the trial;
2. History of skin cancer (basal cell carcinoma, squamous cell carcinoma, melanoma);
3. Have any current and/or recurrent clinically significant skin condition at the treatment area (i.e. atopic dermatitis); including tattoos;
4. History or presence of post-inflammatory hyperpigmentation.
5. Using immunosuppressive or immunomodulatory medication within 30 days prior to enrolment or planned to use during the course of the study;
6. Use of topical medication (prescription or over-the-counter [OTC]) within 30 days of study drug administration, or less than 5 half-lives (whichever is longer) in local treatment area;
7. Participation in an investigational drug or device study within 3 months prior to screening or more than 4 times a year;
8. Loss or donation of blood over 500 mL within three months prior to screening or donation of plasma within 14 days of screening;
9. Any (medical) condition that would, in the opinion of the investigator, potentially compromise the safety or compliance of the patient or may preclude the patient's successful completion of the clinical trial;
10. Chronic infection with HIV, hepatitis B (HBV) or hepatitis C (HCV). A positive HBV surface antigen (HBsAg) test at screening excludes a subject;
11. A history of ongoing, chronic or recurrent infectious disease;
12. Current smoker and/or regular user of other nicotine-containing products (e.g., patches);
13. History of or current drug or substance abuse considered significant by the PI (or medically qualified designee), including a positive urine drug screen.
14. Tanning due to sunbathing, excessive sun exposure or a tanning booth within 3 weeks of enrollment.
15. A minimal erythema dose (MED) higher than 355 mJ/cm² at screening. Applicable for the participants in the UVB-MITT population only.

Concomitant medications. No prescription medications, OTC medications, vitamin, herbal and dietary supplements will be permitted within 7 days prior to study drug administrations, or less than 5 half-lives (whichever is longer), and during the course of the study. Exception is paracetamol (up to 4 g/day) in case of local pain. Other medication may be allowed by the discretion of the investigator.

### Pharmacodynamic Endpoints:

Inflammasome-driven cytokines in whole blood challenge assay:
   IL-1β
   IL-18
   IL-6
   TNF
Inflammasome proteins such as NLRP3, ASC, NLRP1

### Results

It is anticipated that subjects treated with BT032 will exhibit a reduction in the activation of inflammasomes and reduced levels of inflammasome-driven cytokines (e.g., IL-1β, IL-18, IL-6, and/or TNF), and/or inflammasome proteins, such as NLRP3, ASC, and NLRP1, as compared to non-treated controls.

Accordingly, these results will show that the compounds of the present technology (e.g., BT032) are useful and effective in methods for the prevention and treatment of inflammasome-mediated dermatological diseases and conditions, such as those resulting from exposure to UVB irradiation.

### Example 12: BT032 Inhibits NLRP1-Induced Inflammasome Activation

This example demonstrates the efficacy of the probenecid analogs of the present technology (e.g., BT032) on inflammasome activation.

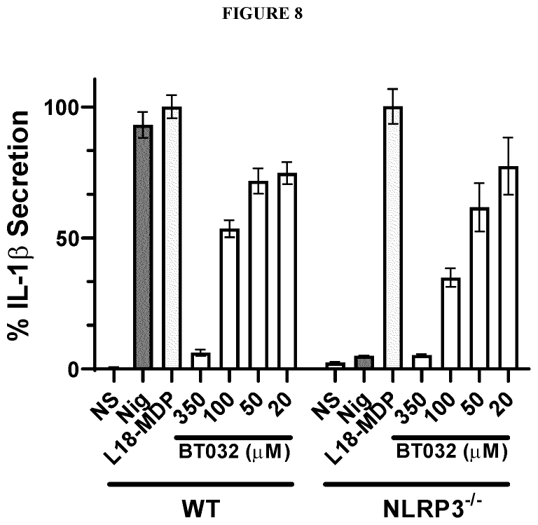

In vitro stimulation of murine macrophages. Immortalized BMDMs (wild type (WT) or NLRP3 deleted (NLRP3^{-/-})) grown in DMEM/10% FCS, 2 mM glutamine at 5% CO₂ were seeded at 4×10⁴ cells in 96 well format, 20 h prior to pre-treatment with LPS (100 ng/ml) for 3 h. Macrophages were treated with BT032 (20, 50, 100, 350 μM) or vehicle (DMSO) where indicated in serum-free media for 60 mins prior to challenge with NLRP1 agonist, L18-MDP (100 μg/mL; 16 hours); or the NLRP3 agonist nigericin (3 μM; 90 mins) where indicated, (NS=non-stimulated cells). Cultured supernatants were assayed for secreted IL-1β by ELISA according to manufacturer's instructions and are represented in FIG. 8 as the % maximal activation of untreated macrophages (“L18-MDP”). Pooled results of 3 independent experiments were conducted in triplicate (i.e., n=9) where activity was normalized as percentage of activity as related to the DMSO-treated control cells (“L18-MDP”) and non-stimulated (NS) cells.

Results. As shown by FIG. 8, treatment of either WT or NLRP3^{-/-} macrophages with BT032 dose-dependently inhibited NLRP1 (L18-MDP)-induced inflammasome activation as measured by IL-1β secretion. These results demonstrate that probenecid analog BT032 of the present technology is an NLRP1 inhibitor.

Accordingly, these results demonstrate that probenecid analog BT032 of the present technology, is useful in methods for inhibiting NLRP1 inflammasome activation and for treating or preventing inflammasome-mediated dermatological diseases or conditions.

### Example 13: BT032 Directly Binds to NLRP1 and NLRP3

This example demonstrates the mechanism by which BT032 (also referred to herein as “ADS032”) inhibited NLRP1 and NLRP3 function.

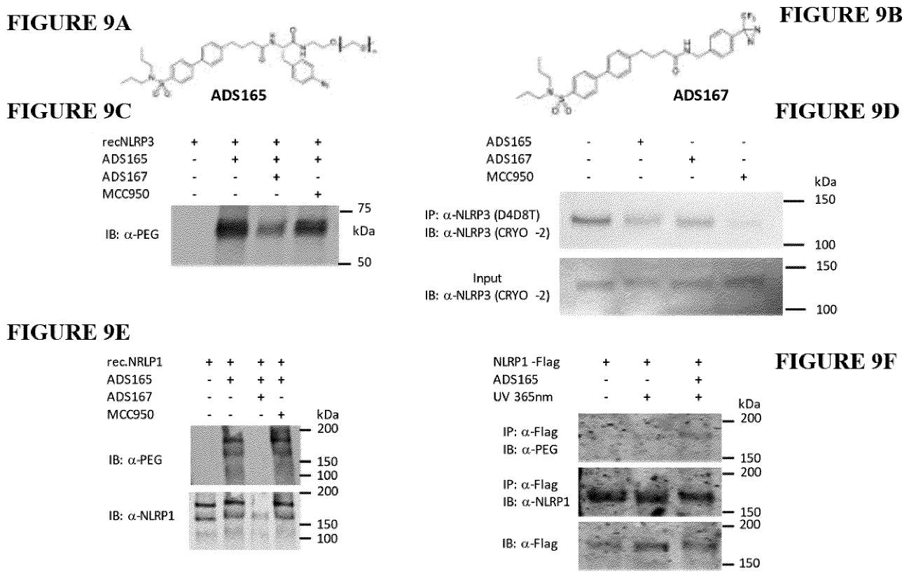

To verify interaction with target proteins, a photo-affinity labeling strategy (MacKinnon+Taunton, 2009) was developed where two modified versions of ADSO32 (1) containing an alkyne photoactivatable moiety linked with a PEG550 moiety to allow visualization (FIG. 9A) termed ADS165; and (2) ADSO32 linked with a diazirine photo-affinity label, (FIG. 9B) termed ADS167 were synthesized. Both photo-affinity labels covalently cross-link to molecular targets when exposed to ultraviolet light (365 nm). Initially, it was confirmed that both ADS165 and ADS167 were still able to inhibit NLRP3-induced IL-1β secretion from iBMDMs, (although with reduced efficacy as compared to ADS032), suggesting commensurate target engagement (data not shown). As can be seen in FIG. 9C, recombinant NLRP3 minus the leucine rich repeat region (molecular weight approximately 66 kDa), co-incubated with ADS165 (ADSO32 linked to PEG) and exposed to UV light can be identified by immunoblot with a-PEG antibody, suggesting that ADS165 is covalently linked to NLRP3. Importantly, pre-incubation of NLRP3 with the non-PEG tagged ADS032 moiety, ADS167 and exposure to UV light, inhibited the ability of ADS165 to label NLPR3, suggesting that both photo-affinity labels are competing for the same binding site on NLRP3. Conversely, MCC950, which has previously been demonstrated to bind the Walker B domain of NLRP3, did not inhibit ADS165 binding with NLPR3, although as MCC950 does not covalently bind NLRP3, it may not have been able to covalently compete with NLRP3 for binding efficacy.

It was then determined whether the ADSO32 binding site may be within the NACHT domain of NLRP3. As previously described, the D4D8T NLRP3 antibody recognizes residues around Alanine 306 which is proximal to the Walker B motif within the NACHT domain of NLRP3. NLRP3 was immunoprecipitated with this antibody from iBMDMs and it was found that pre-treatment and photo-labelling macrophages with either ADS165 or ADS167, reduced the efficacy of D4D8T-mediated NLRP3 immunoprecipitation. Consistent with previous reports, MCC950 also blocked NLPR3 precipitation.

Next, it was determined whether ADSO32 could interact with NLRP1. Consistent with observations for NLRP3, it was found that while ADS165 was able to ‘tag’ recombinant full length NLRP1 with PEG (FIG. 9E, second column), ADS167 could ablate interaction, while MCC950 had no effect, suggesting that ADS32 directly binds the NACHT domain of NLRP1, analogous to that observed for NLRP3. To further investigate this interaction, ectopically expressed Flag-tagged NLRP1 was immunoprecipitated from cell lysates pretreated or not with ADS165 and exposed to UV light. As can be seen in FIG. 9F, only PEG-tagged NLRP1-Flag in cells exposed to both ADS165 and UV could be observed, consistent with ADS032 directly interacting with NLRP1 in vitro.

Overall, these results suggest that ADS032 (or “BT032”) directly interacts proximal to the Walker B motif within the NACHT domain of both NLRP1 and NLRP3, thus inhibiting inflammasome activation and formation of the inflammasome complex.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

What is claimed is:

1. A compound having the structure of Formula I, 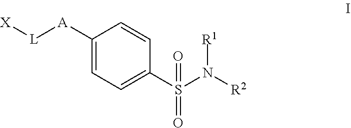
tautomers thereof and/or pharmaceutically acceptable salts thereof;
wherein
A is absent, or is selected from the group consisting of C(O)N(R³), phenylene, oxazolylene, thiazolylene, piperidinylene, and 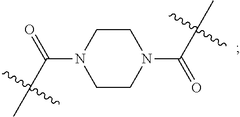 [SMILES: *C(=O)N1CCN(C(*)=O)CC1]
L is absent or C₁₋₁₀ alkylene;
X is H, CHO, COOH, C(O)NR⁴R⁵, COOR⁶, NH₂ or NHR;
R is 2-chloropyrimidin-4-yl;
R¹ and R² are independently a substituted or unsubstituted C₁₋₆ alkyl group, or one of R¹ and R² is H, and the other is cyclohexyl-NH—C(O), or R¹ and R² together are a C₄₋₆ alkylene group and form a 5-, 6-, or 7-member ring with the nitrogen to which they are attached, said ring optionally substituted with a phenyl group;
R³ and R⁴ are independently selected from H or a C₁₋₆ alkyl group;
R⁵ is selected from H, PEG, or a C₁₋₆ alkyl group; and
R⁶ is selected from a substituted or unsubstituted C₁₋₁₀ alkyl, C₂₋₁₀ alkenyl, or C₇₋₁₄ aralkyl group.

2. The compound of claim 1, wherein A is absent.

3. The compound of claim 1, wherein A is C(O)N(R³).

4. The compound of claim 3, wherein R³ is H or methyl.

5. The compound of claim 1, wherein A is phenylene, oxazolylene, thiazolylene, piperidinylene or

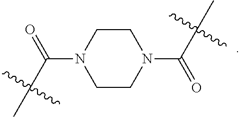 [SMILES: *C(=O)N1CCN(C(*)=O)CC1]

6. The compound of any one of claims 1-5, wherein A is phenylene, oxazolylene, thiazolylene, or piperidinylene.

7. The compound of any one of claims 1-6, wherein L is absent.

8. The compound of any one of claims 1-6, wherein L is a C₁₋₁₀ alkylene.

9. The compound of any one of claims 1-8 wherein X is H, CHO, COOH, C(O)NR⁴R⁵, NH₂ or NHR.

10. The compound of any one of claims 1-8, wherein X is H.

11. The compound of any one of claims 1-8, wherein X is COOH.

12. The compound of any one of claims 1-8, wherein X is COOR⁶.

13. The compound of any one of claims 1-8, wherein X is C(O)NR⁴R⁵.

14. The compound of any one of claims 1-8, wherein X is NH₂ or NHR.

15. The compound of any one of claims 1-14, wherein each of R¹ and R² is independently a C₁₋₆ alkyl, optionally substituted with one or more F, OH, CF₃, C₃₋₇ cycloalkyl group or SO₂-alkyl.

16. The compound of any one of claims 1-14, wherein R¹ and R² together are a C₄₋₆ alkylene group and form a 5-, 6-, or 7-member ring with the nitrogen to which they are attached.

17. The compound of any one of claims 1-14, wherein one of R¹ and R² is H, and the other is cyclohexyl-NH—C(O).

18. The compound of claim 1, wherein
A is absent, or is selected from the group consisting of C(O)N(R³), phenylene, oxazolylene, thiazolylene, and piperidinylene,
L is absent or C₁₋₁₀ alkylene;
X is H, COOH, or NH₂;
R¹ and R² are independently a substituted or unsubstituted C₁₋₆ alkyl group; and
R³ is selected from H or a C₁₋₆ alkyl group.

19. The compound of claim 18, wherein A is absent and L is C₃₋₁₀ alkylene.

20. The compound of claim 18, wherein A is phenylene, oxazolylene, thiazolylene, or piperidinylene, and L is absent or a C₁₋₅ alkylene.

21. The compound of claim 1, or a pharmaceutically acceptable salt thereof, wherein the compound is selected from the group consisting of:

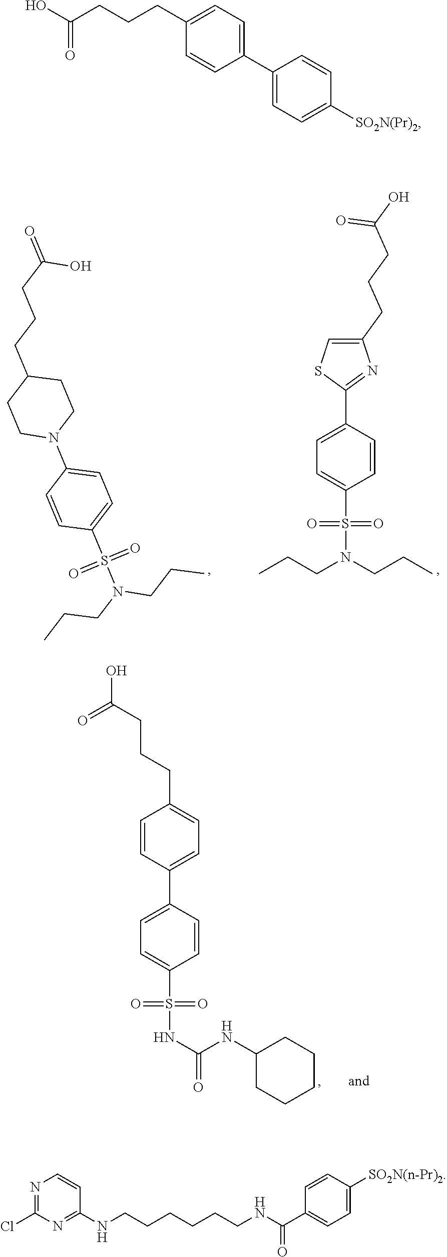

22. A pharmaceutical composition comprising a compound, tautomer thereof, and/or pharmaceutically acceptable salt of any one of claims 1-21 and a pharmaceutically acceptable carrier.

23. A method for treating or preventing an inflammasome-mediated dermatological disease or condition in a mammalian subject in need thereof, comprising administering to the subject a therapeutically effective amount of a compound, tautomer thereof, and/or pharmaceutically acceptable salt of any one of claims 1-21 or the pharmaceutical composition of claim 22.

24. The method of claim 23, wherein the inflammasome-mediated dermatological disease or condition is associated with NLRP1 inflammasome activation and/or NLRP3 inflammasome activation.

25. The method of claim 23 or claim 24, wherein the compound, tauotomer thereof, and/or pharmaceutically acceptable salt or the pharmaceutical composition comprises:

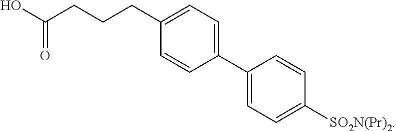

26. The method of claim 23 or claim 24, wherein the compound, tauotomer thereof, and/or pharmaceutically acceptable salt or the pharmaceutical composition comprises:

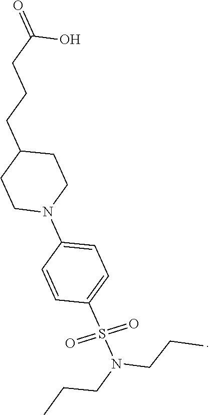 [SMILES: CCCN(CCC)S(=O)(=O)c1ccc(N2CCC(CCCC(=O)O)CC2)cc1]

27. The method of claim 23 or claim 24, wherein the compound, tauotomer thereof, and/or pharmaceutically acceptable salt or the pharmaceutical composition comprises:

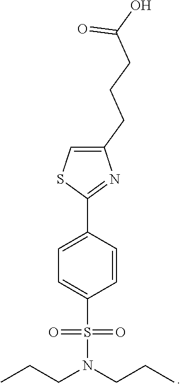 [SMILES: CCCN(CCC)S(=O)(=O)c1ccc(-c2nc(CCCC(=O)O)cs2)cc1]

28. The method of claim 23 or claim 24, wherein the compound, tauotomer thereof, and/or pharmaceutically acceptable salt or the pharmaceutical composition comprises:

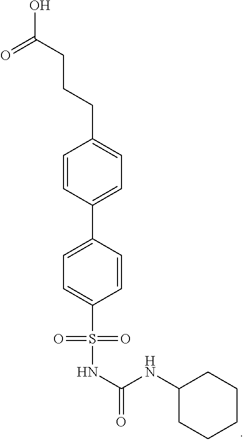 [SMILES: O=C(O)CCCc1ccc(-c2ccc(S(=O)(=O)NC(=O)NC3CCCCC3)cc2)cc1]

29. The method of claim 23 or claim 24, wherein the compound, tauotomer thereof, and/or pharmaceutically acceptable salt or the pharmaceutical composition comprises:

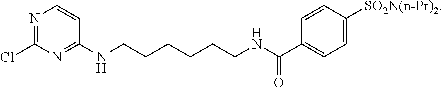

30. The method of any one of claims 25-29, wherein the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

31. The method of any one of claims 23-30, wherein the administering step is selected from the group consisting of topical administration, intradermal administration, intranasal administration, intramuscular administration, subcutaneous administration, administration by inhalation, and oral administration.

32. The method of any one of claims 23-31, wherein the treating or preventing inflammasome-mediated dermatological diseases or conditions comprises reducing the level of one or more inflammatory cytokines in the subject as compared to an untreated control subject.

33. The method of claim 32, wherein the one or more inflammatory cytokines is selected from the group consisting of MCP-1, IL-18, IL-1β, IL-6, TNF-α, and any combination thereof.

34. The method of claim 33, wherein the one or more inflammatory cytokines is IL-1β.

35. The method of any one of claims 25-34, wherein the treating or preventing inflammasome-mediated dermatological disease or condition comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject.

36. The method of any one of claims 23-35, wherein the treating or preventing inflammasome-mediated dermatological disease or condition comprises reducing apoptosis-associated speck-like protein containing a caspase activating and recruitment domain (ASC) speck formation in the subject as compared to an untreated control subject.

37. Use of a composition in the preparation of a medicament for treating or preventing an inflammasome-mediated dermatological disease or condition in a subject in need thereof, wherein the composition comprises a therapeutically effective amount of a compound, tautomer thereof, and/or pharmaceutically acceptable salt of any one of claims 1-21 or the pharmaceutical composition of claim 22.

38. The use of claim 37, wherein the inflammasome-mediated dermatological disease or condition is associated with NLRP1 inflammasome activation and/or NLRP3 inflammasome activation.

39. The use of claim 37 or claim 38, wherein the compound, tauotomer thereof, and/or pharmaceutically acceptable salt or the pharmaceutical composition comprises:

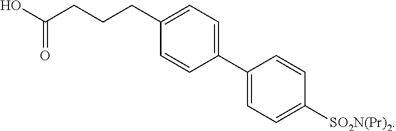

40. The use of claim 37 or claim 38, wherein the compound, tauotomer thereof, and/or pharmaceutically acceptable salt or the pharmaceutical composition comprises:

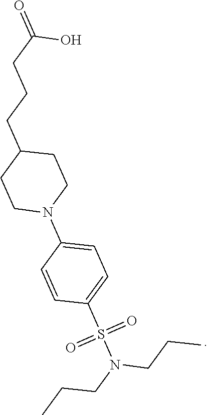 [SMILES: CCCN(CCC)S(=O)(=O)c1ccc(N2CCC(CCCC(=O)O)CC2)cc1]

41. The use of claim 37 or claim 38, wherein the compound, tauotomer thereof, and/or pharmaceutically acceptable salt or the pharmaceutical composition comprises:

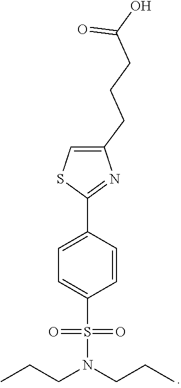 [SMILES: CCCN(CCC)S(=O)(=O)c1ccc(-c2nc(CCCC(=O)O)cs2)cc1]

42. The use of claim 37 or claim 38, wherein the compound, tauotomer thereof, and/or pharmaceutically acceptable salt or the pharmaceutical composition comprises:

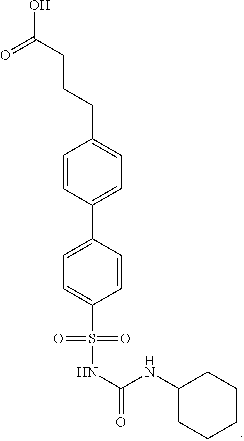 [SMILES: O=C(O)CCCc1ccc(-c2ccc(S(=O)(=O)NC(=O)NC3CCCCC3)cc2)cc1]

43. The use of claim 37 or claim 38, wherein the compound, tauotomer thereof, and/or pharmaceutically acceptable salt or the pharmaceutical composition comprises:

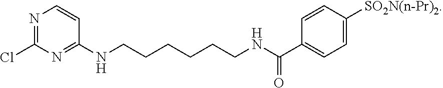

44. The use of any one of claims 37-43, wherein the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

45. The use of any one of claims 37-44, wherein the administering step is selected from the group consisting of topical administration, intradermal administration, intranasal administration, intramuscular administration, subcutaneous administration, administration by inhalation, and oral administration.

46. The use of any one of claims 37-45, wherein the treating or preventing inflammasome-mediated dermatological disease or condition comprises reducing the level of one or more inflammatory cytokines in the subject as compared to an untreated control subject.

47. The use of claim 46, wherein the one or more inflammatory cytokines is selected from the group consisting of MCP-1, IL-18, IL-1β, IL-6, TNF-α, and any combination thereof.

48. The use of claim 47, wherein the one or more inflammatory cytokines is IL-1β.

49. The use of any one of claims 37-48, wherein the treating or preventing dermatological disease or condition comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject.

50. The use of any one of claims 37-49, wherein the treating or preventing inflammasome-mediated dermatological disease or condition comprises reducing apoptosis-associated speck-like protein containing a caspase activating and recruitment domain (ASC) speck formation in the subject as compared to an untreated control subject.

51. A compound, tautomer thereof, and/or pharmaceutically acceptable salt of any one of claims 1-21 or the pharmaceutical composition of claim 22, for use in treating or preventing an inflammasome-mediated dermatological disease or condition in a subject in need thereof.

52. The compound, tautomer, pharmaceutically acceptable salt, or pharmaceutical composition of claim 51, for use wherein the inflammasome-mediated dermatological disease or condition is associated with NLRP1 inflammasome activation and/or NLRP3 inflammasome activation.

53. The compound, tautomer, pharmaceutically acceptable salt, or pharmaceutical composition of claim 51 or claim 52, for use wherein the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

54. The compound, tautomer, pharmaceutically acceptable salt, or pharmaceutical composition of any one of claims 51-53, for use wherein the compound, tauotomer thereof, and/or pharmaceutically acceptable salt or the pharmaceutical composition comprises:

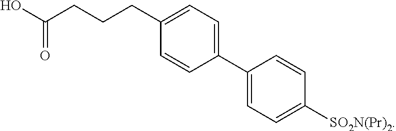

55. The compound, tautomer, pharmaceutically acceptable salt, or pharmaceutical composition of any one of claims 51-53, for use wherein the compound, tauotomer thereof, and/or pharmaceutically acceptable salt or the pharmaceutical composition comprises:

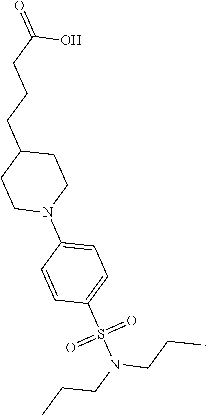 [SMILES: CCCN(CCC)S(=O)(=O)c1ccc(N2CCC(CCCC(=O)O)CC2)cc1]

56. The compound, tautomer, pharmaceutically acceptable salt, or pharmaceutical composition of any one of claims 51-53, for use wherein the compound, tauotomer thereof, and/or pharmaceutically acceptable salt or the pharmaceutical composition comprises:

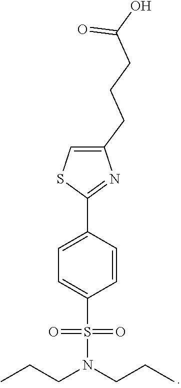 [SMILES: CCCN(CCC)S(=O)(=O)c1ccc(-c2nc(CCCC(=O)O)cs2)cc1]

57. The compound, tautomer, pharmaceutically acceptable salt, or pharmaceutical composition of any one of claims 51-53, for use wherein the compound, tauotomer thereof, and/or pharmaceutically acceptable salt or the pharmaceutical composition comprises:

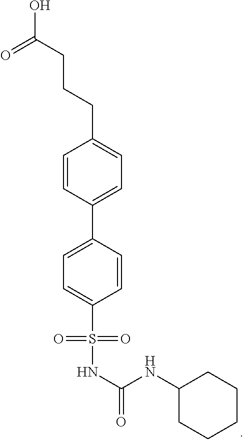 [SMILES: O=C(O)CCCc1ccc(-c2ccc(S(=O)(=O)NC(=O)NC3CCCCC3)cc2)cc1]

58. The compound, tautomer, pharmaceutically acceptable salt, or pharmaceutical composition of any one of claims 51-53, for use wherein the compound, tauotomer thereof, and/or pharmaceutically acceptable salt or the pharmaceutical composition comprises:

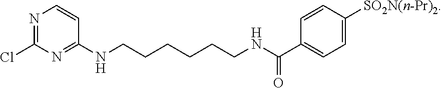

59. The compound, tautomer, pharmaceutically acceptable salt, or pharmaceutical composition of any one of claims 51-58, for use wherein the inflammasome-mediated dermatological disease or condition is selected from the group consisting of: skin-related genetic disorders such as NAIAD (NLRP1-associated auto-inflammation with arthritis and dyskeratosis), systemic sclerosis, multiple self-healing palmoplantar carcinoma (MSPC), and familial keratosis lichenoides chronica (FKLC); cryopyrin-associated periodic syndromes (CAPS) skin manifestations, Schnitzler syndrome, psoriasis, dermatitis, eczema, vitiligo, skin damage resulting from UV irradiation, and papillomas such as recurrent respiratory papillomatosis.

60. The compound, tautomer, pharmaceutically acceptable salt, or pharmaceutical composition of any one of claims 51-59, for use wherein the administering step is selected from the group consisting of topical administration, intradermal administration, intranasal administration, intramuscular administration, subcutaneous administration, administration by inhalation, and oral administration.

61. The compound, tautomer, pharmaceutically acceptable salt, or pharmaceutical composition of any one of claims 51-60, for use wherein the treating or preventing inflammasome-mediated dermatological disease or condition comprises reducing the level of one or more inflammatory cytokines in the subject as compared to an untreated control subject.

62. The compound, tautomer, pharmaceutically acceptable salt, or pharmaceutical composition of claim 61, for use wherein the one or more inflammatory cytokines is selected from the group consisting of MCP-1, IL-18, IL-1β, IL-6, TNF-α, and any combination thereof.

63. The compound, tautomer, pharmaceutically acceptable salt, or pharmaceutical composition of claim 62, for use wherein the one or more inflammatory cytokines is IL-1β.

64. The compound, tautomer, pharmaceutically acceptable salt, or pharmaceutical composition of any one of claims 51-63, for use wherein the treating or preventing inflammasome-mediated dermatological disease or condition comprises reducing one or more of: recurrent fever; widespread skin dyskeratosis; arthritis; elevated biologic markers of inflammation such as MCP-1, IL-18, IL-1β, IL-6, TNF-α, and/or elevated inflammasome proteins such as apoptosis-associated speck-like protein containing a caspase recruitment domain (ASC), NLRP3, or NLRP1; autoimmunity with a high transitional B-cell level; tightening of the skin; joint pain; exaggerated response to cold (Raynaud's disease); heartburn; urticaria; skin plaques; scaly erythema; rash; and macules as compared to an untreated control subject.

65. The compound, tautomer, pharmaceutically acceptable salt, or pharmaceutical composition of any one of claims 51-64, wherein the treating or preventing inflammasome-mediated dermatological disease or condition comprises reducing apoptosis-associated speck-like protein containing a caspase activating and recruitment domain (ASC) speck formation in the subject as compared to an untreated control subject.
